(19) United States
(12) Patent Application Publication
(10) Pub. No.: US 2023/0373974 A1
(43) Pub. Date: Nov. 23, 2023

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Thomas CLEVELAND, San Marcos, CA (US); Timothy A. DWIGHT, Quincy, MA (US); Bryan A. FRIEMAN, La Jolla, CA (US); Peter GROOTENHUIS, Del Mar, CA (US); Sara Sabina HADIDA RUAH, La Jolla, CA (US); Yoshihiro ISHIHARA, San Diego, CA (US); Mark Thomas MILLER, Rancho Santa Fe, CA (US); Alina SILINA, Needham, MA (US); Jinglan ZHOU, San Diego, CA (US)

(21) Appl. No.: 18/030,520

(22) PCT Filed: Oct. 6, 2021
(86) PCT No.: PCT/US2021/053865

§ 371 Date: Apr. 6, 2023

Related U.S. Application Data
(60) Provisional application No. 63/088,883, filed on Oct. 7, 2020.

(51) Int. Cl. C07D 403/12; C07D 239/70; C07D 279/08; C07D 241/44; C07D 471/04; C07D 277/82; C07D 513/04; C07D 277/84; C07D 239/84; C07D 487/04; C07D 231/56; C07D 215/38; C07D 217/22; C07D 491/048; C07D 235/30; A61K 45/06

(52) U.S. Cl. CPC C07D 403/12; C07D 239/70; C07D 279/08; C07D 241/44; C07D 471/04; C07D 277/82; C07D 513/04; C07D 277/84; C07D 239/84; C07D 487/04; C07D 231/56; C07D 215/38; C07D 217/22; C07D 491/048; C07D 235/30; A61K 45/06

(57) ABSTRACT

This disclosure provides modulators of Cystic Fibrosis Transmembrane Conductance Regulator (CFTR) having the structure: (I), pharmaceutical compositions, containing at least one such modulator, methods of treatment of cystic fibrosis using such modulators and pharmaceutical compositions, combination therapies, and processes and intermediates for making such modulators.

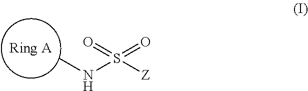

This application claims the benefit of priority of U.S. Provisional Application No. 63/088,883, filed Oct. 7, 2020, the contents of which are incorporated by reference herein in their entirety.

The disclosure relates to modulators of Cystic Fibrosis Transmembrane Conductance Regulator (CFTR), pharmaceutical compositions containing the modulators, methods of treatment of cystic fibrosis using such modulators and pharmaceutical compositions, combination therapies, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl- channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing.

Formula I encompasses compounds falling within the following structure:

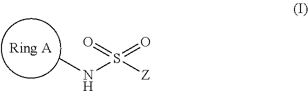

tautomers thereof, deuterated derivatives of those compounds or tautomers, or pharmaceutically acceptable salts of any of the foregoing, wherein: Ring A is a bicyclic or tricyclic ring system selected from:

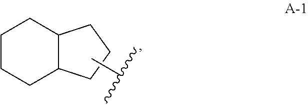

wherein Ring A-1 contains one or more unsaturated bonds and 2 to 3 ring carbon atoms that are independently replaced by nitrogen or sulfur;

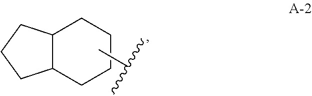

wherein Ring A-2 contains one or more unsaturated bonds and 1 to 3 ring carbon atoms that are replaced by nitrogen;

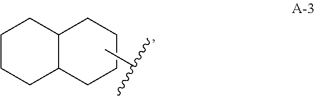

wherein Ring A-3 contains one or more unsaturated bonds and 1 to 3 ring carbon atoms that are independently replaced by nitrogen or sulfur;

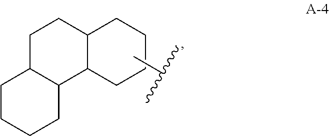

wherein Ring A-4 contains one or more unsaturated bonds and 1 to 3 ring carbon atoms that are replaced by nitrogen;

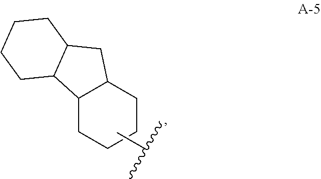

wherein Ring A-5 contains one or more unsaturated bonds and 1 to 3 ring carbon atoms that are independently replaced by nitrogen or oxygen;

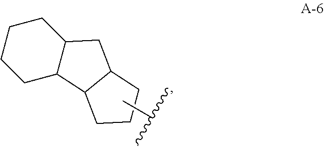

wherein Ring A-6 contains one or more unsaturated bonds and 2 to 3 ring carbon atoms that are independently replaced by nitrogen or sulfur; and

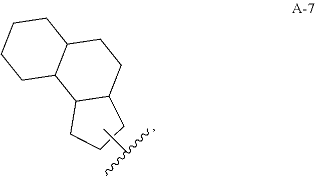

wherein Ring A-7 contains one or more unsaturated bonds and 2 to 3 ring carbon atoms that are independently replaced by nitrogen or sulfur;

Ring A is optionally substituted with 1 to 3 R¹ groups; wherein each R¹ is independently selected from:
halogen,
—C₁-C₆ alkyl optionally substituted with 1 to 3 groups selected from halogen, —OH, and —C₁-C₄ alkoxy;
phenyl optionally substituted with 1 to 3 groups selected from halogen, —CN, —C₁-C₆ alkyl (which is optionally further substituted with 1 to 3 groups selected from halogen and —OH), and —C₁-C₆ alkoxy;
—O-phenyl optionally substituted with 1 to 3 groups selected from halogen, —CN, and —C₁-C₆ alkyl (which is optionally further substituted with 1 to 3 groups selected from halogen and —OH); and
piperidinyl; and
Z is selected from:
phenyl optionally substituted with 1 or 2 groups independently selected from —NH₂ (optionally substituted with 1-2 groups selected from —C₁-C₃ alkyl), —C₁-C₃ alkyl, and —C₁-C₃ alkoxy;
pyrazolyl optionally substituted with 1 or 2 groups independently selected from halogen, —C₁-C₃ alkyl, and —C₁-C₃ alkoxy;
imidazolyl;
1,4-benzodioxanyl; and
pyridinyl optionally substituted with NH₂;
provided that the compound is not one of the following:
4-methyl-N-(1-methylisoquinolin-3-yl)benzenesulfonamide;
N-(4,5,6,7-tetrahydro-6-methyl-2-benzothiazolyl)benzenesulfonamide; and
N-[4,5,6,7-tetrahydro-1-(2-pyridinyl)-1H-indazol-4-yl]benzenesulfonamide.

Formula I also includes Compounds 1-61 and 63-95 (e.g., Compounds 1-7, 9-15, 20, 23, 25-40, 42-48, 50, 51, 53, 55, 56, 58-61, 70, 72, 73, 76, 76, 77, 79), tautomers of those compounds, deuterated derivatives of any of the compounds and tautomers, and pharmaceutically acceptable salts of any of the foregoing.

Another aspect of the disclosure provides pharmaceutical compositions comprising at least one compound chosen from the novel compounds disclosed herein, pharmaceutically acceptable salts thereof, and deuterated derivatives of any of the foregoing, and at least one pharmaceutically acceptable carrier, which compositions may further include at least one additional active pharmaceutical ingredient. Thus, another aspect of the disclosure provides methods of treating the CFTR-mediated disease cystic fibrosis comprising administering at least one of compound chosen from the novel compounds disclosed herein, pharmaceutically acceptable salts thereof, and deuterated derivatives of any of the foregoing, and at least one pharmaceutically acceptable carrier, optionally as part of a pharmaceutical composition comprising at least one additional component, to a subject in need thereof.

In certain embodiments, the pharmaceutical compositions of the disclosure comprise at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing. In some embodiments, compositions comprising at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing may optionally further comprise (a) at least one compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo [d][1,3]dioxol-5-yl)cyclopropanecarboxamido)-3-methylpyridin-2-yl)benzoic acid (lumacaftor), and deuterated derivatives and pharmaceutically acceptable salts of tezacaftor and lumacaftor; and/or (b) at least one (i.e., one or more) compound(s) chosen from N-(2,4-di-tert-butyl-5-hydroxyphenyl)-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Another aspect of the disclosure provides uses of the compounds and pharmaceutical compositions of the disclosure in methods of treating the CFTR-mediated disease cystic fibrosis that comprise administering to a patient in need thereof at least one compound chosen from the novel compounds disclosed herein, deuterated derivatives thereof, and pharmaceutically acceptable salts of any of the foregoing, and optionally further administering one or more additional CFTR modulating agents selected from tezacaftor, ivacaftor, deutivacaftor, lumacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

A further aspect of the disclosure provides intermediates and methods for making the compounds and compositions disclosed herein.

### Definitions

“Chosen from” and “selected from” are used interchangeably herein.

Compounds 1-95 in this disclosure is intended to represent a reference to each of Compounds 1 through 95 individually and a reference to groups of compounds, such as, e.g., Compound 1-95; Compounds 1-7, 9-15, 20, 23, 25-40, 42-48, 50, 51, 53, 55, 56, 58-61, 70, 72, 73, 76, 76, 77, 79.

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

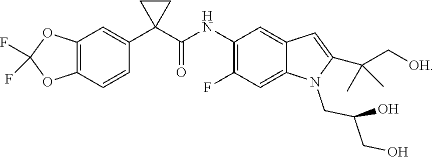 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated herein by reference.

“Ivacaftor” as used throughout this disclosure refers to N-(2,4-di-tert-butyl-5-hydroxyphenyl)-1,4-dihydro-4-oxoquinoline-3-carboxamide, which is depicted by the structure:

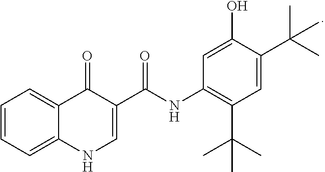 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

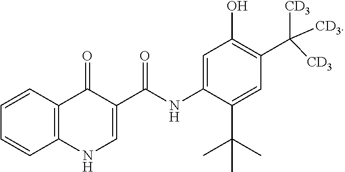 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a pharmaceutically acceptable salt or a further deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

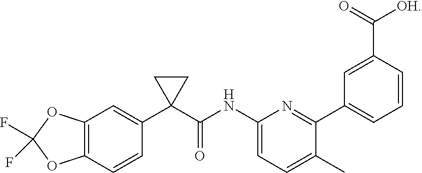 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using lumacaftor are disclosed in WO 2007/056341, WO 2009/073757, and WO 2009/076142, each of which is incorporated herein by reference.

As used herein, the term “alkyl” refers to a saturated or partially saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which one or more bonds between carbon atoms may be a double (alkenyl) or triple (alkynyl) bond. Alkyl groups may be substituted or unsubstituted.

As used herein, the term “haloalkyl group” refers to an alkyl group substituted with one or more halogen atoms, e.g., fluoroalkyl, which is an alkyl group substituted with one or more fluorine atoms.

The term “alkoxy” as used herein refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon group having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl” as used herein is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The term “heteroaryl ring” as used herein refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline.

As used herein, the term “heterocyclyl ring” refers to a non-aromatic hydrocarbon containing 3 to 12 atoms in a ring (such as, for example 3-10 atoms) comprising at least one ring atom that is a heteroatom, such as O, N, or S and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom. In chemical structures, deuterium is represented as “D.” In some embodiments, the one or more hydrogens replaced by deuterium are part of an alkyl group. In some embodiments, the one or more hydrogens replaced by deuterium are part of a methyl group.

The phrase “deuterated derivatives and pharmaceutically acceptable salts of [a specified compound or compounds],” as used herein, refers to deuterated derivatives of the compound or compounds as well as pharmaceutically acceptable salts of the compound or compounds and pharmaceutically acceptable salts of the deuterated derivative of the compound or compounds.

The phrase “and deuterated derivatives and pharmaceutically acceptable salts thereof” is used interchangeably with “and deuterated derivatives and pharmaceutically acceptable salts thereof of any of the forgoing” in reference to one or more compounds or formulae of the disclosure. These phrases are intended to encompass pharmaceutically acceptable salts of any one of the referenced compounds, deuterated derivatives of any one of the referenced compounds, and pharmaceutically acceptable salts of those deuterated derivatives.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

As used herein, “CFTR” means cystic fibrosis transmembrane conductance regulator.

As used herein, the term “CFTR modulator” refers to a compound that increases the activity of CFTR. The increase in activity resulting from a CFTR modulator includes but is not limited to compounds that correct, potentiate, stabilize and/or amplify CFTR.

As used herein, the term “CFTR corrector” refers to a compound that facilitates the processing and trafficking of CFTR to increase the amount of CFTR at the cell surface. The novel compounds disclosed herein are CFTR correctors.

As used herein, the term “CFTR potentiator” refers to a compound that increases the channel activity of CFTR protein located at the cell surface, resulting in enhanced ion transport. Ivacaftor and deutivacaftor disclosed herein are CFTR potentiators. It will be appreciated that when a description of a combination of compound selected from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and other specified CFTR modulating agents is provided herein, reference to “ivacaftor or deutivacaftor” in connection with the combination means that either ivacaftor or deutivacaftor, but not both, is included in the combination.

As used herein, the term “active pharmaceutical ingredient” or “therapeutic agent” (“API”) refers to a biologically active compound.

The terms “patient” and “subject” are used interchangeably and refer to an animal, including a human.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the value is measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/l).

As used herein, the term “room temperature” or “ambient temperature” means 15° C. to 30° C.

It will be appreciated that certain compounds of this disclosure may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

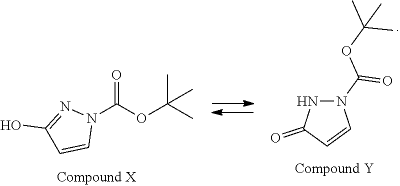

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### DETAILED DESCRIPTION OF EMBODIMENTS

In addition to compounds of Formula I, tautomers thereof, deuterated derivatives of those compounds and tautomers, and pharmaceutically acceptable salts of any of the foregoing, the disclosure provides compounds of Formula I, tautomers thereof, deuterated derivatives of those compounds and tautomers, and pharmaceutically acceptable salts of any of the foregoing.

For example, in some embodiments, the optionally substituted Ring A in the compound of Formula I is selected from

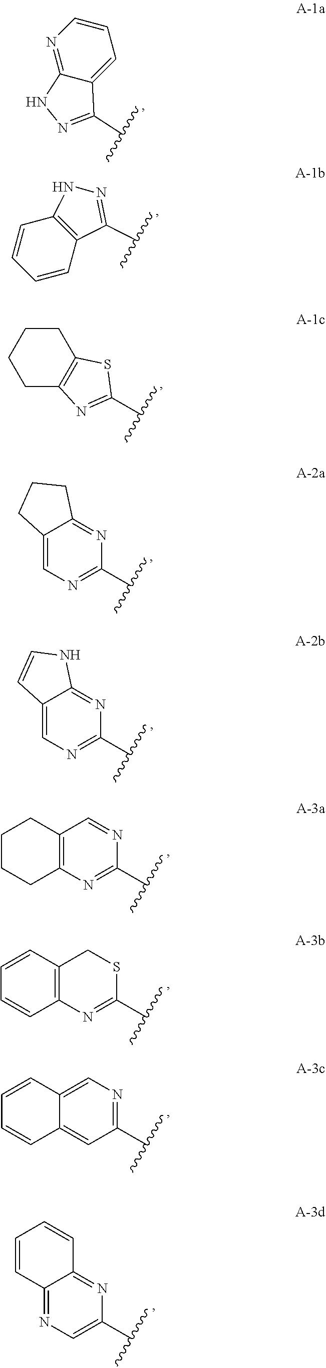

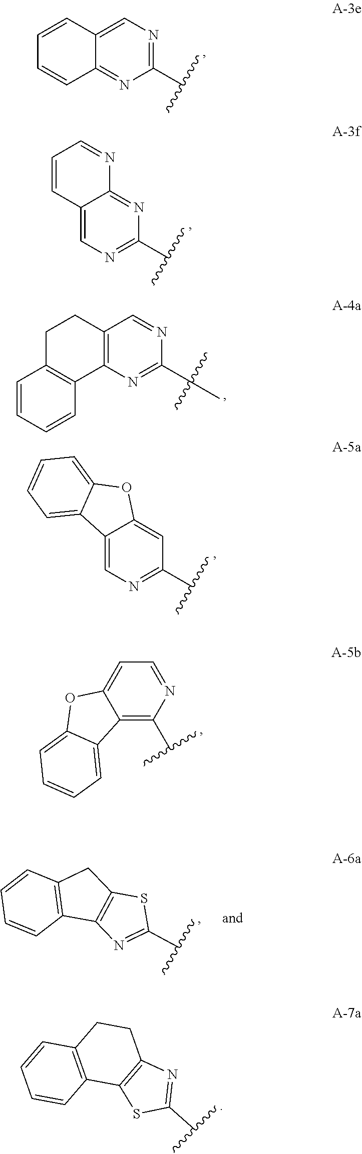

In some embodiments, the substituted Ring A in the compound of Formula I is selected from:

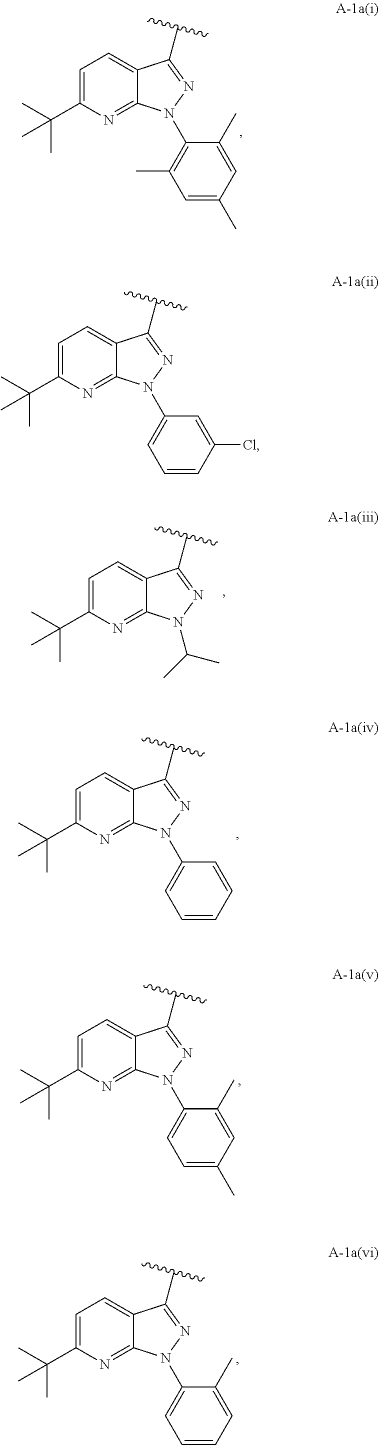

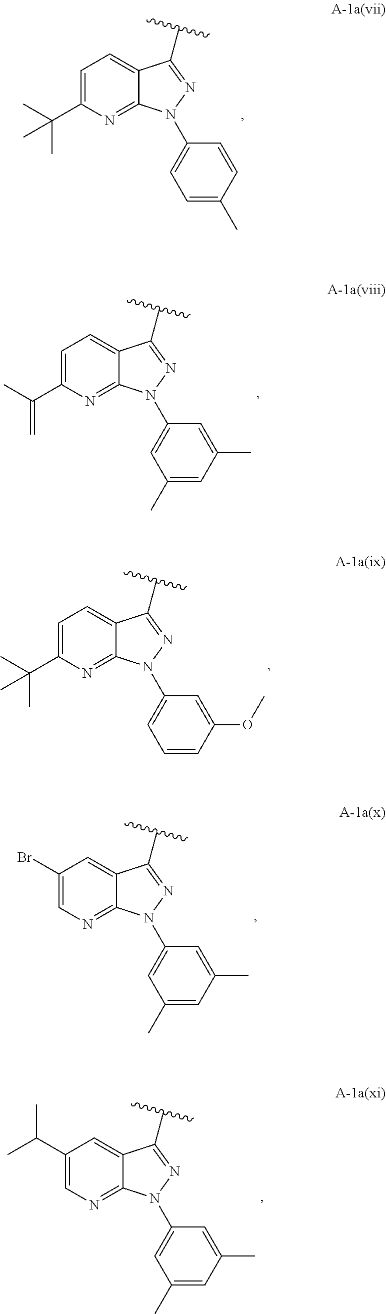

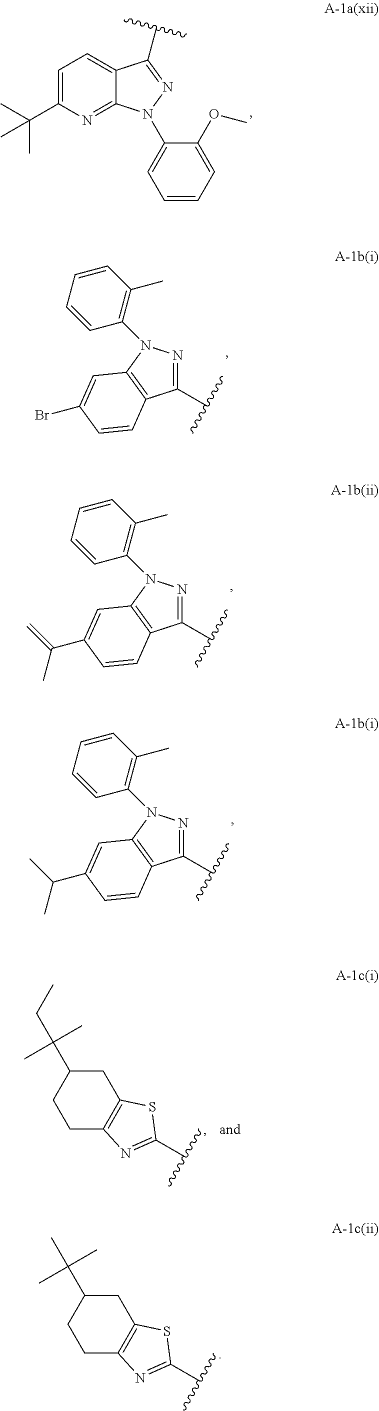

In some embodiments, the substituted Ring A in the compound of Formula I is selected from:

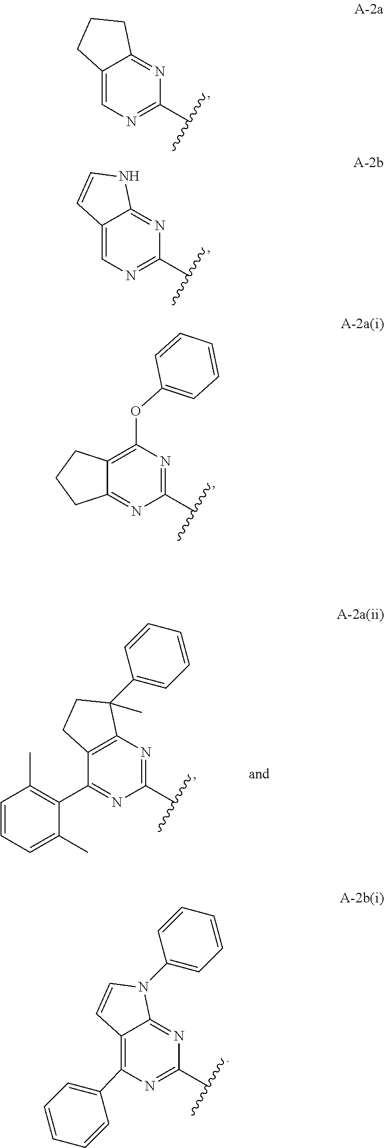 [SMILES: *c1ncc2c(n1)CCC2 ; *c1ncc2cc[nH]c2n1 ; *c1nc2c(c(Oc3ccccc3)n1)CCC2 ; *c1nc(-c2c(C)cccc2C)c2c(n1)C(C)(c1ccccc1)CC2 ; *c1nc(-c2ccccc2)c2ccn(-c3ccccc3)c2n1]

In some embodiments, the substituted Ring A in the compound of Formula I is selected from:

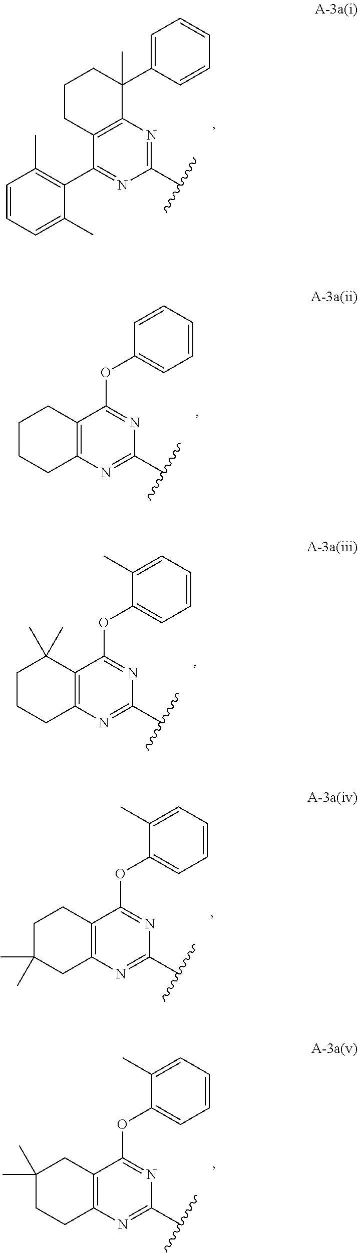 [SMILES: *c1nc(-c2c(C)cccc2C)c2c(n1)C(C)(c1ccccc1)CCC2 ; *c1nc2c(c(Oc3ccccc3)n1)CCCC2 ; *c1nc2c(c(Oc3ccccc3C)n1)C(C)(C)CCC2 ; *c1nc2c(c(Oc3ccccc3C)n1)CCC(C)(C)C2 ; *c1nc2c(c(Oc3ccccc3C)n1)CC(C)(C)CC2]

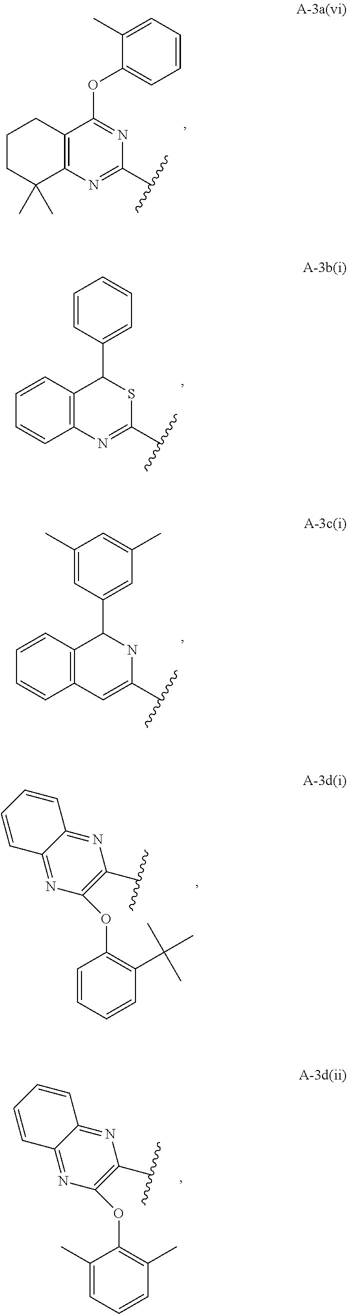 [SMILES: *c1nc(Oc2ccccc2C)c2c(n1)C(C)(C)CCC2 ; *C1=Nc2ccccc2C(c2ccccc2)S1 ; *C1=Cc2ccccc2C(c2cc(C)cc(C)c2)N1 ; *c1nc2ccccc2nc1Oc1ccccc1C(C)(C)C ; *c1nc2ccccc2nc1Oc1c(C)cccc1C]

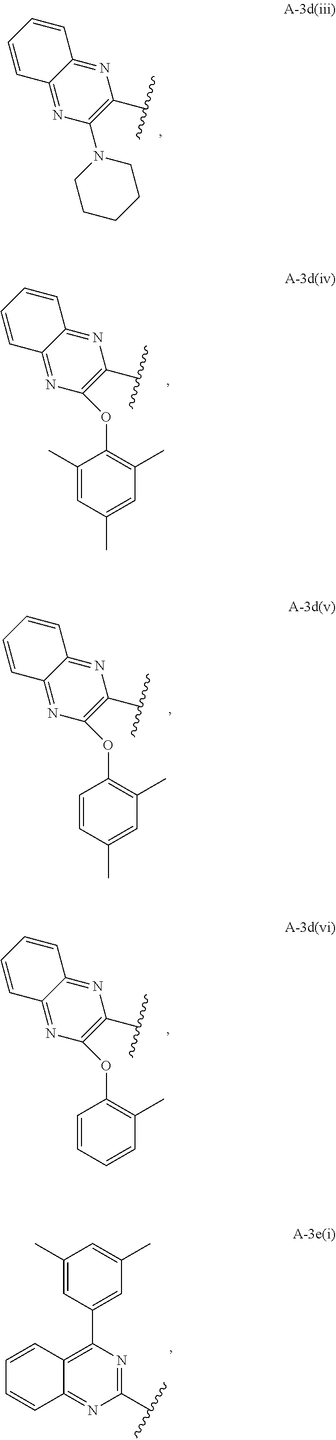 [SMILES: *c1nc2ccccc2nc1N1CCCCC1 ; *c1nc2ccccc2nc1Oc1c(C)cc(C)cc1C ; *c1nc2ccccc2nc1Oc1ccc(C)cc1C ; *c1nc2ccccc2nc1Oc1ccccc1C ; *c1nc(-c2cc(C)cc(C)c2)c2ccccc2n1]

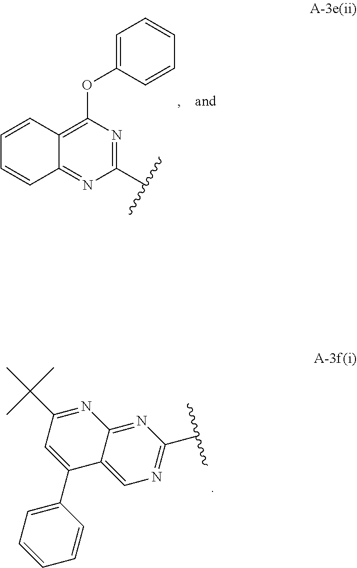 [SMILES: *c1nc(Oc2ccccc2)c2ccccc2n1 ; *c1ncc2c(-c3ccccc3)cc(C(C)(C)C)nc2n1]

In some embodiments, the substituted Ring A in the compound of Formula I is selected from:

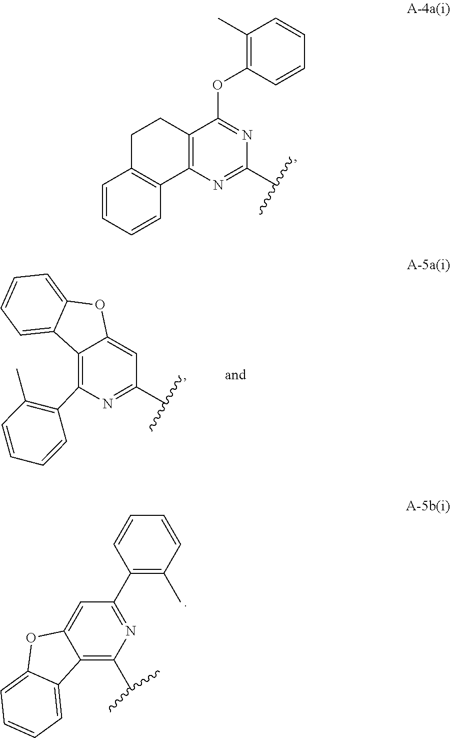 [SMILES: *c1nc(Oc2ccccc2C)c2c(n1)-c1ccccc1CC2 ; *c1cc2oc3ccccc3c2c(-c2ccccc2C)n1 ; *c1nc(-c2ccccc2C)cc2oc3ccccc3c12]

Also disclosed herein are Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing.

### Methods of Treatment

Any of the novel compounds disclosed herein, such as for example, compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as for example, compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, alone or in combination with another active ingredient, such as one or more CFTR modulating agents. In some embodiments, the one or more CFTR modulating agents is a corrector. In some embodiments, the one or more CFTR modulating agents is a potentiator. In some embodiments, the one or more CFTR modulating agents includes both a corrector and a potentiator. In some embodiments, the one or more CFTR modulating agents are selected from potentiators (e.g., ivacaftor, deutivacaftor, and deuterated derivatives and pharmaceutically acceptable salts thereof) and correctors (e.g., lumacaftor, tezacaftor, and deuterated derivatives and pharmaceutically acceptable salts thereof).

In some embodiments, the patient has an F508del/minimal function (MF) genotype, F508del/F508del genotype (homozygous for the F508del mutation), F508del/gating genotype, or F508del/residual function (RF) genotype. In some embodiments, the patient is heterozygous and has one F508del mutation. In some embodiments, the patient is homozygous for the N1303K mutation.

In some embodiments, 5 mg to 500 mg of a compound disclosed herein, a tautomer thereof, a deuterated derivative of the compound and tautomer, or a pharmaceutically acceptable salt of any of the foregoing are administered daily.

In some embodiments, the patient has at least one F508del mutation in the CFTR gene. In some embodiments, the patient has a CFTR gene mutation that is responsive to a compound, tautomer, deuterated derivative, or pharmaceutically acceptable salt of the disclosure based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense mutations | Q2X | L218X | Q525X | R792X | E1104X |
| | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical splice | 185 + 1G→T | 711 + 5G→A | 1717 − 8G→A | 2622 + 1G→A | 3121 − 1G→A |
| mutations | 296 + 1G→A | 712 − 1G→T | 1717 − 1G→A | 2790 − 1G→C | 3500 − 2A→G |
| | 296 + 1G→T | 1248 + 1G→A | 1811 + 1G→C | 3040G→C | 3600 + 2insT |
| | 405 + 1G→A | 1249 − 1G→A | 1811 + 1.6kbA→G | (G970R) | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 nucleotide) | 182delT | 1078delT | 1677delTA | 2711delT | 3737delA |
| insertion/deletion | 306insA | 1119delA | 1782delA | 2732insA | 3791delC |
| (ins/del) frameshift | 306delTAGA | 1138insG | 1824delA | 2869insG | 3821delT |
| mutations | 365-366insT | 1154insTC | 1833delT | 2896insAG | 3876delA |
| | 394delTT | 1161delC | 2043delG | 2942insT | 3878delG |
| | 442delA | 1213delT | 2143delT | 2957delT | 3905insT |
| | 444delA | 1259insA | 2183AA→G^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |

| Non-small (>3 | CFTRdele1 | | CFTRdele16-17b | 1461ins4 |
|---|---|---|---|---|
| nucleotide) | CFTRdele2 | | CFTRdele17a, 17b | 1924del7 |
| insertion/deletion | CFTRdele2,3 | | CFTRdele17a-18 | 2055del9→A |
| (ins/del) frameshift | CFTRdele2-4 | | CFTRdele19 | 2105-2117del13insAGAAA |
| mutations | CFTRdele3-10,14b-16 | | CFTRdele19-21 | 2372del8 |
| | CFTRdele4-7 | | CFTRdele21 | 2721del11 |
| | CFTRdele4-11 | | CFTRdele22-24 | 2991del32 |
| | CFTR50kbdel | | CFTRdele22,23 | 3667ins4 |
| | CFTRdup6b-10 | | 124del23bp | 4010del4 |
| | CFTRdele11 | | 602del14 | 4209TGTT→AA |
| | CFTRdele13,14a | | 852del22 | |
| | CFTRdele14b-17b | | 991del5 | |
| Missense mutations that | A46D | V520F | Y569D | N1303K |
| Are not responsive in | G85E | A559T | L1065P | |
| vitro to TEZ, IVA, or | R347P | R560T | R1066C | |
| TEZ/IVA | L467P | R560S | L1077P | |
| and | I507del | A561E | M1101K | |
| % PI >50% and | | | | |
| SwCl⁻ >86 mmol/L | | | | |

^{a}Also known as 2183delAA→G.
CFTR: cystic fibrosis transmembrane conductance regulator;
IVA: ivacaftor.
SwCl: sweat chloride.
TEZ: tezacaftor.
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.

In some embodiments, the disclosure also is directed to methods of treatment using isotope-labelled compounds of the aforementioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²p, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (2H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salts of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as for example, compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful in methods described herein include Pulmozyme®.

In some embodiments, the additional agent is a bronchodilator. Exemplary bronchodiltors include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreating enzyme replacement), including Pancrease®, Pancreacarb®, Ultrase®, or Creon®, Liprotomase® (formerly Trizytek®), Aquadeks®, or glutathione inhalation. In some embodiments, the additional nutritional agent is pancrelipase.

In some embodiments, at least one additional active pharmaceutical ingredient is selected from CFTR modulating agents. In some embodiments, the active pharmaceutical ingredient is selected from CFTR potentiators. In some embodiments, the CFTR potentiators are selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts of any of the foregoing. In some embodiments, the additional active pharmaceutical ingredient is chosen from CFTR correctors. In some embodiments, the CFTR correctors are selected from lumacaftor, tezacaftor, deuterated derivatives of lumacaftor and tezacaftor, and pharmaceutically acceptable salts of any of the foregoing. In some embodiments, the at least one additional active pharmaceutical ingredient is chosen from (a) tezacaftor, lumacaftor, and deuterated derivatives and pharmaceutically acceptable salts thereof; and (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. Thus, in some embodiments, the combination therapies provided herein comprise (a) a compound selected from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing; (b) at least one compound selected from tezacaftor and pharmaceutically acceptable salts thereof, and (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the combination therapies provided herein comprise (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing; (b) at least one compound selected from lumacaftor and pharmaceutically acceptable salts thereof; and (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

In some embodiments, at least one compound chosen from compounds of compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from ivacaftor and deuterated derivatives and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from deutivacaftor and further deuterated derivatives and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound selected from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof, and at least one compound chosen from ivacaftor and deuterated derivatives and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and pharmaceutically acceptable salts thereof. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in combination with lumacaftor and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered once daily. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered twice daily.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered once daily. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered twice daily.

In some embodiments, (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound selected from tezacaftor and deuterated derivatives and pharmaceutically acceptable salts thereof, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered once daily. In some embodiments, (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound selected from tezacaftor and deuterated derivatives and pharmaceutically acceptable salts thereof, and (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, are administered twice daily.

In some embodiments, (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and (b) at least one compound chosen from lumacaftor and pharmaceutically acceptable salts thereof are administered once daily. In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least one compound chosen from lumacaftor and pharmaceutically acceptable salts thereof are administered twice daily.

In some embodiments, (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and (b) at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, is administered twice daily. In some embodiments, (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and (b) at least one compound chosen from lumacaftor and deuterated derivatives and pharmaceutically acceptable salts thereof, are administered once daily and at least one compound chosen from ivacaftor and deuterated derivatives and pharmaceutically acceptable salts thereof, is administered twice daily.

Compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, in combination with one or more of tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives of and pharmaceutically acceptable salts of tezacaftor, lumacaftor, ivacaftor, deutivacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing) is administered once or twice daily or per day means that said given amount is administered per dosing once or twice daily.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in a first pharmaceutical composition; at least one compound chosen from tezacaftor and pharmaceutically acceptable salts thereof is administered in a second pharmaceutical composition; and at least one compound chosen from ivacaftor and pharmaceutically acceptable salts thereof is administered in a third pharmaceutical composition.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in a first pharmaceutical composition; at least one compound chosen from tezacaftor and pharmaceutically acceptable salts thereof is administered in a second pharmaceutical composition; and at least one compound chosen from deutivacaftor and pharmaceutically acceptable salts thereof is administered in a third pharmaceutical composition.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in a first pharmaceutical composition; at least one compound chosen from tezacaftor and pharmaceutically acceptable salts thereof is administered in a second pharmaceutical composition; and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in a first pharmaceutical composition; at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing is administered in a second pharmaceutical composition; and at least one compound chosen from lumacaftor and pharmaceutically acceptable salts thereof is administered in a third pharmaceutical composition.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, is administered in a first pharmaceutical composition; and at least one compound chosen from tezacaftor and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered in a second pharmaceutical composition. In some embodiments, the second pharmaceutical composition comprises a half of a daily dose of said at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and the other half of the daily dose of said at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing is administered in a third pharmaceutical composition.

In some embodiments, at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing; at least one compound chosen from tezacaftor and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, deuterated derivatives and pharmaceutically acceptable salts of any of the foregoing are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and a second composition comprising only ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or pharmaceutically acceptable salt of any of the foregoing, is administered once daily.

Any suitable pharmaceutical compositions can be used for compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tezacaftor, ivacaftor, deutivacaftor, lumacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and tautomers, deuterated derivatives, and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, incorporated herein by reference.

### Pharmaceutical Compositions

Another aspect of the disclosure provides a pharmaceutical composition comprising at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound chosen from tezacaftor and pharmaceutically acceptable salts thereof, and (c) at least one pharmaceutically acceptable carrier.

In some embodiments, the disclosure provides a pharmaceutical composition comprising (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and (c) at least one pharmaceutically acceptable carrier.

In some embodiments, the disclosure provides a pharmaceutical composition comprising (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from ivacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from deutivacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound chosen from tezacaftor, lumacaftor, and pharmaceutically acceptable salts of tezacaftor and lumacaftor, (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and (d) at least one pharmaceutically acceptable carrier.

In some embodiments, the disclosure provides a pharmaceutical composition comprising (a) at least one compound chosen from compounds of Formula I, Compounds 1-95 (e.g., Compounds 1-7, 9-15, 20, 23, 25-40, 42-48, 50, 51, 53, 55, 56, 58-61, 70, 72, 73, 76, 76, 77, 79), tautomers thereof, deuterated derivatives of those compounds and tautomers, and pharmaceutically acceptable salts of any of the foregoing, (b) at least one compound chosen from lumacaftor and pharmaceutically acceptable salts thereof, (c) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21^{st} edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Exemplary Embodiments

Without limitation, some embodiments of the disclosure include:
1. A compound represented by the following structural formula:

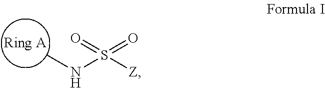

a tautomer thereof, a deuterated derivative of that compound or tautomer, or a pharmaceutically acceptable salt of any of the foregoing, wherein:
Ring A is a bicyclic or tricyclic ring system selected from:

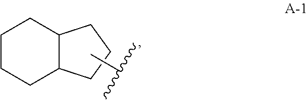

wherein Ring A-1 contains one or more unsaturated bonds and 2 to 3 ring carbon atoms that are independently replaced by nitrogen or sulfur;

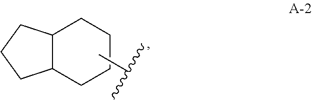

wherein Ring A-2 contains one or more unsaturated bonds and 1 to 3 ring carbon atoms that are replaced by nitrogen;

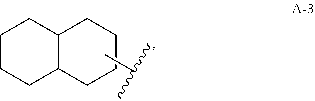

wherein Ring A-3 contains one or more unsaturated bonds and 1 to 3 ring carbon atoms that are independently replaced by nitrogen or sulfur;

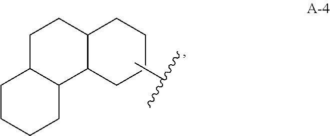

wherein Ring A-4 contains one or more unsaturated bonds and 1 to 3 ring carbon atoms that are replaced by nitrogen;

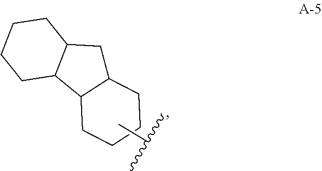

wherein Ring A-5 contains one or more unsaturated bonds and 1 to 3 ring carbon atoms that are independently replaced by nitrogen or oxygen;

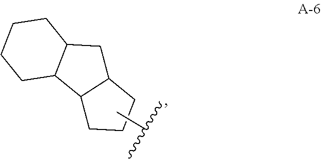

wherein Ring A-6 contains one or more unsaturated bonds and 2 to 3 ring carbon atoms that are independently replaced by nitrogen or sulfur; and

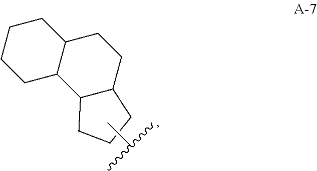

wherein Ring A-7 contains one or more unsaturated bonds and 2 to 3 ring carbon atoms that are independently replaced by nitrogen or sulfur;

Ring A is optionally substituted with 1 to 3 R¹ groups; wherein each R¹ is independently selected from:
halogen,
—C₁-C₆ alkyl optionally substituted with 1 to 3 groups selected from halogen, —OH, and —C₁-C₄ alkoxy;
phenyl optionally substituted with 1 to 3 groups selected from halogen, —CN, —C₁-C₆ alkyl (which is optionally further substituted with 1 to 3 groups selected from halogen and —OH), and —C₁-C₆ alkoxy;
—O-phenyl optionally substituted with 1 to 3 groups selected from halogen, —CN, and —C₁-C₆ alkyl (which is optionally further substituted with 1 to 3 groups selected from halogen and —OH); and
piperidinyl; and
Z is selected from:
phenyl optionally substituted with 1 or 2 groups independently selected from —NH₂ (optionally substituted with 1-2 groups selected from —C₁-C₃ alkyl), —C₁-C₃ alkyl, and —C₁-C₃ alkoxy;
pyrazolyl optionally substituted with 1 or 2 groups independently selected from halogen, —C₁-C₃ alkyl, and —C₁-C₃ alkoxy;
imidazolyl;
1,4 benzodioxanyl; and
pyridinyl optionally substituted with NH₂; provided that the compound is not one of the following:
4-methyl-N-(1-methylisoquinolin-3-yl)benzenesulfonamide;
N-(4,5,6,7-tetrahydro-6-methyl-2-benzothiazolyl)benzenesulfonamide; and
N-[4,5,6,7-tetrahydro-1-(2-pyridinyl)-1H-indazol-4-yl]benzenesulfonamide.
2. The compound, tautomer, deuterated derivative, or pharmaceutically acceptable salt according to embodiment 1, wherein Ring A is selected from:

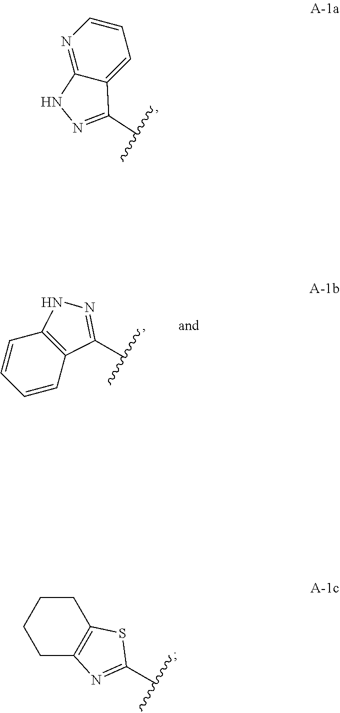 [SMILES: *c1n[nH]c2ncccc12 ; *c1n[nH]c2ccccc12 ; *c1nc2c(s1)CCCC2]

wherein each of A-1a through A-1c is optionally substituted with 1 to 3 R¹ groups as defined in embodiment 1,
and wherein when Ring A is A-1b or A-1c, and Z is phenyl, Z is unsubstituted.
3. The compound, tautomer, deuterated derivative, or pharmaceutically acceptable salt according to embodiment 2, wherein substituted Ring A-1a is selected from:

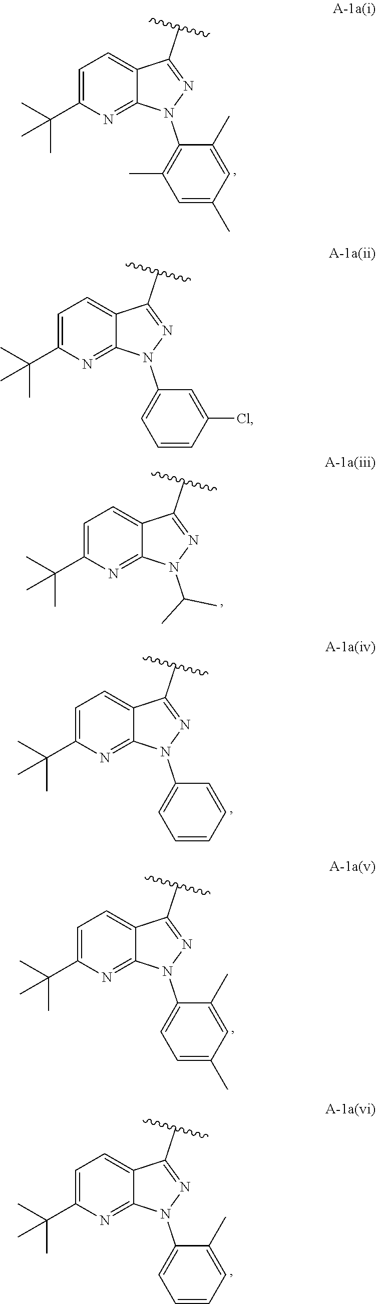

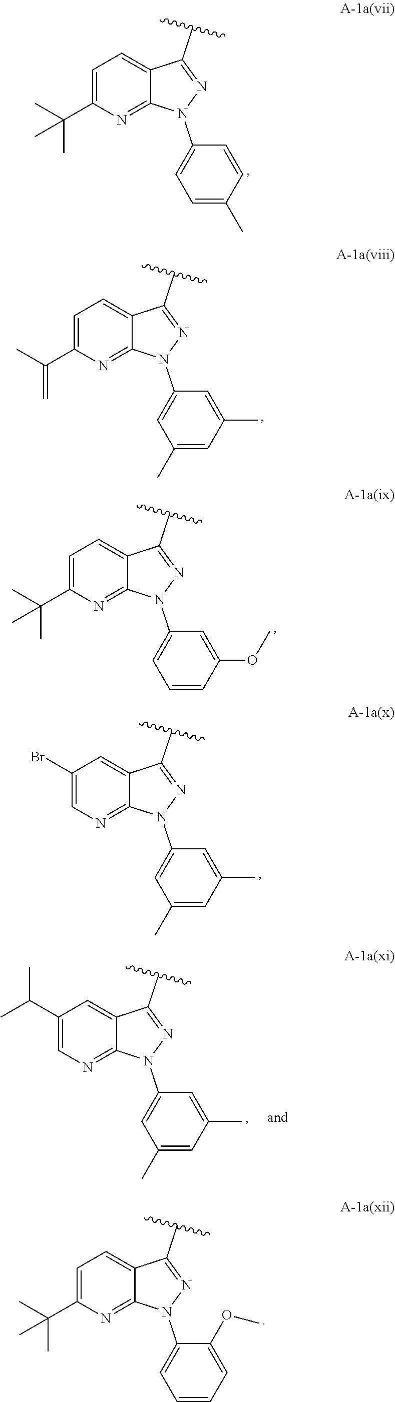

4. The compound, tautomer, deuterated derivative, or pharmaceutically acceptable salt according to embodiment 2, wherein substituted Ring A-1b is selected from:

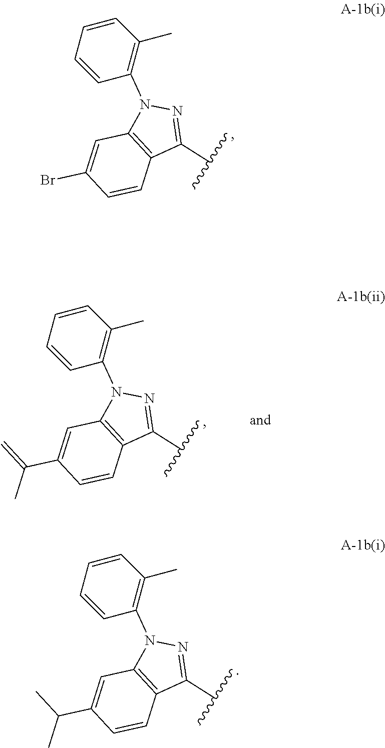 [SMILES: *c1nn(-c2ccccc2C)c2cc(Br)ccc12 ; *c1nn(-c2ccccc2C)c2cc(C(=C)C)ccc12 ; *c1nn(-c2ccccc2C)c2cc(C(C)C)ccc12]

5. The compound, tautomer, deuterated derivative, or pharmaceutically acceptable salt according to embodiment 2, wherein substituted Ring A-1c is selected from:

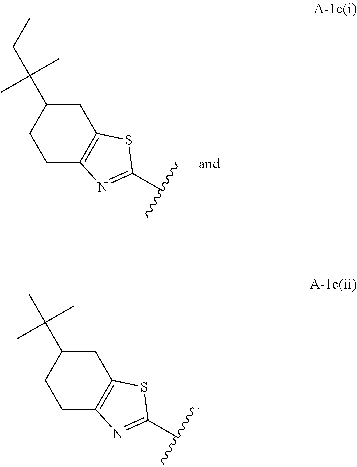 [SMILES: *c1nc2c(s1)CC(C(C)(C)CC)CC2 ; *c1nc2c(s1)CC(C(C)(C)C)CC2]

6. The compound, tautomer, deuterated derivative, or pharmaceutically acceptable salt according to embodiment 1, wherein Ring A is selected from:

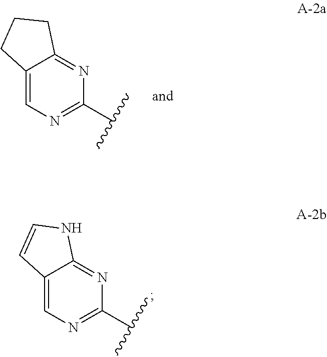 [SMILES: *c1ncc2c(n1)CCC2 ; *c1ncc2cc[nH]c2n1]

wherein each of A-2a and A-2b is optionally substituted with 1 to 3 R¹ groups as defined in embodiment 1,
and wherein when Ring A is A-2a and Z is phenyl, Z is unsubstituted.
7. The compound, tautomer, deuterated derivative, or pharmaceutically acceptable salt according to embodiment 6, wherein substituted Ring A-2a is selected from:

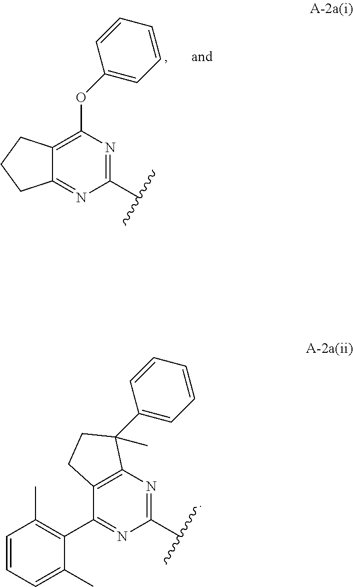 [SMILES: *c1nc2c(c(Oc3ccccc3)n1)CCC2 ; *c1nc(-c2c(C)cccc2C)c2c(n1)C(C)(c1ccccc1)CC2]

8. The compound, tautomer, deuterated derivative, or pharmaceutically acceptable salt according to embodiment 6, wherein substituted Ring A-2b is:

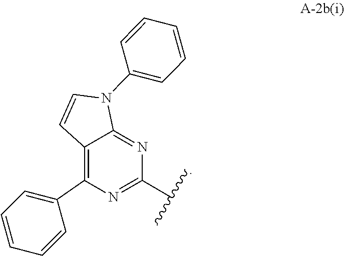 [SMILES: *c1nc(-c2ccccc2)c2ccn(-c3ccccc3)c2n1]

9. The compound, tautomer, deuterated derivative, or pharmaceutically acceptable salt according to embodiment 1, wherein Ring A is selected from:

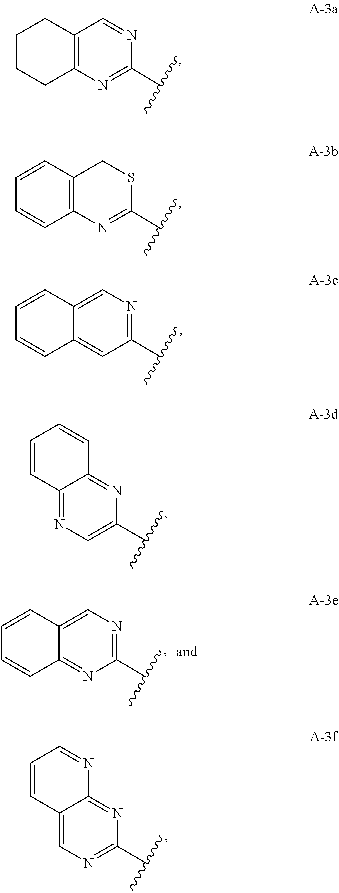 [SMILES: *c1ncc2c(n1)CCCC2 ; *C1=Nc2ccccc2CS1 ; *c1cc2ccccc2cn1 ; *c1cnc2ccccc2n1 ; *c1ncc2ccccc2n1 ; *c1ncc2cccnc2n1]

wherein each of A-3a through A-3f is optionally substituted with 1 to 3 R¹ groups as defined in embodiment 1,
and wherein when Ring A is A-3d or A-3e and Z is phenyl, Z is unsubstituted.
10. The compound, tautomer, deuterated derivative, or pharmaceutically acceptable salt according to embodiment 9, wherein substituted Ring A-3a is selected from:

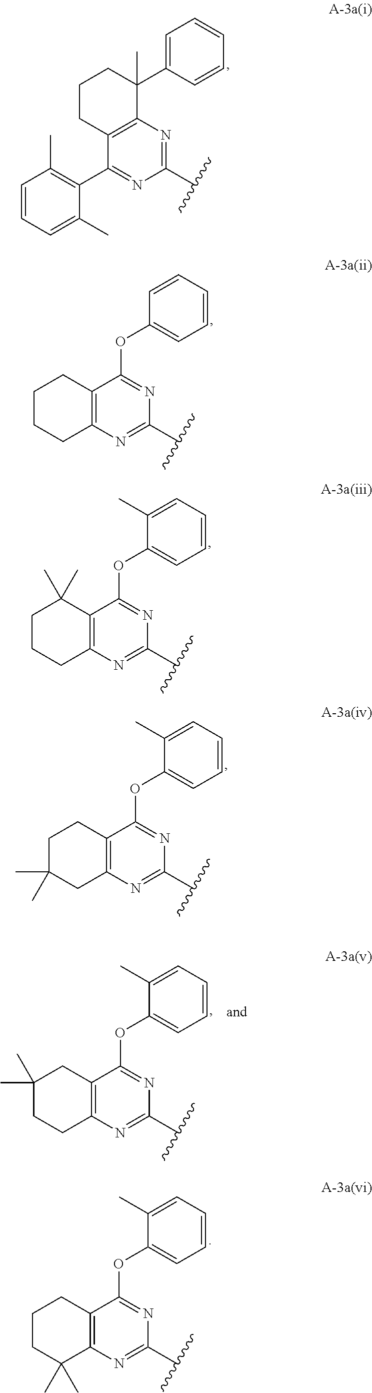 [SMILES: *c1nc(-c2c(C)cccc2C)c2c(n1)C(C)(c1ccccc1)CCC2 ; *c1nc2c(c(Oc3ccccc3)n1)CCCC2 ; *c1nc2c(c(Oc3ccccc3C)n1)C(C)(C)CCC2 ; *c1nc2c(c(Oc3ccccc3C)n1)CCC(C)(C)C2 ; *c1nc2c(c(Oc3ccccc3C)n1)CC(C)(C)CC2 ; *c1nc(Oc2ccccc2C)c2c(n1)C(C)(C)CCC2]

11. The compound, tautomer, deuterated derivative, or pharmaceutically acceptable salt according to embodiment 9, wherein substituted Ring A-3b is:

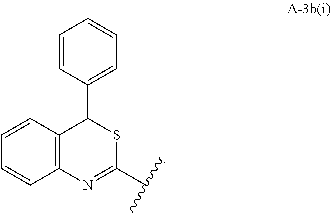 [SMILES: *C1=Nc2ccccc2C(c2ccccc2)S1]

12. The compound, tautomer, deuterated derivative, or pharmaceutically acceptable salt according to embodiment 9, wherein substituted Ring A-3c is:

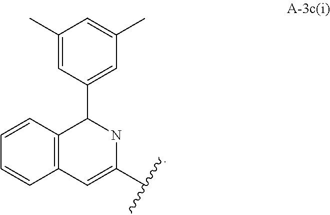 [SMILES: *C1=Cc2ccccc2C(c2cc(C)cc(C)c2)N1]

13. The compound, tautomer, deuterated derivative, or pharmaceutically acceptable salt according to embodiment 9, wherein substituted Ring A-3d is selected from:

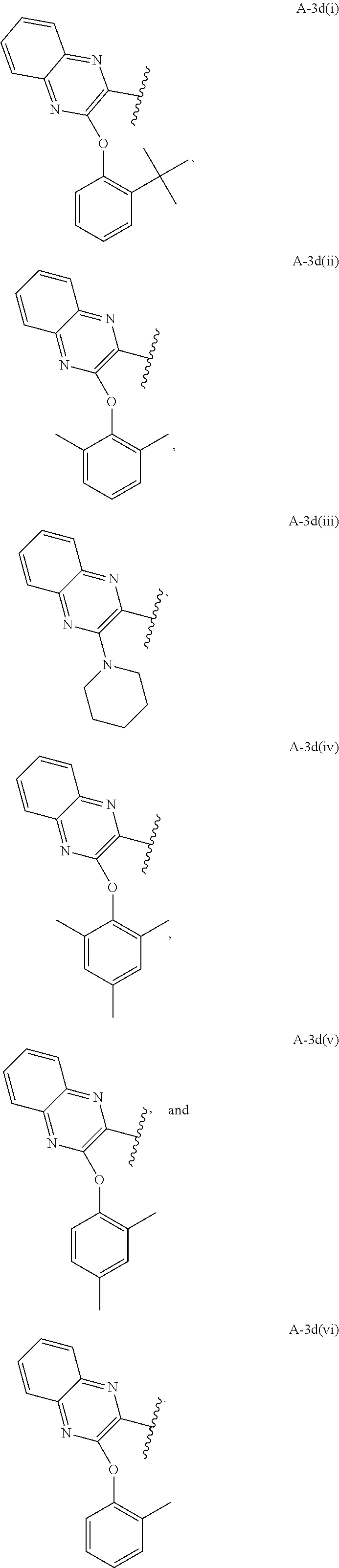 [SMILES: *c1nc2ccccc2nc1Oc1ccccc1C(C)(C)C ; *c1nc2ccccc2nc1Oc1c(C)cccc1C ; *c1nc2ccccc2nc1N1CCCCC1 ; *c1nc2ccccc2nc1Oc1c(C)cc(C)cc1C ; *c1nc2ccccc2nc1Oc1ccc(C)cc1C ; *c1nc2ccccc2nc1Oc1ccccc1C]

14. The compound, tautomer, deuterated derivative, or pharmaceutically acceptable salt according to embodiment 9, wherein substituted Ring A-3e is selected from:

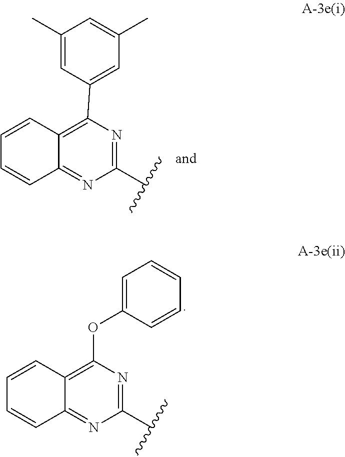 [SMILES: *c1nc(-c2cc(C)cc(C)c2)c2ccccc2n1 ; *c1nc(Oc2ccccc2)c2ccccc2n1]

15. The compound, tautomer, deuterated derivative, or pharmaceutically acceptable salt according to embodiment 9, wherein substituted Ring A-3f is:

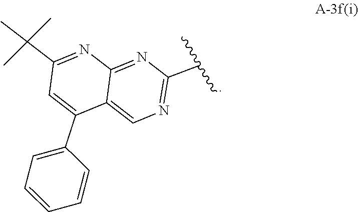 [SMILES: *c1ncc2c(-c3ccccc3)cc(C(C)(C)C)nc2n1]

16. The compound, tautomer, deuterated derivative, or pharmaceutically acceptable salt according to embodiment 1, wherein Ring A is:

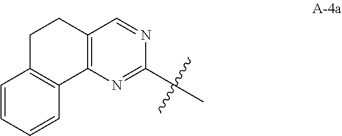 [SMILES: *c1ncc2c(n1)-c1ccccc1CC2]

optionally substituted with 1 to 3 R¹ groups as defined in embodiment 1.
17. The compound, tautomer, deuterated derivative, or pharmaceutically acceptable salt according to embodiment 15, wherein Ring A-4a is:

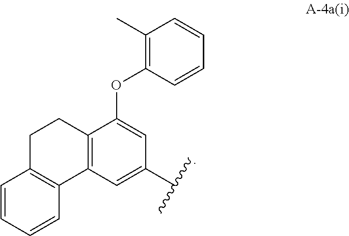 [SMILES: *c1cc(Oc2ccccc2C)c2c(c1)-c1ccccc1CC2]

18. The compound, tautomer, deuterated derivative, or pharmaceutically acceptable salt according to embodiment 1, wherein Ring A is selected from:

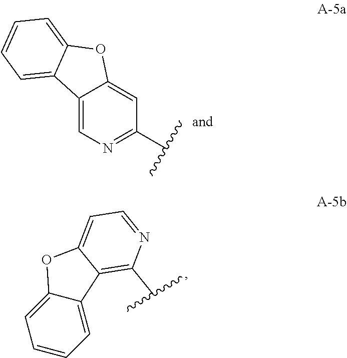 [SMILES: *c1cc2oc3ccccc3c2cn1 ; *c1nccc2oc3ccccc3c12]

wherein each of A-5a and A-5b is optionally substituted with 1 to 3 R¹ groups as defined in embodiment 1.
19. The compound, tautomer, deuterated derivative, or pharmaceutically acceptable salt according to embodiment 18, wherein Ring A-5a is:

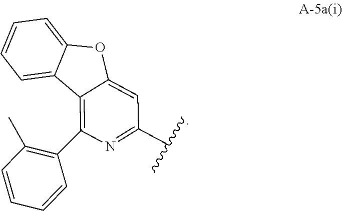 [SMILES: *c1cc2oc3ccccc3c2c(-c2ccccc2C)n1]

20. The compound, tautomer, deuterated derivative, or pharmaceutically acceptable salt according to embodiment 18, wherein Ring A-5b is:

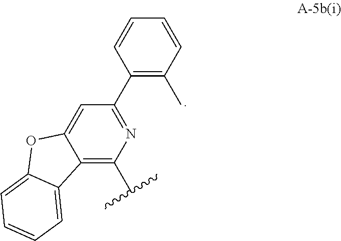 [SMILES: *c1nc(-c2ccccc2C)cc2oc3ccccc3c12]

21. The compound, tautomer, deuterated derivative, or pharmaceutically acceptable salt according to embodiment 1, wherein Ring A is:

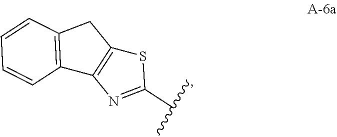 [SMILES: *c1nc2c(s1)Cc1ccccc1-2]

optionally substituted with 1 to 3 R¹ groups.
22. The compound, tautomer, deuterated derivative, or pharmaceutically acceptable salt according to embodiment 1, wherein Ring A is:

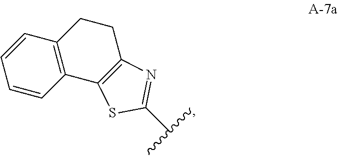 [SMILES: *c1nc2c(s1)-c1ccccc1CC2]

optionally substituted with 1 to 3 R¹ groups.
23. The compound, tautomer, deuterated derivative, or pharmaceutically acceptable salt according to any one of embodiments 1-22, wherein Z is optionally substituted phenyl.
24. The compound, tautomer, deuterated derivative, or pharmaceutically acceptable salt according to any one of embodiments 1-22, wherein Z is phenyl substituted with substituted with 1-2 groups independently selected from NH₂, OCH₃, and N(CH₃)₂.
25. The compound, tautomer, deuterated derivative, or pharmaceutically acceptable salt according to any one of embodiments 1-22, wherein Z is pyrazolyl optionally substituted with CH₃.
26. A compound selected from Compounds 1-95 (e.g., Compounds 1-7, 9-15, 20, 23, 25-40, 42-48, 50, 51, 53, 55, 56, 58-61, 70, 72, 73, 76, 76, 77, 79), or a deuterated derivative or pharmaceutically acceptable salt thereof.
27. A pharmaceutical composition comprising a compound, tautomer, deuterated derivative, or pharmaceutically acceptable salt of any one of embodiments 1-26 and a pharmaceutically acceptable carrier.
28. The pharmaceutical composition of embodiment 27, further comprising one or more additional therapeutic agent(s).
29. The pharmaceutical composition of embodiment 28, wherein the one or more additional therapeutic agent(s) comprise(s) a compound selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.
30. The pharmaceutical composition of embodiment 29, wherein the composition comprises (a) a compound, tautomer, deuterated derivative, or pharmaceutically acceptable salt of any one of embodiments 1-26, (b) a compound selected from tezacaftor and pharmaceutically acceptable salts thereof, (c) a compound selected from ivacaftor and pharmaceutically acceptable salts thereof, and (d) a pharmaceutically acceptable carrier.
31. The pharmaceutical composition of embodiment 29, wherein the composition comprises (a) a compound, tautomer, deuterated derivative, or pharmaceutically acceptable salt of any one of embodiments 1-26, (b) a compound selected from tezacaftor and pharmaceutically acceptable salts thereof, (c) a compound selected from deutivacaftor and pharmaceutically acceptable salts thereof, and (d) a pharmaceutically acceptable carrier.
32. The pharmaceutical composition of embodiment 29, wherein the composition comprises (a) a compound, tautomer, deuterated derivative, or pharmaceutically acceptable salt of any one of embodiments 1-26, (b) a compound selected from tezacaftor and pharmaceutically acceptable salts thereof, (c) a compound selected from (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing, and (d) a pharmaceutically acceptable carrier.
33. The pharmaceutical composition of embodiment 29, wherein the composition comprises (a) a compound, tautomer, deuterated derivative, or pharmaceutically acceptable salt of any one of embodiments 1-26, (b) a compound selected from lumacaftor and pharmaceutically acceptable salts thereof, (c) a compound selected from ivacaftor and pharmaceutically acceptable salts thereof, and (d) a pharmaceutically acceptable carrier.
34. The pharmaceutical composition of embodiment 29, wherein the composition comprises (a) a compound, tautomer, deuterated derivative, or pharmaceutically acceptable salt of any one of embodiments 1-26, (b) a compound selected from lumacaftor and pharmaceutically acceptable salts thereof, (c) a compound selected from deutivacaftor and pharmaceutically acceptable salts thereof, and (d) a pharmaceutically acceptable carrier.
35. The pharmaceutical composition of embodiment 29, wherein the composition comprises (a) a compound, tautomer, deuterated derivative, or pharmaceutically acceptable salt of any one of embodiments 1-26, (b) a compound selected from lumacaftor and pharmaceutically acceptable salts thereof, (c) a compound selected from (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing, and (d) a pharmaceutically acceptable carrier.
36. The pharmaceutical composition of embodiment 29, wherein the composition comprises (a) a compound, tautomer, deuterated derivative, or pharmaceutically acceptable salt of any one of embodiments 1-26, and (b) a compound selected from ivacaftor, lumacaftor, (6R,12R)-17-amino-12-methyl-6,15-bis(trifluoromethyl)-13,19-dioxa-3,4,18-triazatricyclo [12.3.1.12,5]nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and pharmaceutically acceptable salts of any of the foregoing, and (c) a pharmaceutically acceptable carrier.
37. A pharmaceutical composition comprising:
   (a) at least one compound, tautomer, deuterated derivative, or pharmaceutically acceptable salt according to any one of embodiments 1-26;
   (b) at least one pharmaceutically acceptable carrier; and optionally one or more of:
   (i) a compound chosen from tezacaftor:

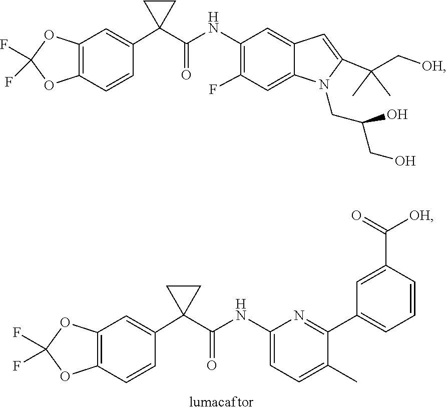 [SMILES: CC(C)(CO)c1cc2cc(NC(=O)C3(c4ccc5c(c4)OC(F)(F)O5)CC3)c(F)cc2n1C[C@@H](O)CO ; Cc1ccc(NC(=O)C2(c3ccc4c(c3)OC(F)(F)O4)CC2)nc1-c1cccc(C(=O)O)c1]

and pharmaceutically acceptable salts and deuterated derivatives thereof; and
   (ii) a compound chosen from

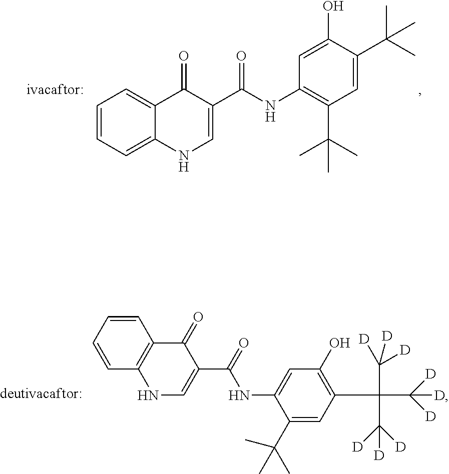

(6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol:

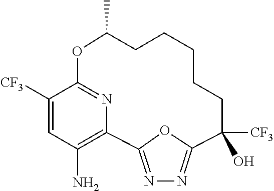 [SMILES: C[C@@H]1CCCCC[C@](O)(C(F)(F)F)c2nnc(o2)-c2nc(c(C(F)(F)F)cc2N)O1]

and deuterated derivatives and pharmaceutically acceptable salts thereof.
38. A method of treating cystic fibrosis comprising administering to a patient in need thereof a compound, tautomer, deuterated derivative, or pharmaceutically acceptable salt of any one of embodiments 1-26 or a pharmaceutical composition according to any one of embodiments 27-37.
39. The method of embodiment 38, further comprising administering to the patient one or more additional therapeutic agent(s) prior to, concurrent with, or subsequent to the compound or the pharmaceutical composition.
40. The method of embodiment 39, wherein the one or more additional therapeutic agent(s) comprise(s) a compound selected from tezacaftor, lumacaftor, ivacaftor, deutivacaftor, lumacaftor, (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.
41. The method of embodiment 40, wherein the additional therapeutic agents comprise tezacaftor and ivacaftor.
42. The method of embodiment 40, wherein the additional therapeutic agents comprise tezacaftor and deutivacaftor.
43. The method of embodiment 40, wherein the additional therapeutic agents comprise tezacaftor and a compound selected from (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.
44. The method of embodiment 40, wherein the therapeutic agent is selected from ivacaftor, deutivacaftor, (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.
45. The method of embodiment 40, wherein the additional therapeutic agents comprise lumacaftor and ivacaftor.
46. The method of embodiment 40, wherein the additional therapeutic agents comprise lumacaftor and deutivacaftor.
47. The method of embodiment 40, wherein the additional therapeutic agents comprise lumacaftor and a compound selected from (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.
48. The compound, tautomer, deuterated derivative, or pharmaceutically acceptable salt of any one of embodiments 1-26 or the pharmaceutical composition according to any one of embodiments 27-37 for use in the treatment of cystic fibrosis.
49. The compound, tautomer, deuterated derivative, or pharmaceutically acceptable salt of any one of embodiments 1-26 or the pharmaceutical composition according to any one of embodiments 27-37 for use in the manufacture of a medicament for the treatment of cystic fibrosis.

### Examples

### I. Abbreviation List

API: Active pharmaceutical ingredient
ACN: Acetonitrile
Boc anhydride ((Boc)₂O): Di-tert-butyl dicarbonate
CDCl₃: Chloroform-d CDI: Carbonyl diimidazole
CDMT: 2-Chloro-4,6-dimethoxy-1,3,5-triazine
CH₂Cl₂: Dichloromethane
CH₃CN: Acetonitrile
COMU: (1-Cyano-2-ethoxy-2-oxoethylidenaminooxy)dimethylamino-morpholino-carbenium hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
ESI-MS: Electrospray ionization mass spectrometry
EtOAc: Ethyl acetate
EtOH: Ethanol
GC: Gas chromatography
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
Hoveyda-Grubbs 2^{nd} Generation catalyst: (1,3-Lcis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
HPLC: High-performance liquid chromatography
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MgSO₄: Magnesium sulfate
MTBE: Methyl tert-butyl ether
MeTHF or 2-MeTHF: 2-Methyltetrahydrofuran
NaHCO₃: Sodium bicarbonate
NaOH: Sodium hydroxide
NMP: N-Methyl-2-pyrrolidone
NMM: N-Methylmorpholine
NMR: Nuclear magnetic resonance
Pd/C: Palladium on carbon
Pd₂(dba)₃: Tris(dibenzylideneacetone)dipalladium(O)
Pd(dppf)Cl₂: [1,1′-Bis(diphenylphosphino)ferrocene]dichloropalladium(II)
Pd(Oac)₂: Palladium(II) acetate
PTFE: Polytetrafluoroethylene
rt, RT: Room temperature
RuPhos: 2-Dicyclohexylphosphino-2′,6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
T3P: Propanephosphonic acid anhydride
UPLC: Ultra Performance Liquid Chromatography
XANTPHOS: 4,5-Bis(diphenylphosphino)-9,9-dimethylxanthene
Xphos: 2-Dicyclohexylphosphino-2′,4′,6′-triisopropylbiphenyl

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### III. General UPLC/HPLC/GC Analytical Methods

LC method A: Analytical reverse phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method C: Kinetex C₁₈ 4.6×50 mm 2.6 μm. Temp: 45° C., Flow: 2.0 mL/min, Run Time: 3 min. Mobile phase: Initial 95% water (0.1% formic acid) and 5% acetonitrile (0.1% formic acid) linear gradient to 95% acetonitrile (0.1% formic acid) for 2.0 min then hold at 95% acetonitrile (0.1% formic acid) for 1.0 min.

LC method D: Acquity UPLC BEH C₁₈ column (30×2.1 mm, 1.7 μm particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC method I: Acquity UPLC BEH C₁₈ column (50×2.1 mm, 1.7 μm particle) made by Waters (pn:186002350), and a dual gradient run from 1-99% mobile phase B over 5.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method J: Reverse phase UPLC using an Acquity UPLC BEH C₁₈ column (50×2.1 mm, 1.7 μm particle) made by Waters (pn: 186002350), and a dual gradient run from 1-99% mobile phase B over 2.9 minutes. Mobile phase A=H₂O (0.05% NH₄HCO₂). Mobile phase B=CH₃CN. Flow rate=1.2 mL/min, injection volume=1.5 μL, and column temperature=60° C.

LC method K: Kinetex Polar C₁₈ 3.0×50 mm 2.6 μm, 3 min, 5-95% ACN in H₂O (0.1% Formic Acid) 1.2 ml/min.

LC method Q: Reversed phase UPLC using an Acquity UPLC BEH C₁₈ column (50×2.1 mm, 1.7 μm particle) made by Waters (pn: 186002350), and a dual gradient run from 30-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method S: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method T: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method U: Kinetex Polar C₁₈ 3.0×50 mm 2.6 μm, 6 min, 5-95% ACN in H₂O (0.1% Formic Acid) 1.2 mL/min.

LC method V: Acquity UPLC BEH C₁₈ column (50×2.1 mm, 1.7 μm particle) made by Waters (pn: 186002350), and a dual gradient run from 1-30% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method W: water Cortex 2.7 μ C₁₈ (3.0 mm×50 mm), Temp: 55° C.; Flow: 1.2 mL/min; mobile phase: 100% water with 0.1% trifluoroacetic(TFA) acid then 100% acetonitrile with 0.1% TFA acid, grad:5% to 100% B over 4 min, with stay at 100% B for 0.5 min, equilibration to 5% B over 1.5 min.

LC method X: UPLC Luna C₁₈(2) 50×3 mm 3 m. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA, T: 45C, Flow: 1.5 mL/min

LC method Y: UPLC SunFire C₁₈ 75×4.6 mm 3.5 m, run: 6 min. Mobile phase conditions: Initial 95% H₂O+0.1% FA/5% CH₃CN+0.1% FA, linear gradient to 95% CH₃CN for 4 min, hold for 2 min at 95% CH₃CN. T:45° C., Flow:1.5 mL/min

### IV. Synthesis of Compounds

### Compound 1

### N-[5,5-dimethyl-4-(2-methylphenoxy)-7,8-dihydro-6H-quinazolin-2-yl]-1-methyl-pyrazole-4-sulfonamide

### Step 1: 5,5-Dimethyl-4-(2-methylphenoxy)-7,8-dihydro-6H-quinazolin-2-amine

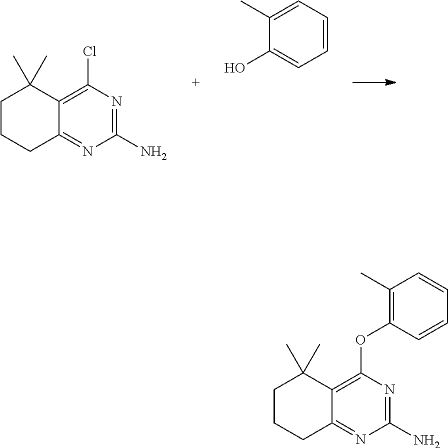

To 4-chloro-5,5-dimethyl-7,8-dihydro-6H-quinazolin-2-amine (150 mg, 0.7086 mmol) and potassium carbonate (315.4 mg, 2.282 mmol) was added DMF (1.7 mL) followed by o-cresol (139 μL, 0.7108 mmol). The mixture was heated at 130° C. for 118 h. EtOAc and water were added to the reaction. The two layers were separated after shaking. The aqueous layer was extracted with EtOAc (×1). The combined organic layer was dried over Na₂SO₄, filtered and concentrated. The crude product was purified on 40 g of silica gel utilizing a gradient of 0-100% ethyl acetate in hexane to yield 5,5-dimethyl-4-(2-methylphenoxy)-7,8-dihydro-6H-quinazolin-2-amine (140.1 mg, 70%). ESI-MS m/z calc. 283.16846, found 284.6 (M+1)⁺; Retention time: 1.33 minutes; LC method A.

### Step 2: N-[5,5-dimethyl-4-(2-methylphenoxy)-7,8-dihydro-6H-quinazolin-2-yl]-1-methyl-pyrazole-4-sulfonamide

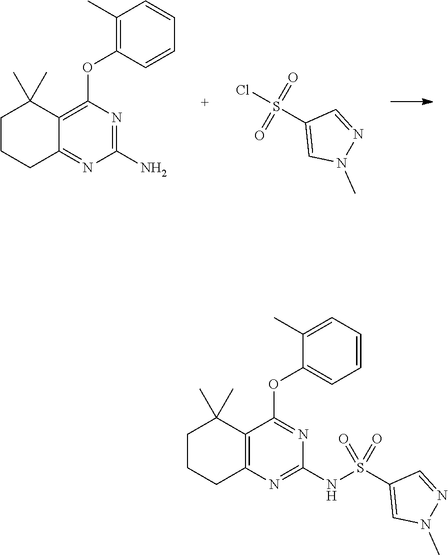

To 5,5-dimethyl-4-(2-methylphenoxy)-7,8-dihydro-6H-quinazolin-2-amine (100 mg, 0.3529 mmol), 1-methylpyrazole-4-sulfonyl chloride (250 mg, 1.4 mmol) and pyridine (5.4 mL) were added and the reaction was stirred at 110° C. for 23 h. Water and EtOAc were added to the reaction and the two layers were separated. The organic layer was washed with water (×2), dried over Na₂SO₄, filtered and the solvent was evaporated under reduced pressure. The crude product was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B=CH₃CN) to yield N-[5,5-dimethyl-4-(2-methylphenoxy)-7,8-dihydro-6H-quinazolin-2-yl]-1-methyl-pyrazole-4-sulfonamide (59.8 mg, 40%) as a light brown solid. ¹H NMR (400 MHz, DMSO-d₆) δ 7.42 (dd, J=7.6, 1.8 Hz, 1H), 7.39-7.33 (m, 1H), 7.32-7.25 (m, 1H), 7.10 (dd, J=7.9, 1.3 Hz, 1H), 7.05 (s, 1H), 6.96 (s, 1H), 3.70 (s, 3H), 2.62 (t, J=6.2 Hz, 2H), 2.12 (s, 3H), 1.78-1.69 (m, 2H), 1.69-1.59 (m, 2H), 1.39 (s, 6H). ESI-MS m/z calc. 427.16782, found 428.5 (M+1)⁺; Retention time: 1.59 minutes; LC method A.

### Compound 2

### N-[7,7-Dimethyl-4-(2-methylphenoxy)-6,8-dihydro-5H-quinazolin-2-yl]-1-methyl-pyrazole-4-sulfonamide

### Step 1: 7,7-Dimethyl-4-(2-methylphenoxy)-6,8-dihydro-5H-quinazolin-2-amine

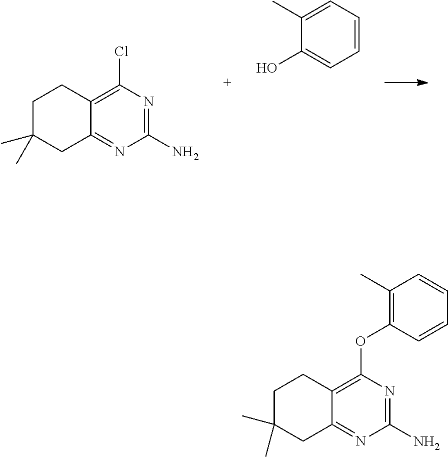

To 4-chloro-7,7-dimethyl-6,8-dihydro-5H-quinazolin-2-amine (152.3 mg, 0.7194 mmol) and potassium carbonate (312.5 mg, 2.261 mmol) was added DMF (1.7 mL) followed by o-cresol (141 μL, 0.7211 mmol). The mixture was heated at 130° C. for 21 h. EtOAc and water were added to the reaction. The two layers were separated after shaking. The aqueous layer was extracted with EtOAc (×1). The organic layer was dried over Na₂SO₄, filtered and concentrated. The crude product was purified on 40 g of silica gel utilizing a gradient of 0-100% ethyl acetate in hexane to yield 7,7-dimethyl-4-(2-methylphenoxy)-6,8-dihydro-5H-quinazolin-2-amine (146.2 mg, 72%) as a yellow solid. ESI-MS m/z calc. 283.16846, found 284.2 (M+1)⁺; Retention time: 1.34 minutes; LC method A.

### Step 2: N-[7,7-Dimethyl-4-(2-methylphenoxy)-6,8-dihydro-5H-quinazolin-2-yl]-1-methyl-pyrazole-4-sulfonamide

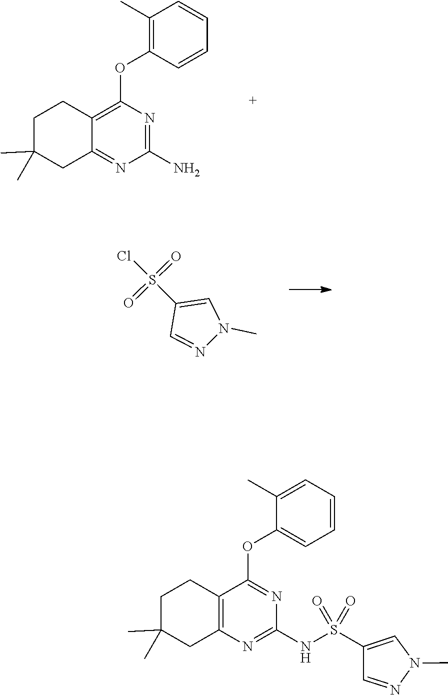

To 7,7-dimethyl-4-(2-methylphenoxy)-6,8-dihydro-5H-quinazolin-2-amine (80 mg, 0.2823 mmol), 1-methylpyrazole-4-sulfonyl chloride (202 mg, 1.118 mmol) and pyridine (4.3 mL) were added and the reaction was stirred at 110° C. for 21 h. Water and EtOAc were added to the reaction and the two layers were separated. The organic layer was washed with water (×2), dried over Na₂SO₄, filtered and the solvent was evaporated under reduced pressure. It was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B=CH₃CN) to yield N-[7,7-dimethyl-4-(2-methylphenoxy)-6,8-dihydro-5H-quinazolin-2-yl]-1-methyl-pyrazole-4-sulfonamide (62.2 mg, 52%) as a white solid. ¹H NMR (400 MHz, DMSO-d₆) δ 7.41 (dd, J=7.6, 1.8 Hz, 1H), 7.36 (td, J=7.7, 2.0 Hz, 1H), 7.29 (td, J 7.4, 1.5 Hz, 1H), 7.19 (dd, J=7.8, 1.4 Hz, 1H), 7.10 (s, 1H), 6.99 (s, 1H), 3.69 (s, 3H), 2.61 (t, J=6.5 Hz, 2H), 2.42 (s, 2H), 2.10 (s, 3H), 1.57 (t, J=6.6 Hz, 2H), 0.98 (s, 6H). ESI-MS m/z calc. 427.16782, found 428.4 (M+1)⁺; Retention time: 1.61 minutes; LC method A.

### Compound 3

### N-[6,6-Dimethyl-4-(2-methylphenoxy)-7,8-dihydro-5H-quinazolin-2-yl]-1-methyl-pyrazole-4-sulfonamide

### Step 1: 6,6-Dimethyl-4-(2-methylphenoxy)-7,8-dihydro-5H-quinazolin-2-amine

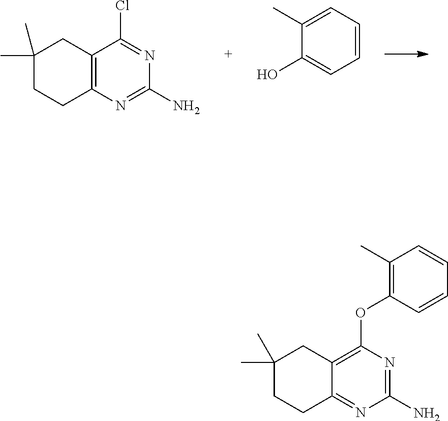

To 4-chloro-6,6-dimethyl-7,8-dihydro-5H-quinazolin-2-amine (150 mg, 0.7086 mmol) and potassium carbonate (316.8 mg, 2.292 mmol) was added DMF (1.700 mL) followed by o-cresol (139 μL, 0.7108 mmol). The mixture was heated at 130° C. for 19 h. EtOAc and water were added to the reaction. The two layers were separated after shaking. The aqueous layer was extracted with EtOAc (×1). The organic layer was dried over Na₂SO₄, filtered and concentrated. The crude product was purified on 40 g of silica gel utilizing a gradient of 0-100% ethyl acetate in hexane to yield 6,6-dimethyl-4-(2-methylphenoxy)-7,8-dihydro-5H-quinazolin-2-amine (131.2 mg, 65%) as a yellow solid. ESI-MS m/z calc. 283.16846, found 284.6 (M+1)⁺; Retention time: 1.34 minutes; LC method A.

### Step 2: N-[6,6-Dimethyl-4-(2-methylphenoxy)-7,8-dihydro-5H-quinazolin-2-yl]-1-methyl-pyrazole-4-sulfonamide

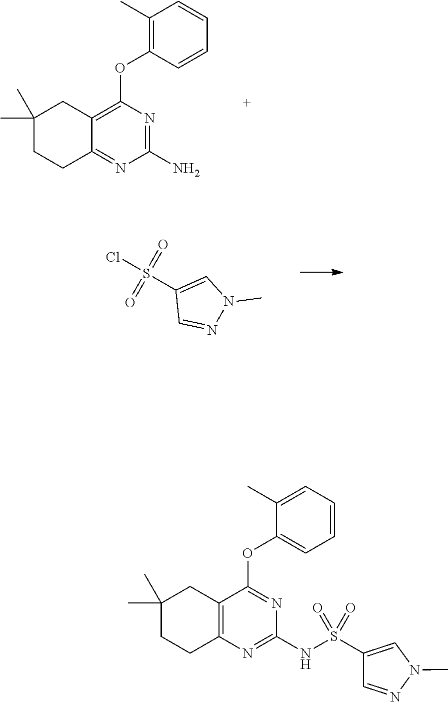

To 6,6-dimethyl-4-(2-methylphenoxy)-7,8-dihydro-5H-quinazolin-2-amine (80 mg, 0.2823 mmol), 1-methylpyrazole-4-sulfonyl chloride (207.3 mg, 1.148 mmol) and pyridine (4.3 mL) were added and the reaction was stirred at 110° C. for 19 h. Water and EtOAc were added to the reaction and the two layers were separated. The organic layer was washed with water (×2), dried over Na₂SO₄, filtered and the solvent was evaporated under reduced pressure. The crude product was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B=CH₃CN). The product was impure. It was redissolved in DMSO, filtered and purified using a reverse phase HPLC C₁₈ column and a dual gradient run from 1-99% mobile phase B over 30 minutes (Mobile phase A=H₂O (5 mM HCl). Mobile phase B=CH₃CN) to yield N-[6,6-dimethyl-4-(2-methylphenoxy)-7,8-dihydro-5H-quinazolin-2-yl]-1-methyl-pyrazole-4-sulfonamide (70.7 mg, 59%) as a white solid. ¹H NMR (400 MHz, DMSO-d6) δ 7.40 (dd, J=7.4, 1.8 Hz, 1H), 7.38-7.32 (m, 1H), 7.31-7.26 (m, 1H), 7.16 (dd, J=8.0, 1.4 Hz, 1H), 7.10 (s, 1H), 6.99 (s, 1H), 3.69 (s, 3H), 2.64 (t, J=6.6 Hz, 2H), 2.40 (s, 2H), 2.08 (s, 3H), 1.57 (t, J=6.6 Hz, 2H), 1.00 (s, 6H). ESI-MS m/z calc. 427.16782, found 428.2 (M+1)⁺; Retention time: 1.61 minutes; LC method A.

### Compound 4

### N-(4-phenoxy-5,6,7,8-tetrahydroquinazolin-2-yl)benzenesulfonamide

### Step 1: N-(4-chloro-5,6,7,8-tetrahydroquinazolin-2-yl)benzenesulfonamide

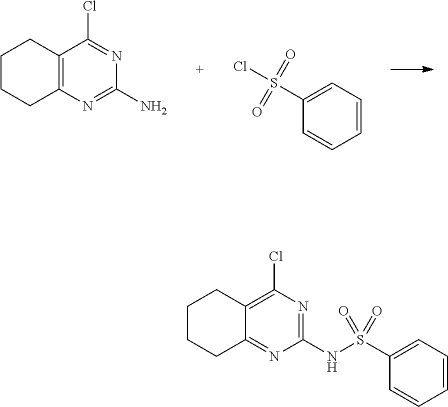 [SMILES: Nc1nc(Cl)c2c(n1)CCCC2 ; O=S(=O)(Cl)c1ccccc1 ; O=S(=O)(Nc1nc(Cl)c2c(n1)CCCC2)c1ccccc1]

To 4-chloro-5,6,7,8-tetrahydroquinazolin-2-amine (1 g, 5.445 mmol) in DMA (7 mL) was added NaH (219 mg of 60% w/w, 5.476 mmol). The reaction was stirred at 0° C. for 45 min. benzenesulfonyl chloride (696 μL, 5.454 mmol) was added and the reaction was allowed to warm up to rt and stirred at rt for 22 h. The reaction was quenched with MeOH and the solvent was evaporated under reduced pressure. EtOAc was added to the reaction and washed with water (×1). The aqueous layer was extracted with EtOAc (×3). The organic layer was dried over Na₂SO₄, filtered and concentrated. The crude product was purified on 80 g of silica gel utilizing a gradient of 0-15% ethyl acetate in DCM to yield N-(4-chloro-5,6,7,8-tetrahydroquinazolin-2-yl)benzenesulfonamide (141 mg, 8%) as a yellow solid. ESI-MS m/z calc. 323.04953, found 324.1 (M+1)⁺; Retention time: 1.5 minutes; LC method A.

### Step 2: N-(4-phenoxy-5,6,7,8-tetrahydroquinazolin-2-yl)benzenesulfonamide

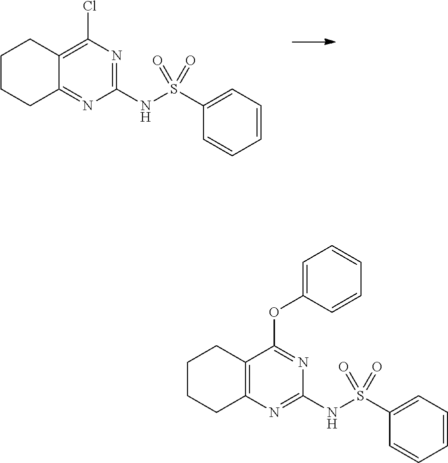 [SMILES: O=S(=O)(Nc1nc(Cl)c2c(n1)CCCC2)c1ccccc1 ; O=S(=O)(Nc1nc2c(c(Oc3ccccc3)n1)CCCC2)c1ccccc1]

To N-(4-chloro-5,6,7,8-tetrahydroquinazolin-2-yl)benzenesulfonamide (141 mg, 0.4355 mmol), sodium phenoxide (177 mg, 1.525 mmol) and DMF (2.5 mL) were added and the reaction was stirred at 110° C. for 3.5 h. The crude product was filtered and purified using a reverse phase HPLC C₁₈ column and a dual gradient run from 1-99% mobile phase B over 30 minutes (Mobile phase A=H₂O (5 mM HCl). Mobile phase B=CH₃CN) to yield N-(4-phenoxy-5,6,7,8-tetrahydroquinazolin-2-yl)benzenesulfonamide (30.3 mg, 18%) as a white solid. ¹H NMR (400 MHz, Chloroform-d) δ 7.55-7.46 (m, 4H), 7.45-7.38 (m, 1H), 7.37-7.32 (m, 1H), 7.25-7.18 (m, 2H), 7.18-7.11 (m, 2H), 2.74 (t, J=5.8 Hz, 2H), 2.62 (t, J=5.8 Hz, 2H), 1.93-1.71 (m, 4H). ESI-MS m/z calc. 381.11472, found 382.1 (M+1)⁺; Retention time: 2.11 minutes (LC method I).

### Compound 5

### N-[8,8-Dimethyl-4-(2-methylphenoxy)-6,7-dihydro-5H-quinazolin-2-yl]-1-methyl-pyrazole-4-sulfonamide

### Step 1: 8,8-Dimethyl-4-(2-methylphenoxy)-6,7-dihydro-5H-quinazolin-2-amine

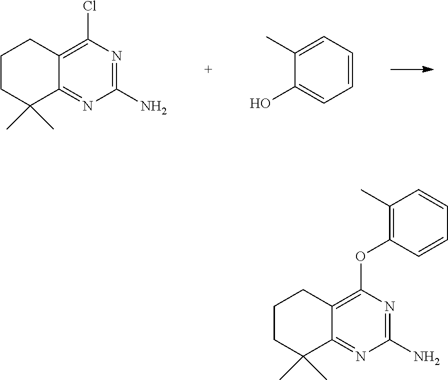

To 4-chloro-8,8-dimethyl-6,7-dihydro-5H-quinazolin-2-amine (150 mg, 0.7086 mmol) and potassium carbonate (308 mg, 2.229 mmol) was added DMF (1.7 mL) followed by o-cresol (139 μL, 0.7108 mmol). The mixture was heated at 110° C. for 18 h. The reaction was heated at 130° C. for 72 h. EtOAc and water were added to the reaction. The two layers were separated after shaking. The aqueous layer was extracted with EtOAc (×1). The organic layer was dried over Na₂SO₄, filtered and concentrated. The crude product was purified on 40 g of silica gel utilizing a gradient of 0-60% ethyl acetate in hexane to yield 8,8-dimethyl-4-(2-methylphenoxy)-6,7-dihydro-5H-quinazolin-2-amine (130.7 mg, 65%) as a yellow solid. ESI-MS m/z calc. 283.16846, found 284.2 (M+1)⁺; Retention time: 1.29 minutes; LC method A.

### Step 2: N-[8,8-Dimethyl-4-(2-methylphenoxy)-6,7-dihydro-5H-quinazolin-2-yl]-1-methyl-pyrazole-4-sulfonamide

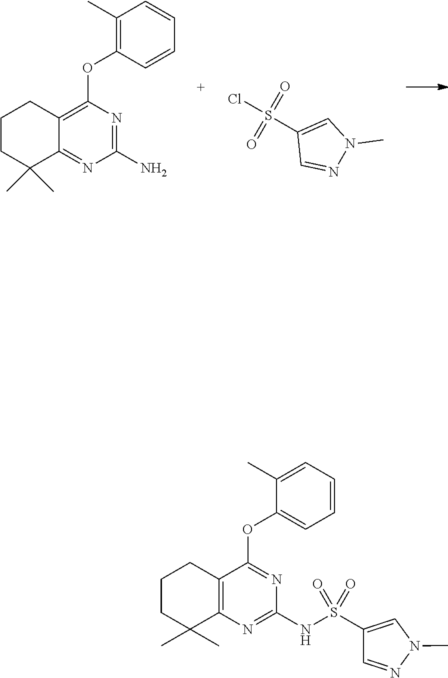

To 8,8-dimethyl-4-(2-methylphenoxy)-6,7-dihydro-5H-quinazolin-2-amine (75 mg, 0.2647 mmol), 1-methylpyrazole-4-sulfonyl chloride (192.4 mg, 1.065 mmol) and pyridine (4 mL) were added and the reaction was stirred at 110° C. for 23 h. Water and EtOAc were added to the reaction and the two layers were separated. The organic layer was washed with water (×2), dried over Na₂SO₄, filtered and the solvent was evaporated under reduced pressure. It was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B=CH₃CN). The product was impure. It was dissolved in DMSO, filtered and purified using a reverse phase HPLC C₁₈ column and a dual gradient run from 1-99% mobile phase B over 30 minutes (Mobile phase A=H₂O (5 mM HCl). Mobile phase B=CH₃CN) to yield N-[8,8-dimethyl-4-(2-methylphenoxy)-6,7-dihydro-5H-quinazolin-2-yl]-1-methyl-pyrazole-4-sulfonamide (68.4 mg, 60%) as a white solid. ¹H NMR (400 MHz, DMSO-d₆) δ 11.07 (s, 1H), 7.51-7.30 (m, 3H), 7.30-7.22 (m, 1H), 7.15 (dd, J=7.8, 1.4 Hz, 2H), 3.73 (s, 3H), 2.70-2.57 (m, 2H), 2.10 (s, 3H), 1.79-1.72 (m, 2H), 1.72-1.59 (m, 2H), 1.22 (s, 6H). ESI-MS m/z calc. 427.16782, found 428.2 (M+1)⁺; Retention time: 1.75 minutes; LC method A.

### Compound 6

### N-(4-phenoxy-6,7-dihydro-5H-cyclopenta[d]pyrimidin-2-yl)benzenesulfonamide

### Step 1: N-(4-chloro-6,7-dihydro-5H-cyclopenta[d]pyrimidin-2-yl)benzenesulfonamide

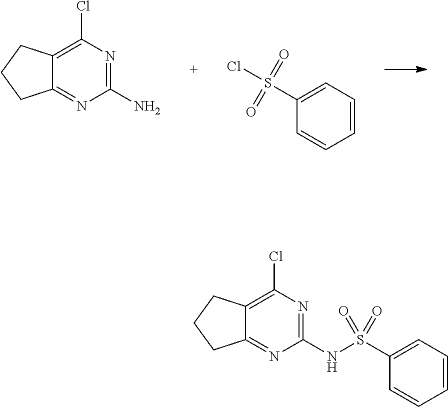 [SMILES: Nc1nc(Cl)c2c(n1)CCC2 ; O=S(=O)(Cl)c1ccccc1 ; O=S(=O)(Nc1nc(Cl)c2c(n1)CCC2)c1ccccc1]

To 4-chloro-6,7-dihydro-5H-cyclopenta[d]pyrimidin-2-amine (1 g, 5.896 mmol) in DMF (7.500 mL) was added NaH (236 mg of 60% w/w, 5.901 mmol) at 0° C. The reaction was stirred at 0° C. for 60 min. Benzenesulfonyl chloride (753 μL, 5.900 mmol) was added and the reaction was allowed to warm up to rt and stirred at rt for 22 h. The reaction was quenched with MeOH and the solvent was evaporated under reduced pressure. EtOAc was added to the reaction and washed with water (×1). The aqueous layer was extracted with EtOAc (×3). The organic layer was dried over Na₂SO₄, filtered and concentrated. The crude product was purified on 80 g of silica gel utilizing a gradient of 0-15% ethyl acetate in dichloromethane to yield N-(4-chloro-6,7-dihydro-5H-cyclopenta[d]pyrimidin-2-yl)benzenesulfonamide (167 mg, 9%) as a brown viscous solid. ESI-MS m/z calc. 309.03387, found 310.1 (M+1)⁺; Retention time: 1.4 minutes; LC method A.

### Step 2: N-(4-phenoxy-6,7-dihydro-5H-cyclopenta[d]pyrimidin-2-yl)benzenesulfonamide

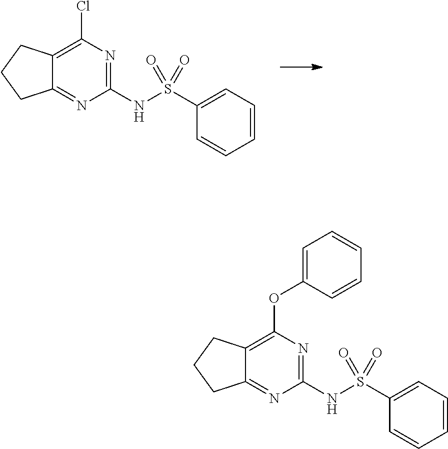 [SMILES: O=S(=O)(Nc1nc(Cl)c2c(n1)CCC2)c1ccccc1 ; O=S(=O)(Nc1nc2c(c(Oc3ccccc3)n1)CCC2)c1ccccc1]

To N-(4-chloro-6,7-dihydro-5H-cyclopenta[d]pyrimidin-2-yl)benzenesulfonamide (167 mg, 0.5391 mmol), sodium phenoxide (219 mg, 1.886 mmol) and DMF (3 mL) were added and the reaction was stirred at 110° C. for 2 h. The crude product was filtered and purified using a reverse phase HPLC C₁₈ column and a dual gradient run from 1-99% mobile phase B over 30 minutes (Mobile phase A=H₂O (5 mM HCl). Mobile phase B=CH₃CN) to yield N-(4-phenoxy-6,7-dihydro-5H-cyclopenta[d]pyrimidin-2-yl)benzenesulfonamide (58.7 mg, 30%) as a cream-colored solid. ¹H NMR (400 MHz, DMSO-d₆) δ 11.55 (s, 1H), 7.61-7.44 (m, 3H), 7.44-7.23 (m, 5H), 7.22-7.09 (m, 2H), 2.91-2.71 (m, 4H), 2.15-1.96 (m, 2H). ESI-MS m/z calc. 367.09906, found 368.2 (M+1)⁺; Retention time: 1.52 minutes; LC method A.

### Compound 7

The compound in the following Table 3 was prepared in a manner analogous to that described above using commercially available reagents and intermediates described herein.

TABLE 3
Characterization of Compound 7

| | | LCMS | | | | |
|---|---|---|---|---|---|---|
| Cmpd | | Rt | Calc. | | LCMS | |
| No. | Structure | (min) | mass | M + 1 | Met. | NMR |
| 7 | 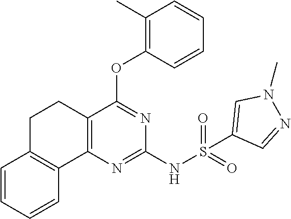 [SMILES: Cc1ccccc1Oc1nc(NS(=O)(=O)c2cnn(C)c2)nc2c1CCc1ccccc1-2] | 1.9 | 447.137 | 448.2 | A | ¹H NMR (400 MHz, DMSO- d₆) δ 11.37 (s, 1H), 8.09 (dd, J = 7.6, 1.6 Hz, 1H), 7.54 (s, 1H), 7.48- 7.38 (m, 3H), 7.38-7.32 (m, 2H), 7.30- 7.24 (m, 2H), 7.21 (dd, J = 7.9, 1.4 Hz, |
| | | | | | | 1H), 3.75 (s, |
| | | | | | | 3H), 3.09- |
| | | | | | | 2.78 (m, 4H), |
| | | | | | | 2.13 (s, 3H) |

### Compound 8

### N-[4-(2,6-dimethylphenyl)-7-methyl-7-phenyl-5,6-dihydrocyclopenta[d]pyrimidin-2-yl]benzenesulfonamide

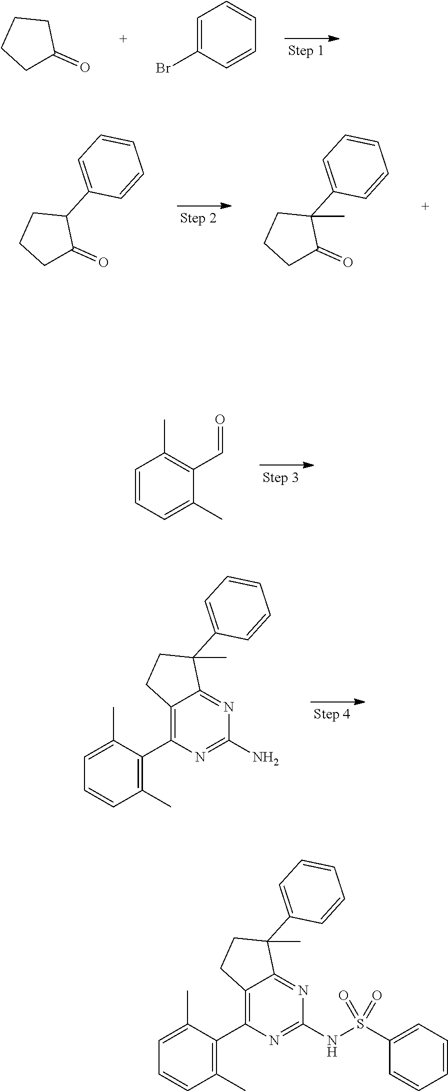

### Step 1: 2-Phenylcyclopentanone

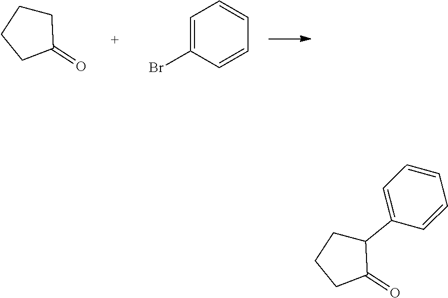 [SMILES: O=C1CCCC1 ; Brc1ccccc1 ; O=C1CCCC1c1ccccc1]

To a 100 mL round-bottomed flask equipped with a stir bar, bromobenzene (3.150 g, 20.06 mmol), cyclopentanone (1.33 mL, 15.04 mmol), pyrrolidine (0.39 mL, 4.672 mmol), tert-octyl amine (0.75 mL, 4.671 mmol), NaOAc (1.430 g, 17.43 mmol), P(o-tol)3 (0.320 g, 1.051 mmol), Pd(Oac)₂ (0.105 g, 0.4677 mmol), and dioxane (50 mL) were added, in this order. This mixture was sparged with nitrogen gas for 15 min. A reflux condenser was installed on top of the round-bottomed flask, and this mixture was stirred at 110° C. (reflux) for 40 h. The mixture was then cooled to room temperature, partially purified by a silica gel plug (20 g of silica, elution with 300 mL of 1:1 ethyl acetate:hexanes), and evaporated in vacuo to give a dark brown oil. Purification by silica gel chromatography (120 g of silica, 0 to 30% gradient of ethyl acetate/hexanes) gave a dark-orange viscous liquid that solidified in the fridge: 2-Phenylcyclopentanone (1.5107 g, 63%). ¹H NMR (400 MHz, dimethylsulfoxide-d6) δ 7.34-7.28 (m, 2H), 7.26-7.21 (m, 1H), 7.21-7.16 (m, 2H), 3.48-3.34 (m, 1H), 2.42-2.35 (m, 1H), 2.35-2.27 (m, 2H), 2.09-1.96 (m, 2H), 1.94-1.78 (m, 1H). ESI-MS m/z calc. 160.08882, found 161.1 (M+1)⁺; Retention time: 1.23 minutes; LC method A.

### Step 2: 2-Methyl-2-phenyl-cyclopentanone

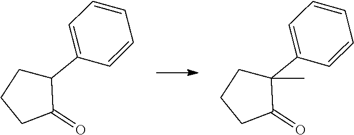

In a 100 mL round-bottomed flask, 2-phenylcyclopentanone (0.7664 g, 4.784 mmol) was dissolved in dimethylformamide (16 mL) and this solution was cooled to 0° C.; 60% NaH (0.2511 g, 6.278 mmol) was added, and this slurry was stirred at 0° C. for 10 min. Then, methyl iodide (0.400 mL, 6.425 mmol) was added, and the reaction mixture was stirred at 0° C. for 30 min. The reaction mixture was quenched with 1 N HCl (20 mL) and extracted with ethyl acetate (3×40 mL). The combined organic extracts were washed with water (50 mL) and saturated aqueous sodium chloride solution (50 mL), then dried over sodium sulfate, filtered, and evaporated in vacuo. This crude product was purified by silica gel chromatography (80 g of silica, 0 to 10% gradient of ethyl acetate/hexanes) to give 2-methyl-2-phenyl-cyclopentanone (444.8 mg, 53%) ESI-MS m/z calc. 174.10446, found 175.2 (M+1)⁺; Retention time: 1.42 minutes; LC method A.

### Step 3: 4-(2,6-Dimethylphenyl)-7-methyl-7-phenyl-5,6-dihydrocyclopenta[d]pyrimidin-2-amine

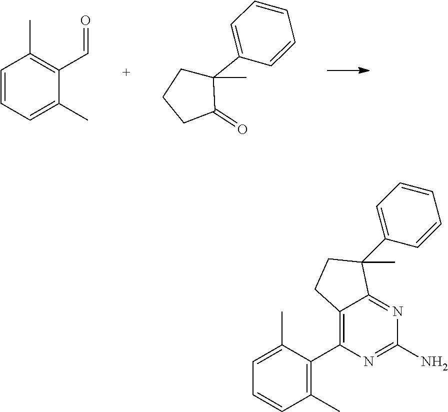

To a 20 mL vial with a pressure-relief cap, 2-methyl-2-phenyl-cyclopentanone (306.5 mg, 1.759 mmol), 2,6-dimethylbenzaldehyde (219.5 mg, 1.636 mmol), potassium carbonate (350.6 mg, 2.537 mmol) and ethanol (8.0 mL) were added, and this slurry was stirred at 80° C. for 6 h. The reaction mixture was then cooled to room temperature, quenched with 1 N HCl (20 mL), then extracted with ethyl acetate (3×20 mL). The combined organic extracts was washed with water (40 mL) and saturated aqueous sodium chloride solution (40 mL), then dried over sodium sulfate, filtered, and evaporated in vacuo. The crude product, (5E)-5-[(2,6-dimethylphenyl)methylene]-2-methyl-2-phenyl-cyclopentanone (474.2 mg, 100%) ESI-MS m/z calc. 290.16705, found 291.4 (M+1)⁺; Retention time: 2.19 minutes; LC method A.

In a 20 mL vial, the crude product from above, guanidine (carbonic acid (0.5)) (445.8 mg, 4.949 mmol) and potassium carbonate (449.8 mg, 3.255 mmol) were mixed together with N-methylpyrrolidinone (8.0 mL), and stirred at 170° C. for 62 h. This mixture was cooled to room temperature, filtered, and purified by reverse phase HPLC (1-99% acetonitrile in water using HCl as a modifier) to give recovered 5-[(2,6-dimethylphenyl)methylene]-2-methyl-2-phenyl-cyclopentanone (271.4 mg, 0.9346 mmol, 57% recovered), as well as 4-(2,6-dimethylphenyl)-7-methyl-7-phenyl-5,6-dihydrocyclopenta[d]pyrimidin-2-amine (10.2 mg, 2%) ESI-MS m/z calc. 329.1892, found 330.3 (M+1)⁺; Retention time: 1.47 minutes; LC method A.

### Step 4: N-[4-(2,6-dimethylphenyl)-7-methyl-7-phenyl-5,6-dihydrocyclopenta[d]pyrimidin-2-yl]benzenesulfonamide

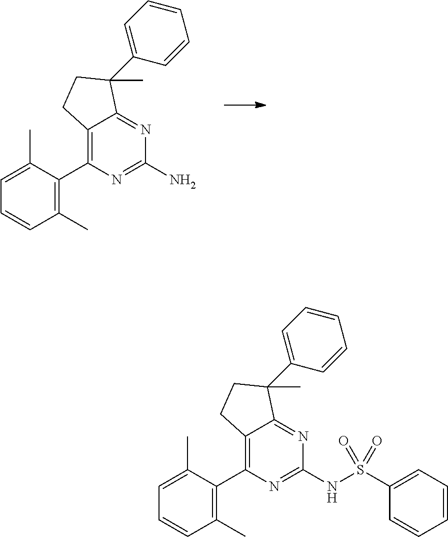 [SMILES: Cc1cccc(C)c1-c1nc(N)nc2c1CCC2(C)c1ccccc1 ; Cc1cccc(C)c1-c1nc(NS(=O)(=O)c2ccccc2)nc2c1CCC2(C)c1ccccc1]

In a 3 mL vial, 4-(2,6-dimethylphenyl)-7-methyl-7-phenyl-5,6-dihydrocyclopenta[d]pyrimidin-2-amine (10.2 mg, 0.03096 mmol) was dissolved in NMP (800 μL), to which 60% NaH (3.1 mg, 0.07751 mmol) and PhSO₂Cl (30 μL, 0.24 mmol) were added. This mixture was stirred at 90° C. for 18 h. It was then cooled to room temperature, and NaOtBu (10.3 mg, 0.107 mmol) and PhSO₂Cl (30 μL, 0.24 mmol) were added. This mixture was stirred at 90° C. for 18 h, then at 190° C. for 82 h. It was then cooled to room temperature, filtered, and purified by reverse phase HPLC (1-99% acetonitrile in water using HCl as a modifier) to give recovered starting material (5.1 mg, 50%) and N-[4-(2,6-dimethylphenyl)-7-methyl-7-phenyl-5,6-dihydrocyclopenta[d]pyrimidin-2-yl]benzenesulfonamide (0.1 mg, 1%) ESI-MS m/z calc. 469.1824, found 470.4 (M+1)⁺; Retention time: 2.11 minutes; LC method A.

### Compound 9

### N-(4-phenyl-4H-3,1-benzothiazin-2-yl)benzenesulfonamide

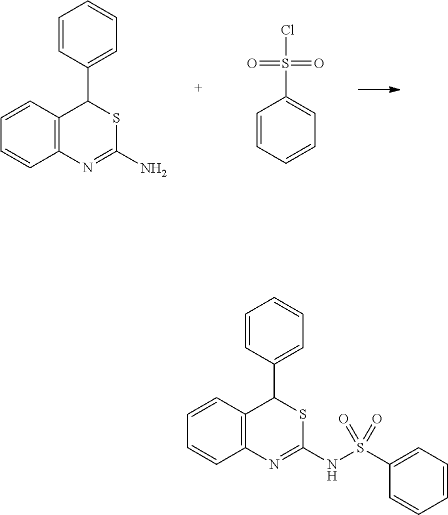 [SMILES: NC1=Nc2ccccc2C(c2ccccc2)S1 ; O=S(=O)(Cl)c1ccccc1 ; O=S(=O)(NC1=Nc2ccccc2C(c2ccccc2)S1)c1ccccc1]

To a mixture of 4-phenyl-4H-3,1-benzothiazin-2-amine (15 mg, 0.06242 mmol) was added PhSO₂Cl (approximately 16.54 mg, 11.95 μL, 0.09363 mmol) and the reaction mixture stirred at 40° C. for 4 hours. The reaction mixture was diluted with MeOH, filtered and purification by HPLC (1-99% ACN in water (HCl modifier)) gave N-(4-phenyl-4H-3,1-benzothiazin-2-yl)benzenesulfonamide (8.92 mg, 37%). ESI-MS m/z calc. 380.0653, found 381.1 (M+1)⁺; Retention time: 1.58 minutes; LC method A.

### Preparation of Ethyl 2-(benzenesulfonamido)-4,6-diphenyl-6H-1,3-thiazine-5-carboxylate

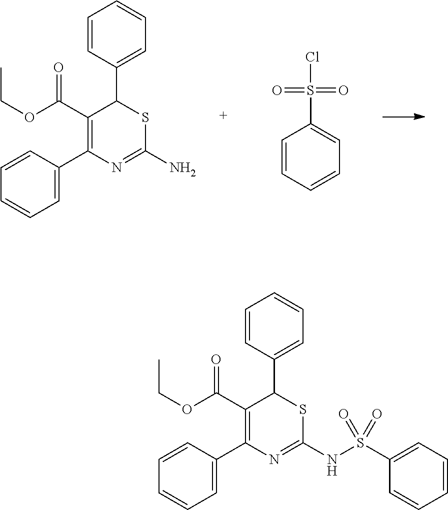 [SMILES: CCOC(=O)C1=C(c2ccccc2)N=C(N)SC1c1ccccc1 ; O=S(=O)(Cl)c1ccccc1 ; CCOC(=O)C1=C(c2ccccc2)N=C(NS(=O)(=O)c2ccccc2)SC1c1ccccc1]

To a mixture of ethyl 2-amino-4,6-diphenyl-6H-1,3-thiazine-5-carboxylate (20 mg, 0.05910 mmol) and DABCO (20 mg) was added PhSO₂Cl (11 μL, 0.08865 mmol) and the reaction mixture stirred at 40° C. for 4 hours. The reaction mixture was diluted with MeOH, filtered and purification by HPLC (1-99% ACN in water (HCl modifier)) gave ethyl 2-(benzenesulfonamido)-4,6-diphenyl-6H-1,3-thiazine-5-carboxylate (hydrochloride salt) (18 mg, 59%). ESI-MS m/z calc. 478.1021, found 479.2 (M+1)⁺; Retention time: 1.77 minutes; LC method A.

### Preparation of N-(4,6-Diphenyl-4H-1,3-thiazin-2-yl)benzenesulfonamide

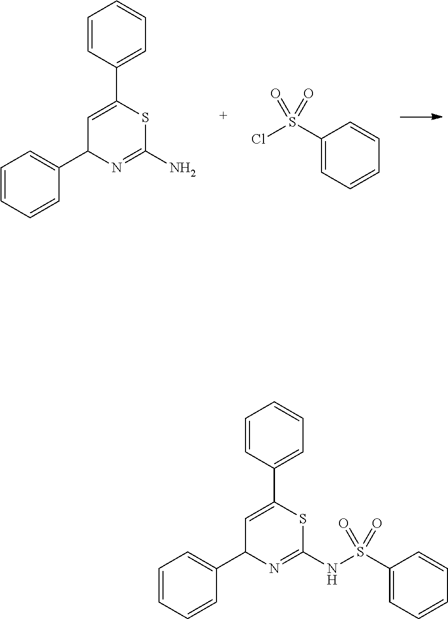 [SMILES: NC1=NC(c2ccccc2)C=C(c2ccccc2)S1 ; O=S(=O)(Cl)c1ccccc1 ; O=S(=O)(NC1=NC(c2ccccc2)C=C(c2ccccc2)S1)c1ccccc1]

To a 10 mL vial equipped with a magnetic stir bar, 4,6-diphenyl-4H-1,3-thiazin-2-amine (16.9 mg, 0.06345 mmol), acetonitrile (500 μL), DABCO (20.0 mg, 0.1783 mmol), and benzenesulfonyl chloride (20 μL, 0.1567 mmol) were added, in this order. The vial was capped and stirred at room temperature for 20 min, upon which the reaction mixture was diluted with 1:1 methanol:dimethylsulfoxide (500 μL), filtered, and purified by reverse phase HPLC (1-99% acetonitrile in water using HCl as a modifier) to give N-(4,6-diphenyl-4H-1,3-thiazin-2-yl)benzenesulfonamide (2.1 mg, 7%). ESI-MS m/z calc. 406.08096, found 407.1 (M+1)⁺; Retention time: 1.77 minutes; LC method A.

### Compound 10

### N-[3-(2-tert-butylphenoxy)quinoxalin-2-yl]benzenesulfonamide

### Step 1: N-(3-chloroquinoxalin-2-yl)benzenesulfonamide

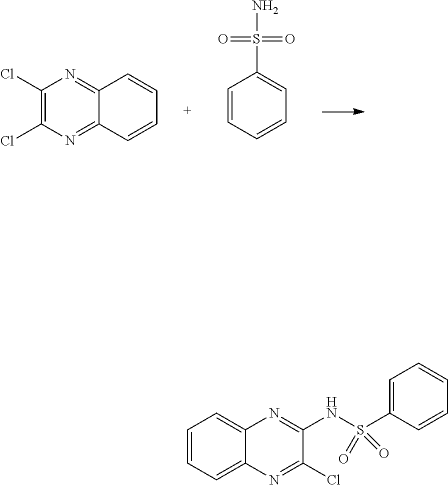 [SMILES: Clc1nc2ccccc2nc1Cl ; NS(=O)(=O)c1ccccc1 ; O=S(=O)(Nc1nc2ccccc2nc1Cl)c1ccccc1]

A mixture of benzenesulfonamide (approximately 789.7 mg, 5.024 mmol), 2,3-dichloroquinoxaline (1 g, 5.024 mmol) and potassium carbonate (approximately 833.2 mg, 6.029 mmol) in DMSO (10 mL) was heated to 150° C. for 1 h. After cooling down, water was added to the reaction mixture then to this was added slowly 2N HCl (aq) to precipitate a product which was filtered and dried to give N-(3-chloroquinoxalin-2-yl)benzenesulfonamide (1.2 g, 75%). ESI-MS m/z calc. 319.01822, found 320.06 (M+1)⁺; Retention time: 0.59 minutes; LC method D.

### Step 2: N-[3-(2-tert-butylphenoxy)quinoxalin-2-yl]benzenesulfonamide

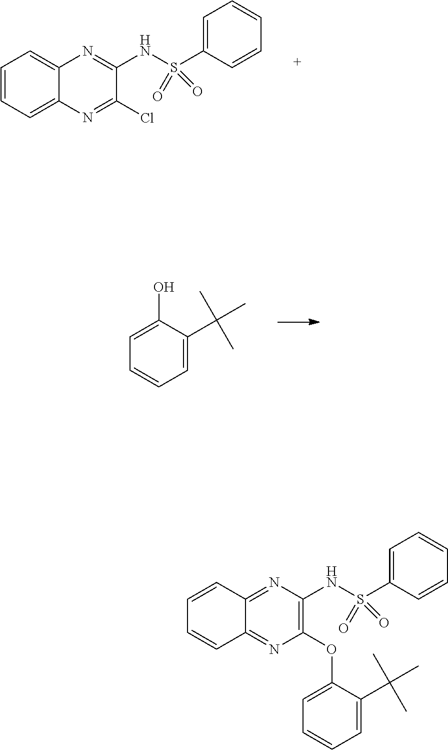 [SMILES: O=S(=O)(Nc1nc2ccccc2nc1Cl)c1ccccc1 ; CC(C)(C)c1ccccc1O ; CC(C)(C)c1ccccc1Oc1nc2ccccc2nc1NS(=O)(=O)c1ccccc1]

To a solution of N-(3-chloroquinoxalin-2-yl)benzenesulfonamide (25 mg, 0.07818 mmol) in DMSO (300 μL) was added cesium fluoride (approximately 23.76 mg, 5.774 μL, 0.1564 mmol) and the reaction was stirred overnight. This solution was added to a mixture of 2-tert-butylphenol (approximately 23.49 mg, 0.1564 mmol) and Cs₂CO₃ (approximately 76.40 mg, 0.2345 mmol) in DMSO (1 mL) in a microwave vial and the mixture was heated in a microwave for 30 min at 125° C. It was filtered and was purified by reverse phase HPLC to give N-[3-(2-tert-butylphenoxy)quinoxalin-2-yl]benzenesulfonamide (4.7 mg, 14%). ESI-MS m/z calc. 433.14603, found 434.29 (M+1)⁺; Retention time: 2.13 minutes; LC method A.

### Compound 11

### N-[3-(2,6-Dimethylphenoxy)quinoxalin-2-yl]benzenesulfonamide

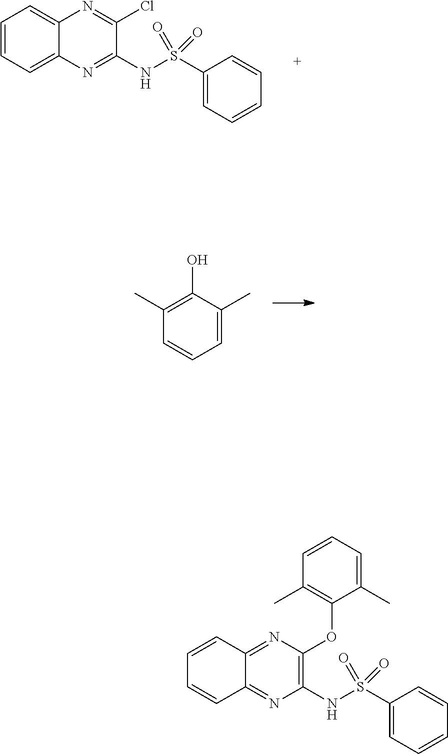

To a solution of N-(3-chloroquinoxalin-2-yl)benzenesulfonamide (25 mg, 0.07818 mmol) in DMSO (300 μL) was added cesium fluoride (approximately 23.76 mg, 5.774 μL, 0.1564 mmol) and the reaction was stirred overnight. This solution was added to a mixture of 2,6-dimethylphenol (approximately 19.11 mg, 0.1564 mmol) and Cs₂CO₃ (approximately 76.40 mg, 0.2345 mmol) in DMSO (1 mL) in microwave vial and the mixture was heated in a microwave for 30 min at 125° C. It was filtered and purified by reverse phase HPLC to give N-[3-(2,6-dimethylphenoxy)quinoxalin-2-yl]benzenesulfonamide (4.3 mg, 14%). ESI-MS m/z calc. 405.11472, found 406.27 (M+1)⁺; Retention time: 1.97 minutes; LC method A.

### Compound 12

### N-[3-(1-piperidyl)quinoxalin-2-yl]benzenesulfonamide

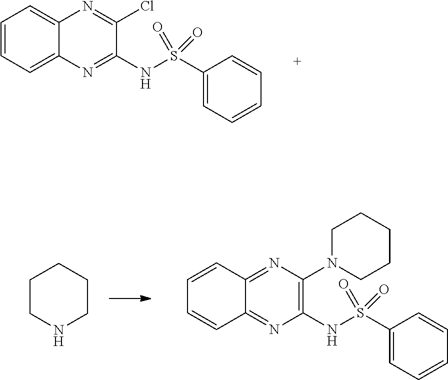 [SMILES: O=S(=O)(Nc1nc2ccccc2nc1Cl)c1ccccc1 ; C1CCNCC1 ; O=S(=O)(Nc1nc2ccccc2nc1N1CCCCC1)c1ccccc1]

To a solution of N-(3-chloroquinoxalin-2-yl)benzenesulfonamide (25 mg, 0.07818 mmol) in DMSO (500 μL) was added piperidine (approximately 19.97 mg, 23.19 μL, 0.2345 mmol) and K₂CO₃ (approximately 54.02 mg, 0.3909 mmol) and the vial was sealed and heated at 110° C. overnight. It was filtered and purified by reverse phase HPLC using 1-99% CH₃CN in water (HCl modifier) to afford N-[3-(1-piperidyl)quinoxalin-2-yl]benzenesulfonamide (14.6 mg, 51%). ESI-MS m/z calc. 368.1307, found 369.44 (M+1)⁺; Retention time: 1.41 minutes (LC method A).

### Compounds 13-15

The compounds in the following Table 4 were prepared in a manner analogous to that described above using commercially available reagents and intermediates described herein.

TABLE 4
Characterization of Compounds 13-15

| Cmpd | | LCMS | | | |
|---|---|---|---|---|---|
| No. | Structure | Rt (min) | Calc. mass | M + 1 | LCMS Met. |
| 13 | 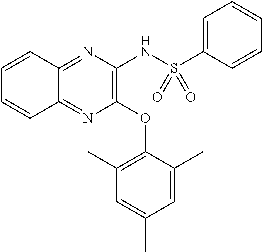 [SMILES: Cc1cc(C)c(Oc2nc3ccccc3nc2NS(=O)(=O)c2ccccc2)c(C)c1] | 2.09 | 419.13 | 420.32 | A |
| 14 | 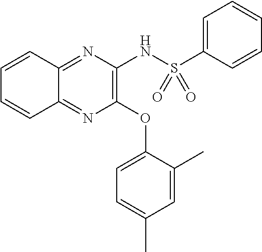 [SMILES: Cc1ccc(Oc2nc3ccccc3nc2NS(=O)(=O)c2ccccc2)c(C)c1] | 2.02 | 405.115 | 406.27 | A |
| 15 | 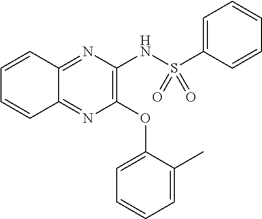 [SMILES: Cc1ccccc1Oc1nc2ccccc2nc1NS(=O)(=O)c1ccccc1] | 1.89 | 391.099 | 392.27 | A |

### Preparation of N-(2-phenoxy-6-phenylpyrimidin-4-yl)benzenesulfonamide and N-(6 phenoxy-2-phenyl-pyrimidin-4-yl)benzenesulfonamide

### Step 1: N-(2,6-Dichloropyrimidin-4-yl)benzenesulfonamide

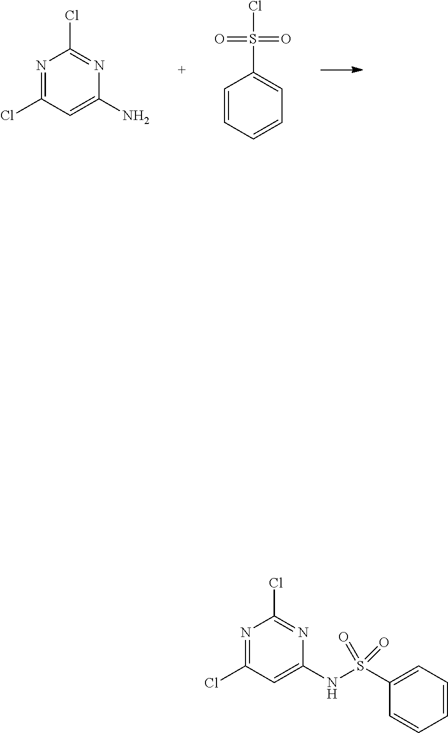 [SMILES: Nc1cc(Cl)nc(Cl)n1 ; O=S(=O)(Cl)c1ccccc1 ; O=S(=O)(Nc1cc(Cl)nc(Cl)n1)c1ccccc1]

To a solution of 2,6-dichloropyrimidin-4-amine (approximately 5.000 g, 30.49 mmol) in DMIF (64.63 mL) was added sodium hydride (approximately 1.585 g of 60% w/w, 39.64 mmol) at 0° C. and the reaction was stirred for 10 min at 0° C. To this mixture was added dropwise benzenesulfonyl chloride (approximately 6.463 g, 4.670 mL, 36.59 mmol) and the reaction was stirred at 0° C. for 10 min. The reaction mixture was slowly poured into ice water. It was acidified with 1N HCl, extracted with ethyl acetate (2×30 mL). The organic layer was separated, dried over Na₂SO₄, concentrated and the residue was purified by silica gel chromatography using a gradient of ethyl acetate/hexane. The product eluted around ˜25% ethyl acetate to give N-(2,6-dichloropyrimidin-4-yl)benzenesulfonamide (3.8 g, 340%) as a white solid. ¹H NMR (400 MHz, DMSO) δ 8.00 (d, J=7.6 Hz, 2H), 7.73 (t, J=7.3 Hz, 1H), 7.65 (t, J=7.6 Hz, 2H), 6.99 (s, 1H). ESI-MS m/z calc. 302.9636, found 304.0 (M+1)⁺; Retention time: 1.38 minutes (LC method A).

### Step 2: N-(6-chloro-2-phenyl-pyrimidin-4-yl)benzenesulfonamide and N-(2-chloro-6-phenyl-pyrimidin-4-yl)benzenesulfonamide

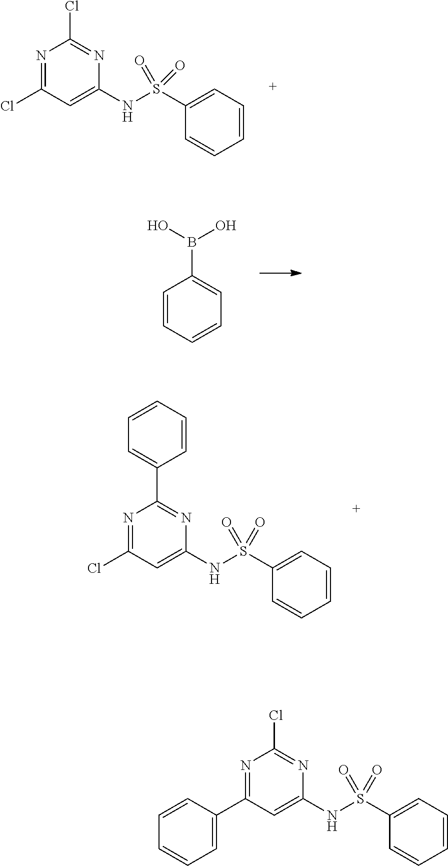 [SMILES: O=S(=O)(Nc1cc(Cl)nc(Cl)n1)c1ccccc1 ; OB(O)c1ccccc1 ; O=S(=O)(Nc1cc(Cl)nc(-c2ccccc2)n1)c1ccccc1 ; O=S(=O)(Nc1cc(-c2ccccc2)nc(Cl)n1)c1ccccc1]

To a mixture of N-(2,6-dichloropyrimidin-4-yl)benzenesulfonamide (500 mg, 1.627 mmol), phenylboronic acid (approximately 238.0 mg, 1.952 mmol) in DMF (7 mL) was added sodium carbonate (approximately 3.254 mL of 2 M, 6.508 mmol), Pd(dppf)Cl₂ (approximately 119.0 mg, 0.1627 mmol). The mixture was thoroughly flushed with nitrogen and heated at 90° C. for 1 h. The reaction mixture was diluted with ethyl acetate, extracted with 1N HCl. The organic layer was dried over Na₂SO₄, concentrated under reduced pressure. The crude was purified on reverse phase HPLC (HCl modifier, 35-70% ACN-H₂O) to give:

Peak 1: N-(6-chloro-2-phenyl-pyrimidin-4-yl)benzenesulfonamide (24.8 mg, 4%). ¹H NMR (400 MHz, DMSO-d₆) δ 12.41 (s, 1H), 8.09-8.02 (m, 2H), 8.00-7.93 (m, 2H), 7.76-7.69 (m, 1H), 7.69-7.63 (m, 2H), 7.60-7.52 (m, 3H), 7.38 (s, 1H). ESI-MS m/z calc. 345.03387, found 346.1 (M+1)⁺; Retention time: 1.68 minutes (LC method A).

Peak 2: N-(2-chloro-6-phenyl-pyrimidin-4-yl)benzenesulfonamide (96.3 mg, 16%). ¹H NMR (400 MHz, DMSO-d₆) δ 12.27 (s, 1H), 8.13 (d, J=6.6 Hz, 2H), 8.06 (d, J=7.9 Hz, 2H), 7.71-7.59 (m, 3H), 7.59-7.48 (m, 3H), 6.91 (s, 1H). ESI-MS m/z calc. 345.03387, found 346.1 (M+1)⁺; Retention time: 1.74 minutes (LC method A).

### Step 3: N-(2-phenoxy-6-phenylpyrimidin-4-yl)benzenesulfonamide and N-(6-phenoxy-2-phenyl-pyrimidin-4-yl)benzenesulfonamide

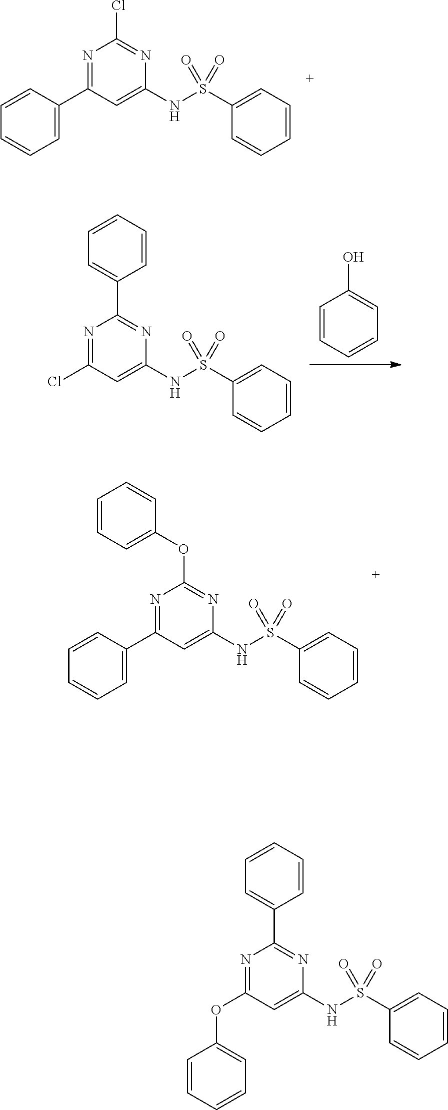 [SMILES: O=S(=O)(Nc1cc(-c2ccccc2)nc(Cl)n1)c1ccccc1 ; O=S(=O)(Nc1cc(Cl)nc(-c2ccccc2)n1)c1ccccc1 ; Oc1ccccc1 ; O=S(=O)(Nc1cc(-c2ccccc2)nc(Oc2ccccc2)n1)c1ccccc1 ; O=S(=O)(Nc1cc(Oc2ccccc2)nc(-c2ccccc2)n1)c1ccccc1]

A mixture of phenol (approximately 8.165 mg, 7.703 μL, 0.08676 mmol), sodium carbonate (approximately 10.41 mg, 0.1735 mmol), and either N-(2-chloro-6-phenyl-pyrimidin-4-yl)benzenesulfonamide (approximately 20.00 mg, 0.05784 mmol) or, in a separated vial, N-(6-chloro-2-phenyl-pyrimidin-4-yl)benzenesulfonamide (approximately 20.00 mg, 0.05784 mmol) in DMSO (500 μL) was heated at 105° C. for 15 h and then at 120° C. for 20 h. After cooling down, each reaction mixture was filtered and purified by reverse phase HPLC (HCl modifier, 25-75% ACN-H₂O) to give two products:

N-(2-phenoxy-6-phenylpyrimidin-4-yl)benzenesulfonamide (8.6 mg). ¹H NMR (400 MHz, DMSO) δ 11.96 (s, 1H), 7.92-7.86 (m, 2H), 7.65 (d, J=7.4 Hz, 3H), 7.57-7.47 (m, 7H), 7.34 (t, J=7.1 Hz, 1H), 7.22 (d, J=8.5 Hz, 2H), 7.11 (s, 1H). ESI-MS m/z calc. 403.09906, found 404.0 (M+1)⁺; Retention time: 1.86 minutes (LC method A).

N-(6-phenoxy-2-phenyl-pyrimidin-4-yl)benzenesulfonamide (4.4 mg, 18%). ¹H NMR (400 MHz, DMSO) δ 11.86 (s, 1H), 8.04-7.97 (m, 4H), 7.69-7.60 (m, 3H), 7.47 (dt, J=23.4, 7.2 Hz, 5H), 7.33 (t, J=7.4 Hz, 1H), 7.24 (d, J=7.7 Hz, 2H), 6.25 (s, 1H). ESI-MS m/z calc. 403.09906, found 404.0 (M+1)⁺; Retention time: 1.93 minutes (LC method A).

### Compounds 16-19

The compounds in the following Table 5 were prepared in a manner analogous to that described above using commercially available reagents and intermediates described herein.

TABLE 5
Characterization of Compounds 16-19

| | | LCMS | | | | |
|---|---|---|---|---|---|---|
| Cmpd | | Rt | Calc. | | LCMS | |
| No. | Structure | (min) | mass | M + 1 | Met. | NMR |
| 16 | 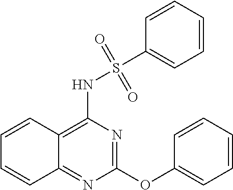 [SMILES: O=S(=O)(Nc1nc(Oc2ccccc2)nc2ccccc12)c1ccccc1] | 1.29 | 377.083 | 378.1 | A | |
| 17 | 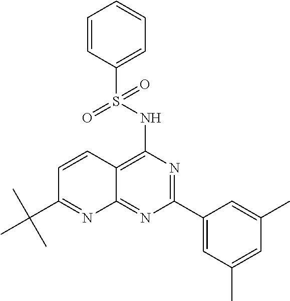 [SMILES: Cc1cc(C)cc(-c2nc(NS(=O)(=O)c3ccccc3)c3ccc(C(C)(C)C)nc3n2)c1] | 1.94 | 446.178 | 447.2 | A | ¹H NMR (400 MHz, DMSO-d₆) δ 13.73 (s, 1H), 8.59 (s, 1H), 7.91 (s, 2H), 7.77 (d, J = 8.4 Hz, 1H), 7.66-7.42 (m, 5H), 7.27 (s, 1H), 2.36 (s, 6H), 1.42 (s, 9H). |
| 18 | 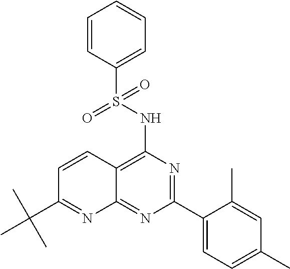 [SMILES: Cc1ccc(-c2nc(NS(=O)(=O)c3ccccc3)c3ccc(C(C)(C)C)nc3n2)c(C)c1] | 1.9 | 446.178 | 447.4 | A | ¹H NMR (400 MHz, DMSO-d₆) δ 13.89 (s, 1H), 8.53 (d, J = 8.6 Hz, 1H), 7.83 (d, J = 7.8 Hz, 2H), 7.75 (d, J = 8.5 Hz, 1H), 7.57- 7.47 (m, 1H), 7.47-7.37 (m, 2H), 7.22 (d, J = 7.7 Hz, 1H), 7.17 (s, 1H), 7.13 (d, J = 7.8 Hz, 1H), 2.37 (s, 3H), 2.29 (s, 3H), 1.39 (s, 9H). |
| 19 | 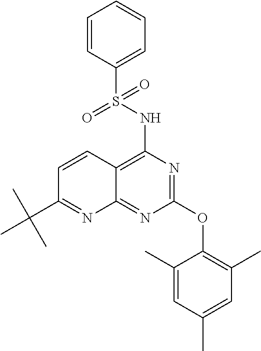 [SMILES: Cc1cc(C)c(Oc2nc(NS(=O)(=O)c3ccccc3)c3ccc(C(C)(C)C)nc3n2)c(C)c1] | 1.92 | 476.188 | 477.3 | A | |

### Compound 20

### N-(4,5-Dihydrobenzo[g][1,3]benzothiazol-2-yl) benzenesulfonamide

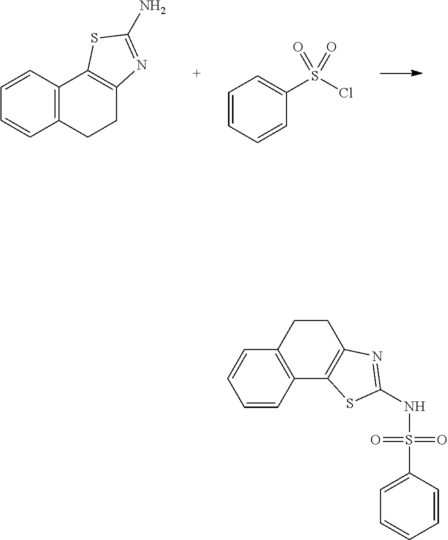 [SMILES: Nc1nc2c(s1)-c1ccccc1CC2 ; O=S(=O)(Cl)c1ccccc1 ; O=S(=O)(Nc1nc2c(s1)-c1ccccc1CC2)c1ccccc1]

Benzenesulfonyl chloride (25 mg) was added to 4,5-dihydrobenzo[g][1,3]benzothiazol-2-amine (approximately 28.62 mg, 0.1415 mmol) in pyridine (0.5 mL). The mixture was stirred at 115° C. for 1 h. The reaction mixture was filtered and purified by reverse phase HPLC using a gradient of acetonitrile and 5 mM HCl in water to give N-(4,5-dihydrobenzo[g][1,3]benzothiazol-2-yl)benzenesulfonamide (8.3 mg, 170%). ESI-MS m/z calc. 342.04968, found 343.0 (M+1)⁺; Retention time: 1.52 minutes; LC method A. ¹H NMR (400 MHz, DMSO) δ 13.25 (s, 1H), 7.85 (d, J=6.8 Hz, 2H), 7.57 (dd, J=16.4, 8.6 Hz, 4H), 7.25 (d, J=7.2 Hz, 3H), 2.96 (t, J=7.8 Hz, 2H), 2.76 (t, J=7.8 Hz, 2H).

### Compounds 21-25

The compounds in the following Tables 6 and 7 were prepared in a manner analogous to that described above using commercially available reagents and intermediates described herein.

TABLE 6
Characterization of Compounds 21-25

| Cmpd | | LCMS | | | LCMS |
|---|---|---|---|---|---|
| No. | Structure | Rt (min) | Calc. mass | M + 1 | Method |
| 21 | 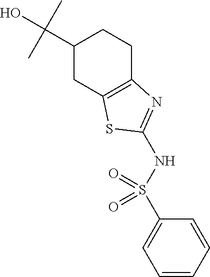 [SMILES: CC(C)(O)C1CCc2nc(NS(=O)(=O)c3ccccc3)sc2C1] | 1.18 | 352.092 | 353 | A |
| 22 | 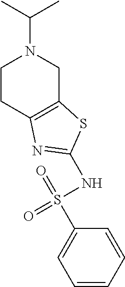 [SMILES: CC(C)N1CCc2nc(NS(=O)(=O)c3ccccc3)sc2C1] | 0.65 | 337.092 | 338 | A |
| 23 | 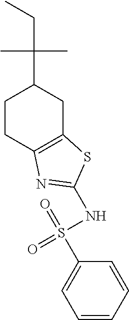 [SMILES: CCC(C)(C)C1CCc2nc(NS(=O)(=O)c3ccccc3)sc2C1] | 1.91 | 364.128 | 365 | A |
| 24 | 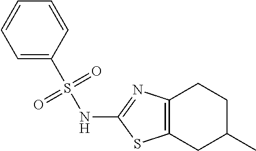 [SMILES: CC1CCc2nc(NS(=O)(=O)c3ccccc3)sc2C1] | 1.43 | 308.065 | 309 | A |
| 25 | 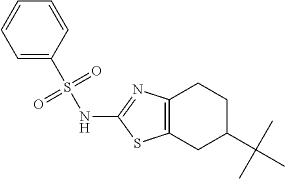 [SMILES: CC(C)(C)C1CCc2nc(NS(=O)(=O)c3ccccc3)sc2C1] | 1.79 | 350.112 | 351 | A |

TABLE 7
NMR data of Compounds 21, 23, 24

| Cmpd No. | NMR |
|---|---|
| 21 | ¹H NMR (400 MHZ, DMSO) δ 12.47 (s, 1H), 7.79 (d, J = 7.4 Hz, 2H), 7.65- |
| | 7.47 (m, 3H), 2.53 (s, 1H), 2.41 (s, 1H), 2.32 (d, J = 9.0 Hz, 1H), 2.25- |
| | 2.14 (m, 1H), 2.07 (s, 1H), 2.00 (d, J = 10.7 Hz, 1H), 1.60 (t, J = 12.0 Hz, |
| | 1H), 1.31 (dd, J = 18.1, 6.0 Hz, 1H), 1.08 (s, 6H) |
| 23 | ¹H NMR (400 MHZ, DMSO) δ 12.42 (s, 1H), 7.78 (d, J = 7.3 Hz, 2H), 7.60- |
| | 7.50 (m, 3H), 2.37 (d, J = 35.9 Hz, 3H), 2.20 (t, J = 13.7 Hz, 1H), 1.88 |
| | (d, J = 12.5 Hz, 1H), 1.52 (s, 1H), 1.27 (dd, J = 13.2, 5.7 Hz, 3H), 0.79 (dd, |
| | J = 16.3, 6.1 Hz, 9H). |
| 24 | ¹H NMR (400 MHz, DMSO) δ 12.44 (s, 1H), 7.78 (d, J = 9.4 Hz, 2H), 7.64- |
| | 7.48 (m, 3H), 2.54 (s, 1H), 2.37 (s, 2H), 2.10-1.96 (m, 1H), 1.78 (d, J = |
| | 15.5 Hz, 2H), 1.35 (t, J = 10.5 Hz, 1H), 0.99 (d, J = 6.5 Hz, 3H). |

### Compounds 26 and 27

The compounds in the following Table 8 were prepared in a manner analogous to that described above using commercially available reagents and intermediates described herein.

TABLE 8
Characterization of Compounds 26 and 27

| Cmpd | | LCMS | Calc. | | LCMS | |
|---|---|---|---|---|---|---|
| No. | Structure | Rt (min) | mass | M + 1 | Met. | NMR |
| 26 | 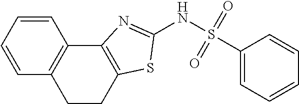 [SMILES: O=S(=O)(Nc1nc2c(s1)CCc1ccccc1-2)c1ccccc1] | 1.52 | 342.05 | 343 | A | ¹H NMR (400 MHz, DMSO) δ 13.25 (s, 1H), 7.85 (d, J =7.6 Hz, 2H), 7.59 (dt, J = 14.8, 6.4 Hz, 4H), 7.25 (d, J = 6.7 |
| | | | | | | Hz, 3H), 2.96 (t, |
| | | | | | | J = 7.9 Hz, 2H), |
| | | | | | | 2.76 (t, J = 7.9 |
| | | | | | | Hz, 2H). |
| 27 | 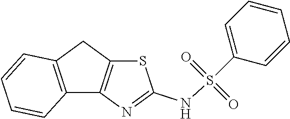 [SMILES: O=S(=O)(Nc1nc2c(s1)Cc1ccccc1-2)c1ccccc1] | 1.42 | 328.034 | 329 | A | ¹H NMR (400 MHz, DMSO) δ 13.68 (s, 1H), 7.86 (d, J = 9.4 Hz, 2H), 7.58 (dt, J = 14.9, 7.2 Hz, 4H), 7.47 (d, J = 7.5 Hz, 1H), 7.35 (t, |
| | | | | | | J = 7.5 Hz, 1H), |
| | | | | | | 7.26 (t, J = 7.5 |
| | | | | | | Hz, 1H), 3.79 (s, |
| | | | | | | 2H). |

### Compound 28

### N-[4-(3,5-Dimethylphenyl)quinazolin-2-yl]benzenesulfonamide

### Step 1: 4-(3,5-Dimethylphenyl)quinazolin-2-amine

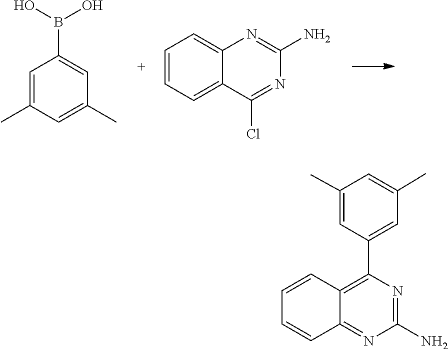

A mixture of 4-chloroquinazolin-2-amine (150 mg, 0.8352 mmol), (3,5-dimethylphenyl)boronic acid (167.1 mg, 1.114 mmol), Pd(dppf)Cl₂ (approximately 61.11 mg, 0.08352 mmol), 1,2-dimethoxyethane (1.8 mL) and potassium carbonate (approximately 835.0 μL of 2 M, 1.670 mmol) was degassed by flow of nitrogen and stirred at 130° C. for 3 h. The cooled mixture was filtered and diluted with EtOAc and washed with water (×1). The aqueous layer was extracted twice with EtOAc. The combined organic layer was dried over Na₂SO₄, filtered and concentrated in vacuo. The crude product was purified on 40 g of silica gel utilizing a gradient of 0-35% ethyl acetate in hexane to yield 4-(3,5-dimethylphenyl)quinazolin-2-amine (150 mg, 72%) as a yellow solid. ESI-MS m/z calc. 249.1266, found 250.2 (M+1)⁺; Retention time: 0.45 minutes; LC method D.

### Step 2: N-[4-(3,5-Dimethylphenyl)quinazolin-2-yl]benzenesulfonamide

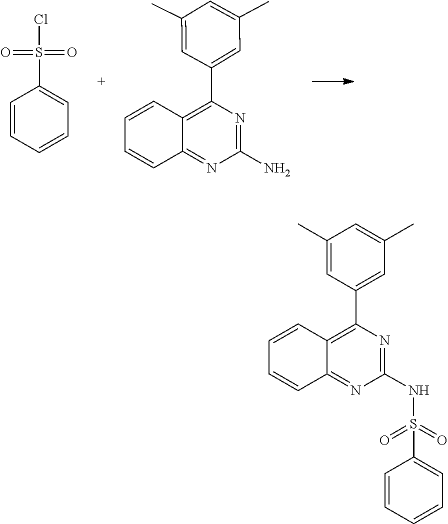 [SMILES: O=S(=O)(Cl)c1ccccc1 ; Cc1cc(C)cc(-c2nc(N)nc3ccccc23)c1 ; Cc1cc(C)cc(-c2nc(NS(=O)(=O)c3ccccc3)nc3ccccc23)c1]

To a solution of 4-(3,5-dimethylphenyl)quinazolin-2-amine (78 mg, 0.3129 mmol) in pyridine (1 mL) was added benzenesulfonyl chloride (40 μL, 0.3134 mmol) and the reaction was stirred at 200° C. for 105 min in a pressure vessel. More benzenesulfonyl chloride (40 μL, 0.3134 mmol) was added to the reaction, which was heated at 200° C. for 1 h. EtOAc was added to the reaction and it was washed with water (×3). The organic layer was dried over Na₂SO₄, filtered and concentrated. The crude product was dissolved in DMSO, filtered and purified using a reverse phase HPLC C₁₈ column and a dual gradient run from 1-99% mobile phase B over 30 minutes [(Mobile phase A=H₂O (5 mM HCl). Mobile phase B=CH₃CN)] to yield N-[4-(3,5-dimethylphenyl)quinazolin-2-yl]benzenesulfonamide (62 mg, 51%). ¹H NMR (400 MHz, DMSO-d₆) δ 11.97 (s, 1H), 8.01 (s, 2H), 7.93-7.85 (m, 2H), 7.71 (s, 1H), 7.66-7.60 (m, 1H), 7.59-7.51 (m, 2H), 7.50-7.41 (m, 1H), 7.26 (s, 1H), 7.09 (s, 2H), 2.37 (s, 6H). ESI-MS m/z calc. 389.1198, found 390.2 (M+1)⁺; Retention time: 1.73 minutes; LC method A.

### Compound 29

### N-(4,7-Diphenylpyrrolo[2,3-d]pyrimidin-2-yl)benzenesulfonamide

### Step 1: 2,4-Dichloro-7-phenyl-pyrrolo[2,3-d]pyrimidine

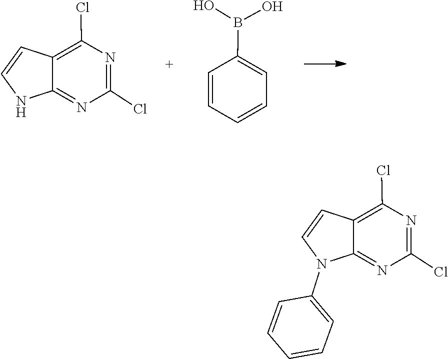 [SMILES: Clc1nc(Cl)c2cc[nH]c2n1 ; OB(O)c1ccccc1 ; Clc1nc(Cl)c2ccn(-c3ccccc3)c2n1]

To a mixture of 2,4-dichloro-7H-pyrrolo[2,3-d]pyrimidine (500 mg, 2.659 mmol), phenylboronic acid (approximately 648.4 mg, 5.318 mmol), pyridine N-oxide (approximately 278.2 mg, 2.925 mmol), TEA (approximately 1.346 g, 1.854 mL, 13.30 mmol) and pyridine (approximately 1.052 g, 1.076 mL, 13.30 mmol) was added diacetoxycopper (approximately 579.6 mg, 3.191 mmol) and the reaction mixture was stirred open to the air for 72 hours. The reaction mixture was poured into water and extracted with EtOAc (3×). Organics were combined, washed with 0.1 HCl (3×), water and brine then dried over Na₂SO₄ and evaporated to dryness. Purification by column chromatography (40 g silica; 0-30% EtOAc in hexanes) gave 2,4-dichloro-7-phenyl-pyrrolo[2,3-d]pyrimidine (622 mg, 89%) as a tan solid. ESI-MS m/z calc. 263.0017, found 264.1 (M+1)⁺; Retention time: 0.7 minutes; LC method A.

### Step 2: 2-Chloro-4,7-diphenyl-pyrrolo[2,3-d]pyrimidine

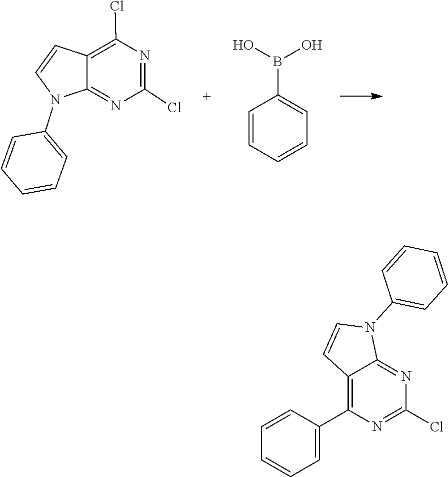 [SMILES: Clc1nc(Cl)c2ccn(-c3ccccc3)c2n1 ; OB(O)c1ccccc1 ; Clc1nc(-c2ccccc2)c2ccn(-c3ccccc3)c2n1]

A mixture of 2,4-dichloro-7-phenyl-pyrrolo[2,3-d]pyrimidine (200 mg, 0.7573 mmol), 2,4-dichloro-7-phenyl-pyrrolo[2,3-d]pyrimidine (200 mg, 0.7573 mmol), sodium carbonate (approximately 946.5 μL of 2 M, 1.893 mmol) and Pd(II)DPPF (approximately 27.70 mg, 0.03786 mmol) was stirred at 70° C. for 6 hours. The reaction mixture was diluted with water and extracted with EtOAc (3×). Organics combined and purification by column chromatography (24 g silica; 0-30% EtOAc in hexanes) gave 2-chloro-4,7-diphenyl-pyrrolo[2,3-d]pyrimidine (151 mg, 65%) as a reddish semisolid. ESI-MS m/z calc. 305.07196, found 306.2 (M+1)⁺; Retention time: 0.77 minutes; LC method D.

### Step 3: N-(4,7-Diphenylpyrrolo[2,3-d]pyrimidin-2-yl)benzenesulfonamide

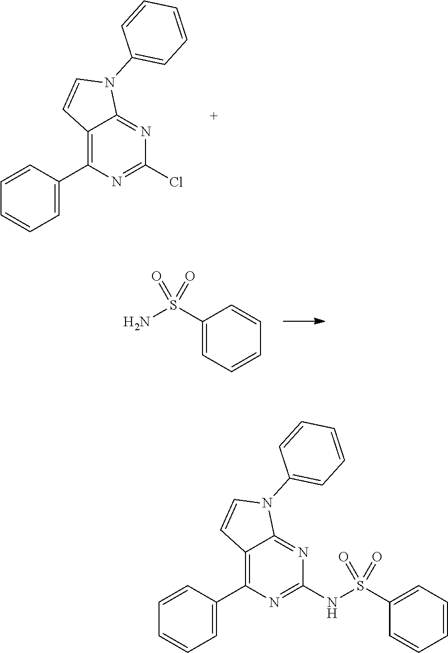 [SMILES: Clc1nc(-c2ccccc2)c2ccn(-c3ccccc3)c2n1 ; NS(=O)(=O)c1ccccc1 ; O=S(=O)(Nc1nc(-c2ccccc2)c2ccn(-c3ccccc3)c2n1)c1ccccc1]

A solution of 2-chloro-4,7-diphenyl-pyrrolo[2,3-d]pyrimidine (10 mg, 0.03271 mmol), (5-diphenylphosphanyl-9,9-dimethyl-xanthen-4-yl)-diphenyl-phosphane (approximately 5.678 mg, 0.009813 mmol) and palladium(II) diacetate (approximately 1.101 mg, 0.004906 mmol) in dioxane (0.3 mL) was stirred while nitrogen was bubbled through the solution. In a separate vial, benzenesulfonamide (approximately 15.43 mg, 0.09813 mmol) and Cs₂CO₃ (approximately 21.32 mg, 0.06542 mmol) in dioxane (0.3 mL) was stirred while nitrogen was being bubbled through the mixture. After 20 min, the Cs₂CO₃/benzenesulfonamide mixture was added to the pyrimidine, catalyst and Pd solution and the reaction mixture was stirred at 100° C. under a positive flow of nitrogen for 15 min. The reaction mixture was diluted with water and made acidic with HCl and extracted with EtOAc (3×). The organics were combined and evaporated to dryness. Purification by column chromatography (4 g silica; 0-30% EtOAc in hexanes) gave N-(4,7-diphenylpyrrolo[2,3-d]pyrimidin-2-yl)benzenesulfonamide (8.3 mg, 56%) as a white solid. ¹H NMR (400 MHz, DMSO-d₆) δ 11.67 (s, 1H), 8.08-8.00 (m, 2H), 7.96 (d, J 7.8 Hz, 2H), 7.89 (d, J=3.8 Hz, 1H), 7.77 (d, J=8.0 Hz, 2H), 7.65-7.54 (m, 6H), 7.52-7.41 (m, 3H), 7.00 (d, J=3.6 Hz, 1H). ESI-MS m/z calc. 426.11505, found 427.3 (M+1)⁺; Retention time: 1.88 minutes; LC method A.

### Compound 30

### N-[7-tert-butyl-4-(3,5-dimethylphenyl)pyrido[2,3-d]pyrimidin-2-yl]benzenesulfonamide

### Step 1: 7-tert-Butyl-2-chloro-4-(3,5-dimethylphenyl)pyrido[2,3-d]pyrimidine

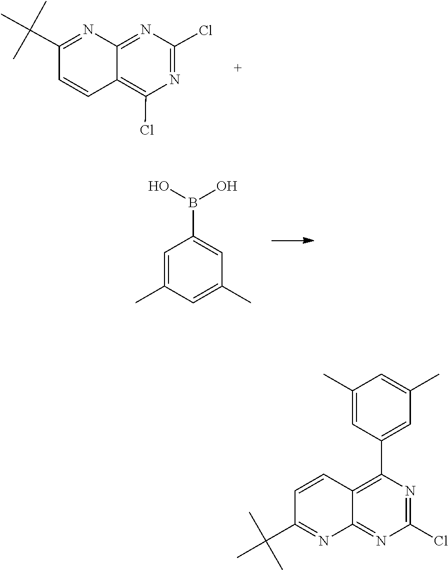

To 7-tert-butyl-2,4-dichloro-pyrido[2,3-d]pyrimidine (50 mg, 0.1952 mmol) was added (3,5-dimethylphenyl)boronic acid (31 mg, 0.2067 mmol), potassium carbonate (41 mg, 0.2967 mmol), Pd(PPh₃)₄ (12 mg, 0.01038 mmol) and toluene (2.3 mL). The reaction mixture was heated at 110° C. for 3.5 h. The solvent was evaporated under reduced pressure. The residue was dissolved in EtOAc and washed with water (×1) and brine (×1). The organic layer was dried over Na₂SO₄, filtered and concentrated to yield, 7-tert-butyl-2-chloro-4-(3,5-dimethylphenyl)pyrido[2,3-d] pyrimidine (32 mg, 50%). ESI-MS m/z calc. 325.13458, found 326.2 (M+1)⁺; Retention time: 0.84 minutes; LC method D.

### Step 2: N-[7-tert-butyl-4-(3,5-dimethylphenyl)pyrido[2,3-d]pyrimidin-2-yl]benzenesulfonamide

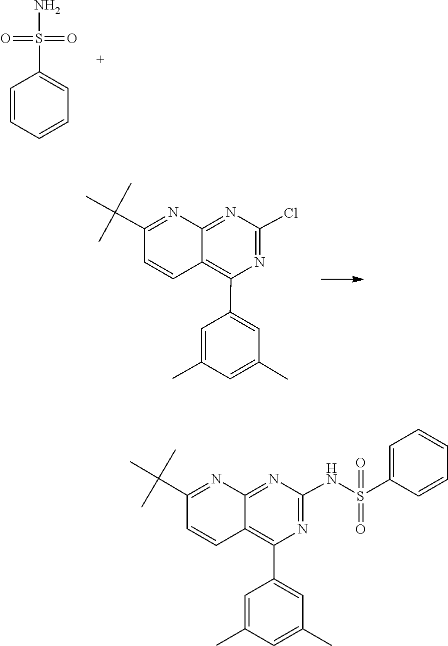 [SMILES: NS(=O)(=O)c1ccccc1 ; Cc1cc(C)cc(-c2nc(Cl)nc3nc(C(C)(C)C)ccc23)c1 ; Cc1cc(C)cc(-c2nc(NS(=O)(=O)c3ccccc3)nc3nc(C(C)(C)C)ccc23)c1]

To NaH (10.1 mg of 60% w/w, 0.2525 mmol) in DMA (1.0 mL) was added benzenesulfonamide (15.51 mg, 0.09867 mmol) very slowly. The reaction was stirred at rt for 15 min. 7-tert-Butyl-2-chloro-4-(3,5-dimethylphenyl)pyrido[2,3-d]pyrimidine (32 mg, 0.09821 mmol) was added to the reaction and stirred at 85° C. for 3 h. The reaction was cooled, poured onto ice water and EtOAc was added. The two layers were separated. The aqueous layer was extracted with EtOAc (×3). The combined organic layer was dried over Na₂SO₄, filtered and concentrated. The crude product was dissolved in DMSO, filtered and purified using a reverse phase HPLC C₁₈ column and a dual gradient run from 1-99% mobile phase B over 30 minutes [(Mobile phase A=H₂O (5 mM HCl). Mobile phase B=CH₃CN)] to yield N-[7-tert-butyl-4-(3,5-dimethylphenyl)pyrido[2,3-d]pyrimidin-2-yl]benzenesulfonamide (23.1 mg, 53%) as a white solid. ¹H NMR (400 MHz, DMSO-d₆) δ 12.10 (s, 1H), 8.22 (d, J=8.7 Hz, 1H), 7.98 (s, 2H), 7.72-7.39 (m, 4H), 7.27 (s, 1H), 7.05 (s, 2H), 2.37 (s, 6H), 1.39 (s, 9H). ESI-MS m/z calc. 446.17764, found 447.2 (M+1)⁺; Retention time: 1.98 minutes; LC method A.

### Compound 31

### N-(4-phenoxyquinazolin-2-yl)benzenesulfonamide

### Step 1: N-(4-chloroquinazolin-2-yl)benzenesulfonamide

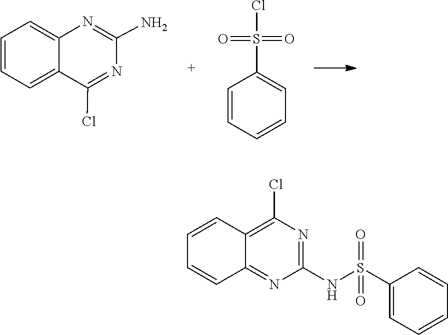 [SMILES: Nc1nc(Cl)c2ccccc2n1 ; O=S(=O)(Cl)c1ccccc1 ; O=S(=O)(Nc1nc(Cl)c2ccccc2n1)c1ccccc1]

To 4-chloroquinazolin-2-amine (200 mg, 1.114 mmol) in DMA (1.5 mL) was added NaH (45 mg of 60% w/w, 1.125 mmol) very slowly. The reaction was stirred at rt for 15 min. Benzenesulfonyl chloride (143 μL, 1.121 mmol) was added to the reaction and stirred at rt for 2 h. The reaction was quenched with MeOH. EtOAc was added to the reaction and washed with water (×1). The aqueous layer was extracted with EtOAc (×7). The combined organic layer was dried over Na₂SO₄, filtered and concentrated. The crude product was purified on 80 g of silica gel utilizing a gradient of 0-50% ethyl acetate in hexane to yield N-(4-chloroquinazolin-2-yl)benzenesulfonamide (46 mg, 13%) as a yellow solid. ESI-MS m/z calc. 319.01822, found 320.1 (M+1)⁺; Retention time: 1.35 minutes; LC method A.

### Step 2: N-(4-phenoxyquinazolin-2-yl)benzenesulfonamide

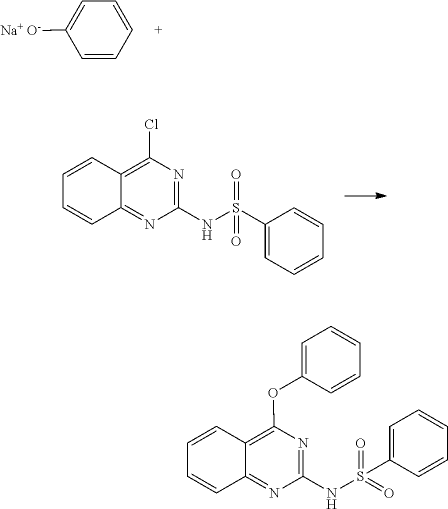 [SMILES: [Na+] ; [O-]c1ccccc1 ; O=S(=O)(Nc1nc(Cl)c2ccccc2n1)c1ccccc1 ; O=S(=O)(Nc1nc(Oc2ccccc2)c2ccccc2n1)c1ccccc1]

To N-(4-chloroquinazolin-2-yl)benzenesulfonamide (46 mg, 0.1439 mmol), sodium phenoxide (58 mg, 0.4996 mmol) and N,N-dimethyl formamide (1.2 mL) were added and the reaction was stirred at 110° C. for 25 min. The crude product was filtered and purified using a reverse phase HPLC C₁₈ column and a dual gradient run from 1-99% mobile phase B over 15 minutes [(Mobile phase A=H₂O (5 mM HCl). Mobile phase B=CH₃CN)] to yield N-(4-phenoxyquinazolin-2-yl)benzenesulfonamide (26.6 mg, 49%) as a white solid. ¹H NMR (400 MHz, DMSO-d₆) δ 13.01 (s, 1H), 8.14 (d, J=8.3 Hz, 1H), 7.97-7.80 (m, 1H), 7.61 (t, J=7.8 Hz, 2H), 7.57-6.87 (m, 10H). ESI-MS m/z calc. 377.0834, found 378.2 (M+1)⁺; Retention time: 1.48 minutes; LC method A.

### Compound 32

### 3-Amino-N-(4,7-diphenylpyrrolo[2,3-d]pyrimidin-2-yl)benzenesulfonamide

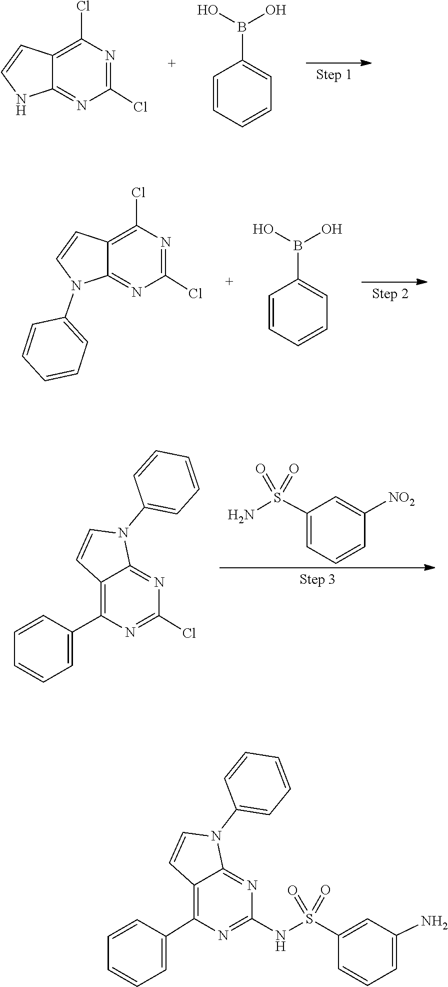

### Step 1: 2,4-Dichloro-7-phenyl-pyrrolo[2,3-d]pyrimidine

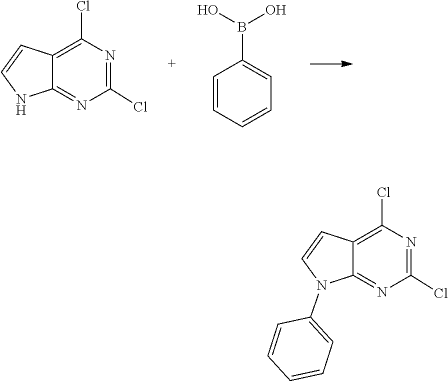 [SMILES: Clc1nc(Cl)c2cc[nH]c2n1 ; OB(O)c1ccccc1 ; Clc1nc(Cl)c2ccn(-c3ccccc3)c2n1]

To a mixture of 2,4-dichloro-7H-pyrrolo[2,3-d]pyrimidine (500 mg, 2.659 mmol), phenylboronic acid (approximately 648.4 mg, 5.318 mmol), pyridine N-oxide (approximately 278.2 mg, 2.925 mmol), TEA (approximately 1.346 g, 1.854 mL, 13.30 mmol) and pyridine (approximately 1.052 g, 1.076 mL, 13.30 mmol) was added diacetoxycopper (approximately 579.6 mg, 3.191 mmol) and the reaction mixture stirred open to the air for 72 hours. The reaction mixture was poured into water and extracted with EtOAc (3×). Organics were combined, washed with 0.1 HCl (3×), water and brine then dried over Na₂SO₄ and evaporated to dryness. Purification by column chromatography (40 g silica; 0-30% EtOAc in hexanes) gave 2,4-dichloro-7-phenyl-pyrrolo[2,3-d]pyrimidine (622 mg, 89%) as a tan solid. ESI-MS m/z calc. 263.0017, found 264.1 (M+1)⁺; Retention time: 0.7 minutes; LC method A.

### Step 2: 2-Chloro-4,7-diphenyl-pyrrolo[2,3-d]pyrimidine

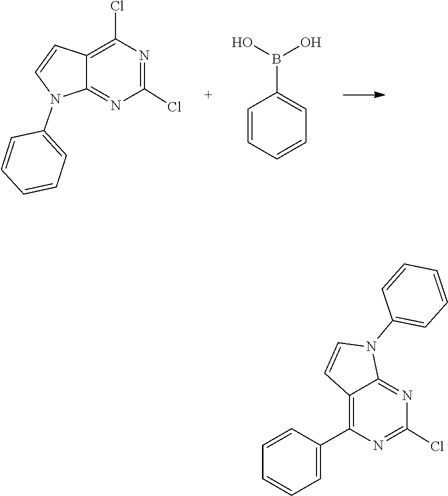 [SMILES: Clc1nc(Cl)c2ccn(-c3ccccc3)c2n1 ; OB(O)c1ccccc1 ; Clc1nc(-c2ccccc2)c2ccn(-c3ccccc3)c2n1]

A mixture of 2,4-dichloro-7-phenyl-pyrrolo[2,3-d]pyrimidine (200 mg, 0.7573 mmol), 2,4-dichloro-7-phenyl-pyrrolo[2,3-d]pyrimidine (200 mg, 0.7573 mmol), sodium carbonate (approximately 946.5 μL of 2 M, 1.893 mmol) and Pd(II)DPPF (approximately 27.70 mg, 0.03786 mmol) was stirred at 70° C. for 6 hours. The reaction mixture was diluted with water and extracted with EtOAc (3×). Organics combined and purification by column chromatography (24 g silica; 0-30% EtOAc in hexanes) gave 2-chloro-4,7-diphenyl-pyrrolo[2,3-d]pyrimidine (151 mg, 65%) as a reddish semisolid. ESI-MS m/z calc. 305.07196, found 306.2 (M+1)⁺; Retention time: 0.77 minutes; LC method D.

### Step 3: 3-Amino-N-(4,7-diphenylpyrrolo[2,3-d]pyrimidin-2-yl)benzenesulfonamide

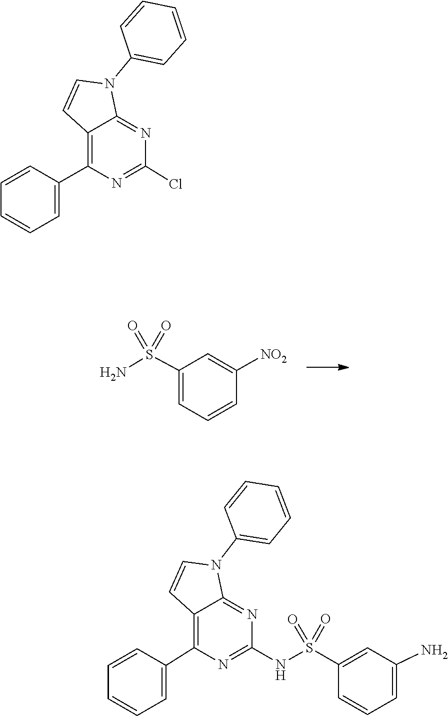 [SMILES: Clc1nc(-c2ccccc2)c2ccn(-c3ccccc3)c2n1 ; NS(=O)(=O)c1cccc([N+](=O)[O-])c1 ; Nc1cccc(S(=O)(=O)Nc2nc(-c3ccccc3)c3ccn(-c4ccccc4)c3n2)c1]

A solution of 2-chloro-4,7-diphenyl-pyrrolo[2,3-d]pyrimidine (15 mg, 0.04906 mmol), (5-diphenylphosphanyl-9,9-dimethyl-xanthen-4-yl)-diphenyl-phosphane (approximately 8.517 mg, 0.01472 mmol), palladium(II)diacetate (approximately 1.652 mg, 0.007359 mmol), 3-nitrobenzenesulfonamide (approximately 29.76 mg, 0.1472 mmol) and Cs₂CO₃ (approximately 31.97 mg, 0.09812 mmol) in dioxane (450.0 μL) was stirred while nitrogen was being bubbled through the mixture for 20 min, then the reaction mixture was stirred at 100° C. for 20 min. The reaction mixture was diluted with water and made acidic with HCl and extracted with EtOAc (3×). The organics were combined, filtered through a short plug of silica and evaporated to dryness. The residue and 10% Pd/C (10 mg of 10% w/w, 0.009397 mmol) was taken up in MeOH (1 mL) and EtOAc (0.5 mL) and stirred under an atmosphere of hydrogen for 4 hours. At this time more 10% Pd/C (10 mg of 10% w/w, 0.009397 mmol) was added and the reaction mixture stirred under an atmosphere of hydrogen for 2 hours. The reaction mixture was diluted with EtOAc and filtered through a short plug of silica and evaporated to dryness. Purification by column chromatography (4 g silica; 0-30% EtOAc in hexanes) gave 3-amino-N-(4,7-diphenylpyrrolo[2,3-d]pyrimidin-2-yl)benzenesulfonamide as a white solid. ESI-MS m/z calc. 441.12595, found 442.2 (M+1)⁺; Retention time: 1.64 minutes; LC method A.

### Compound 33

### N-[6-tert-butyl-1-(2,4,6-trimethylphenyl)pyrazolo[3,4-b]pyridin-3-yl]benzenesulfonamide

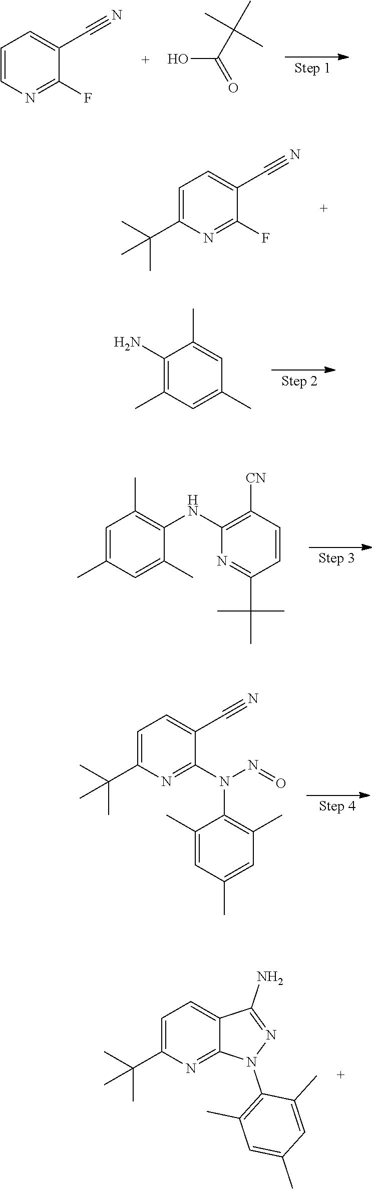

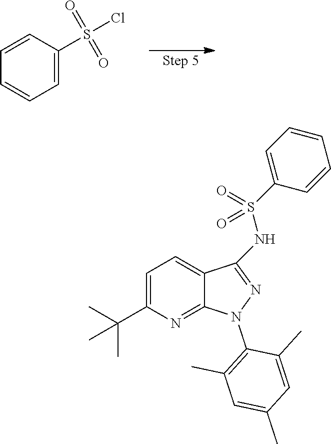 [SMILES: O=S(=O)(Cl)c1ccccc1 ; Cc1cc(C)c(-n2nc(NS(=O)(=O)c3ccccc3)c3ccc(C(C)(C)C)nc32)c(C)c1]

### Step 1: 6-tert-Butyl-2-fluoro-pyridine-3-carbonitrile

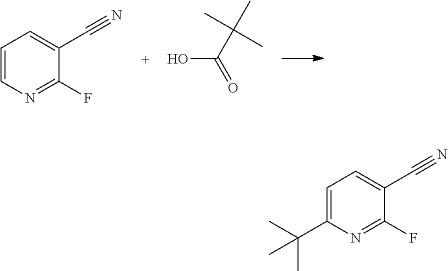

2,2-Dimethylpropanoic acid (approximately 29.28 g, 16.46 mL, 286.7 mmol), 2-fluoropyridine-3-carbonitrile (7.00 g, 57.33 mmol), silver nitrate (approximately 2.434 g, 14.33 mmol) were suspended in a solution of sulfuric acid (7.947 mL) in H₂O (79.47 mL). A solution of ammonium persulfate (approximately 24.22 g, 114.7 mmol) in water (158.9 mL) was added dropwise through an addition funnel. The mixture was stirred at room temperature for 2 days. The pH of reaction mixture was adjusted to ˜8-9 with concentrated NH₄OH (˜60 ml) and extracted with ethyl acetate (3×100 ml). The combined organic layers were extracted with brine, dried over Na₂SO₄, and concentrated. The crude was purified on silica using ethyl acetate/hexane. The fractions were concentrated to give a clear thin oil (5.5 grams). It was dissolved in EtOAc (75 mL) and washed with aqueous NaOH (1 N, 4×75 mL). The organic layer was dried over sodium sulfate, filtered and concentrated under reduced pressure to provide 6-tert-butyl-2-fluoro-pyridine-3-carbonitrile (5.37 g, 53%) as a clear colorless oil that crystallized upon standing. ESI-MS m/z calc. 178.09062, found 179.1 (M+1)⁺; Retention time: 1.3 minutes (UPLC, 10-99% gradient over 3 min of ACN in water containing TFA).

### Step 2: 6-tert-Butyl-2-(2,4,6-trimethylanilino)pyridine-3-carbonitrile

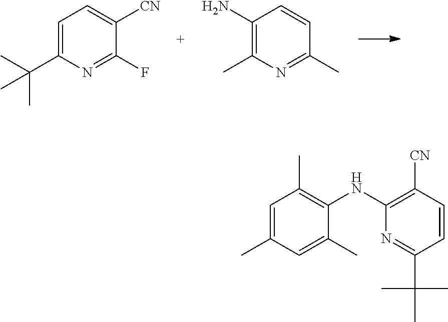

To sodium hydride (4.1 g of 60% w/w, 102.5 mmol) in DMA (120.0 mL) was added 2,4,6-trimethylaniline (14 mL, 99.71 mmol) very slowly. The reaction was stirred at rt for 40 min. 6-tert-butyl-2-fluoro-pyridine-3-carbonitrile (3 g, 16.83 mmol) was added to the reaction and stirred at 100° C. for 2 h. The reaction mixture was cooled and diluted with ethyl acetate (200 mL) and water (150 mL). The two layers were separated. The organic layer was washed with water (3×150 mL). The organic layer was dried over Na₂SO₄, filtered and concentrated. The crude product was purified on 330 g of silica gel utilizing a gradient of 1-15% ethyl acetate in hexane to yield 6-tert-butyl-2-(2,4,6-trimethylanilino)pyridine-3-carbonitrile (2.54 g, 51%) as a yellow solid. ¹H NMR (400 MHz, DMSO-d₆) δ 8.35 (s, 1H), 7.85 (d, J=8.1 Hz, 1H), 6.89 (s, 2H), 6.72 (d, J=8.1 Hz, 1H), 2.25 (s, 3H), 2.06 (s, 6H), 1.05 (s, 9H). ESI-MS m/z calc. 293.1892, found 294.2 (M+1)⁺; Retention time: 2.31 minutes (LC method A).

### Step 3: N-(6-tert-butyl-3-cyano-2-pyridyl)-N-(2,4,6-trimethylphenyl)nitrous amide

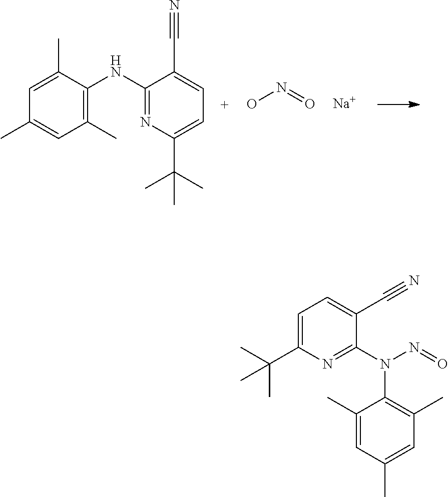

To a solution of 6-tert-butyl-2-(2,4,6-trimethylanilino)pyridine-3-carbonitrile (1.3 g, 4.431 mmol) in AcOH (40 mL) was added sodium nitrite (approximately 3.057 g, 1.410 mL, 44.31 mmol) in water (10 mL) at 0° C. The mixture was stirred at rt for 2 h. Another 5 more equivalents of sodium nitrite was added and the reaction was heated to 50° C. After evaporation of the solvents, the reaction was diluted with ethyl acetate/water. The organic layer was separated, dried over Na₂SO₄ and concentrated to afford N-(6-tert-butyl-3-cyano-2-pyridyl)-N-(2,4,6-trimethylphenyl)nitrous amide (1.56 g, 109%). ESI-MS m/z calc. 322.17935, found 324.26 (M+1)⁺; Retention time: 0.83 minutes (LC method D).

### Step 4: 6-tert-Butyl-1-(2,4,6-trimethylphenyl)pyrazolo[3,4-b]pyridin-3-amine

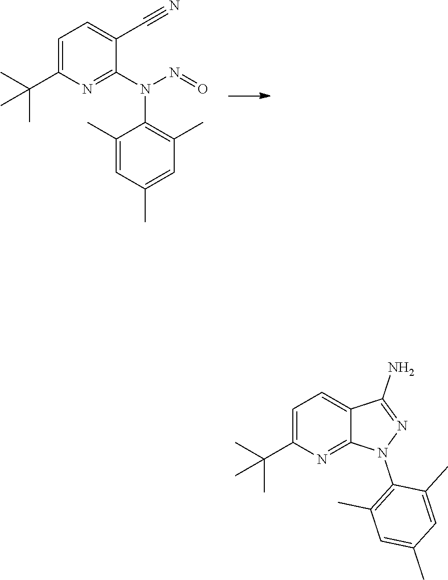 [SMILES: Cc1cc(C)c(N(N=O)c2nc(C(C)(C)C)ccc2C#N)c(C)c1 ; Cc1cc(C)c(-n2nc(N)c3ccc(C(C)(C)C)nc32)c(C)c1]

To a solution of crude N-(6-tert-butyl-3-cyano-2-pyridyl)-N-(2,4,6-trimethylphenyl)nitrous amide (1.429 g, 4.431 mmol) in AcOH (20 mL) was added Zn (approximately 2.898 g, 406.3 μL, 44.31 mmol) in small portions over 15 minutes and the reaction was stirred at rt for 30 min. The reaction was filtered through a pad of Celite and the pad was washed with ethyl acetate. Evaporation and purification by silica gel chromatography using 0-60% ethyl acetate in hexane gave 6-tert-butyl-1-(2,4,6-trimethylphenyl)pyrazolo[3,4-b]pyridin-3-amine (850 mg, 62%) as an off-white solid. ESI-MS m/z calc. 308.2001, found 309.28 (M+1)⁺; Retention time: 1.97 minutes. (LC method E).

### Step 5: N-[6-tert-butyl-1-(2,4,6-trimethylphenyl)pyrazolo[3,4-b]pyridin-3-yl]benzenesulfonamide

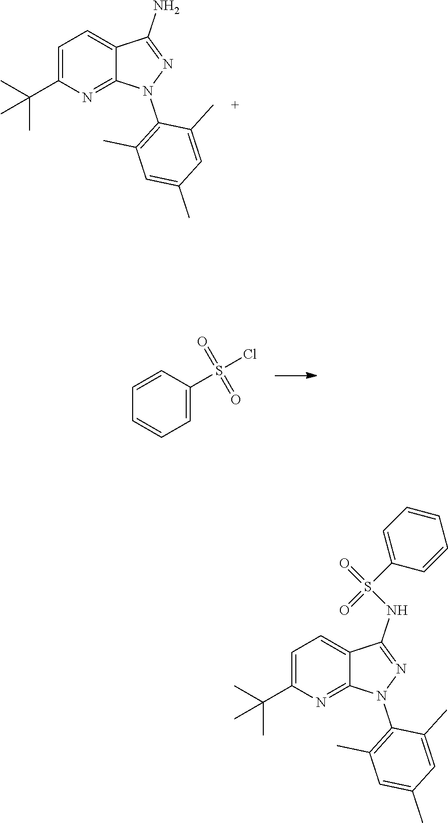 [SMILES: Cc1cc(C)c(-n2nc(N)c3ccc(C(C)(C)C)nc32)c(C)c1 ; O=S(=O)(Cl)c1ccccc1 ; Cc1cc(C)c(-n2nc(NS(=O)(=O)c3ccccc3)c3ccc(C(C)(C)C)nc32)c(C)c1]

To a solution of 6-tert-butyl-1-(2,4,6-trimethylphenyl)pyrazolo[3,4-b]pyridin-3-amine (17 mg, 0.05512 mmol) in pyridine (2 mL) was added benzenesulfonyl chloride (approximately 29.21 mg, 21.11 μL, 0.1654 mmol) at 0° C. and the reaction was stirred at rt for 30 min. The reaction was concentrated and the residue was dissolved in DMSO and was purified by reverse phase HPLC using 1-99% CH₃CN in water to afford N-[6-tert-butyl-1-(2,4,6-trimethylphenyl)pyrazolo[3,4-b]pyridin-3-yl]benzenesulfonamide (14 mg, 57%). ESI-MS m/z calc. 448.1933, found 449.34 (M+1)⁺; Retention time: 2.31 minutes. (LC method E).

### Compound 34

### N-[6-tert-butyl-1-(2,4,6-trimethylphenyl)pyrazolo[3,4-b]pyridin-3-yl]-3-(dimethylamino)benzenesulfonamide

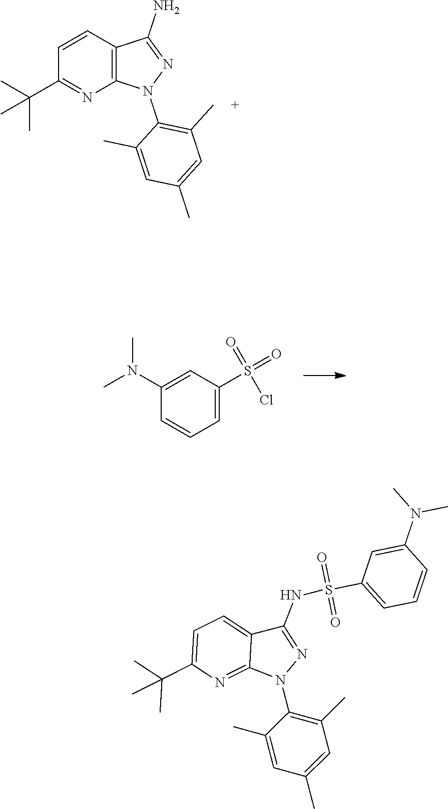 [SMILES: Cc1cc(C)c(-n2nc(N)c3ccc(C(C)(C)C)nc32)c(C)c1 ; CN(C)c1cccc(S(=O)(=O)Cl)c1 ; Cc1cc(C)c(-n2nc(NS(=O)(=O)c3cccc(N(C)C)c3)c3ccc(C(C)(C)C)nc32)c(C)c1]

A solution of 6-tert-butyl-1-(2,4,6-trimethylphenyl)pyrazolo[3,4-b]pyridin-3-amine (15 mg, 0.04863 mmol) in pyridine (0.2 mL) was prepared and cooled down to 0° C. The solution was added to 3-(dimethylamino)benzenesulfonyl chloride (approximately 42.73 mg, 0.1945 mmol) at 0° C. and the mixture was heated at 100° C. for 45 min. The reaction mixture was filtered and purified on reverse phase HPLC (HCl modifier, 30-99% ACN-H₂O) to give N-[6-tert-butyl-1-(2,4,6-trimethylphenyl)pyrazolo[3,4-b]pyridin-3-yl]-3-(dimethylamino)benzenesulfonamide (15.5 mg, 64%). ESI-MS m/z calc. 491.2355, found 492.0 (M+1)⁺; Retention time: 2.33 minutes; LC method A. ¹H NMR (400 MHz, DMSO) δ 10.80 (s, 1H), 8.21 (d, J=8.6 Hz, 1H), 7.37 (d, J=8.6 Hz, 1H), 7.27 (t, J=8.0 Hz, 1H), 7.00 (s, 2H), 6.96 (d, J=7.7 Hz, 1H), 6.91 (s, 1H), 6.88 (d, J=8.3 Hz, 1H), 2.78 (s, 6H), 2.31 (s, 3H), 1.65 (s, 6H), 1.26 (s, 9H).

### Compound 35

### N-[6-tert-butyl-1-(2,4,6-trimethylphenyl)pyrazolo[3,4-b]pyridin-3-yl]-1H-pyrazole-4-sulfonamide

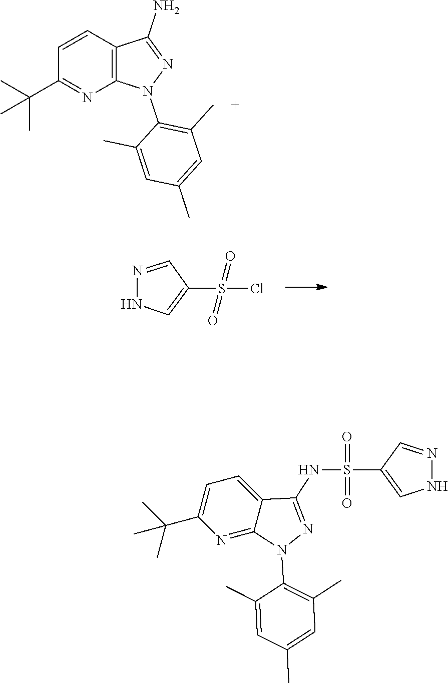 [SMILES: Cc1cc(C)c(-n2nc(N)c3ccc(C(C)(C)C)nc32)c(C)c1 ; O=S(=O)(Cl)c1cn[nH]c1 ; Cc1cc(C)c(-n2nc(NS(=O)(=O)c3cn[nH]c3)c3ccc(C(C)(C)C)nc32)c(C)c1]

To a solution of 6-tert-butyl-1-(2,4,6-trimethylphenyl)pyrazolo[3,4-b]pyridin-3-amine (17 mg, 0.05512 mmol) in pyridine (2 mL) was added benzenesulfonyl chloride (approximately 29.21 mg, 21.11 μL, 0.1654 mmol) at 0° C. and the reaction was stirred at rt for 30 min. The reaction was concentrated and the residue was dissolved in DMSO and was purified by reverse phase HPLC using 1-99% CH₃CN in water to afford N-[6-tert-butyl-1-(2,4,6-trimethylphenyl)pyrazolo[3,4-b]pyridin-3-yl]benzenesulfonamide (14 mg, 57%). ESI-MS m/z calc. 448.1933, found 449.34 (M+1)⁺; Retention time: 2.31 minutes. (LC method E).

The compound was prepared in a manner analogous to that described above using commercially available 1H-pyrazole-4-sulfonyl chloride. N-[6-tert-butyl-1-(2,4,6-trimethylphenyl)pyrazolo[3,4-b]pyridin-3-yl]-1H-pyrazole-4-sulfonamide (12.7 mg, 59%). ESI-MS m/z calc. 438.1838, found 439.0 (M+1)⁺; Retention time: 1.94 minutes (LC method A). ¹H NMR (400 MHz, DMSO) δ 13.50 (s, 1H), 10.75 (s, 1H), 8.42-8.04 (m, 2H), 7.68 (s, 1H), 7.38 (d, J=8.3 Hz, 1H), 7.03 (s, 2H), 2.32 (s, 3H), 1.73 (s, 6H), 1.26 (s, 9H).

### Compound 36

### 4-Amino-N-[6-tert-butyl-1-(2,4,6-trimethylphenyl)pyrazolo[3,4-b]pyridin-3-yl]benzenesulfonamide

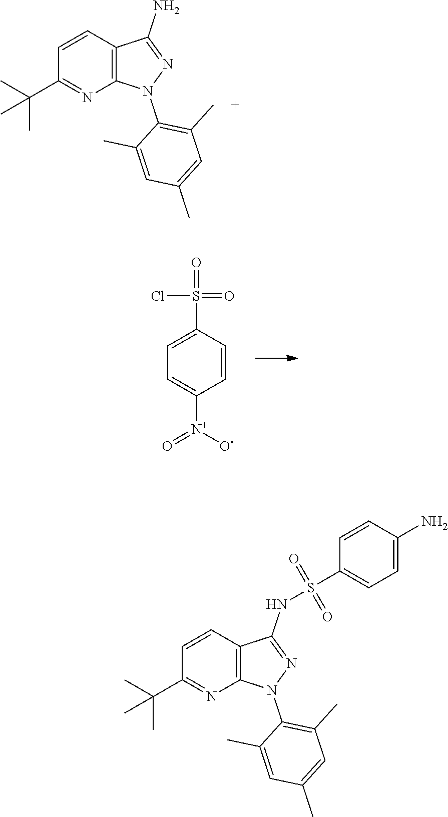

The compound was prepared in a manner analogous to that described above using commercially available 4-nitrobenzenesulfonyl chloride. The nitro group was reduced using Fe/HCl (10eq/10eq) in ethanol/THF (150 μl/300 μl) at 90° C. for 30 min. 4-amino-N-[6-tert-butyl-1-(2,4,6-trimethylphenyl)pyrazolo[3,4-b]pyridin-3-yl]benzenesulfonamide (12.8 mg, 60%). ESI-MS m/z calc. 463.2042, found 464.0 (M+1)⁺; Retention time: 1.92 minutes; LCMS Method: (LC method E). ¹H NMR (400 MHz, DMSO) δ 10.45 (s, 1H), 8.22 (d, J=8.5 Hz, 1H), 7.34 (dd, J=13.7, 8.6 Hz, 3H), 7.00 (s, 2H), 6.48 (d, J=8.6 Hz, 2H), 5.93 (s, 2H), 2.32 (s, 3H), 1.70 (s, 6H), 1.26 (s, 9H).

### Compound 37

### N-[6-tert-Butyl-1-(3,5-dimethylphenyl)pyrazolo[3,4-b]pyridin-3-yl]benzenesulfonamide

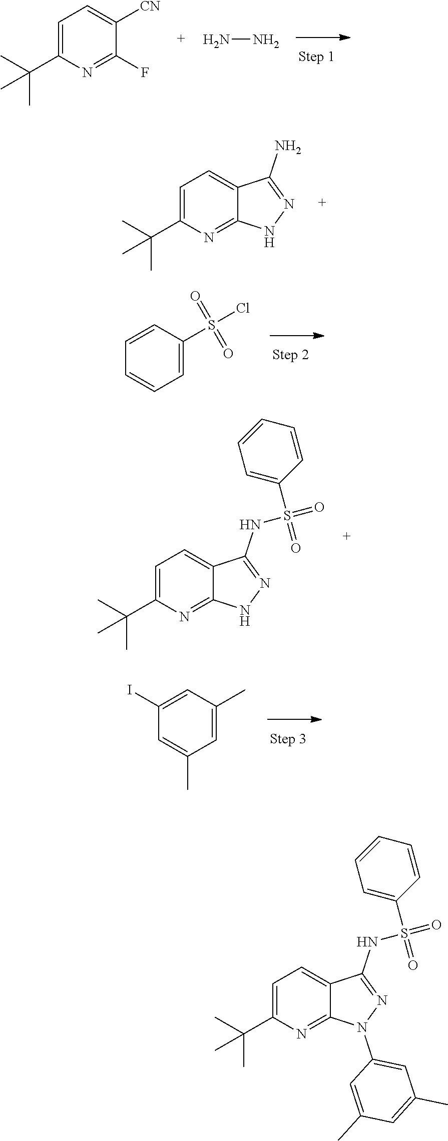

### Step 1: 6-tert-Butyl-1H-pyrazolo[3,4-b]pyridin-3-amine

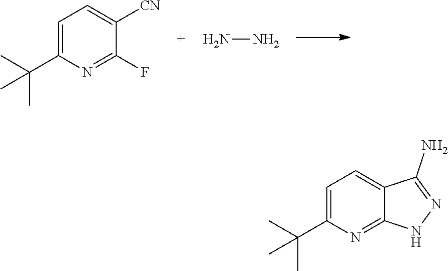

To a solution of 6-tert-butyl-2-fluoro-pyridine-3-carbonitrile (1 g, 5.611 mmol) in EtOH (10 mL) was added Hydrazine hydrate (approximately 702.3 mg, 682.5 μL, 14.03 mmol) and the reaction was heated at 85° C. for 1 h. The solvents were evaporated and the residue was purified by silica gel using 0-50% ethyl acetate in DCM to afford 6-tert-butyl-1H-pyrazolo[3,4-b]pyridin-3-amine (400 mg, 37%) ESI-MS m/z calc. 190.12184, found 191.05 (M+1)⁺; Retention time: 0.73 minutes (LC method A).

### Step 2: N-(6-tert-Butyl-1H-pyrazolo[3,4-b]pyridin-3-yl)benzenesulfonamide

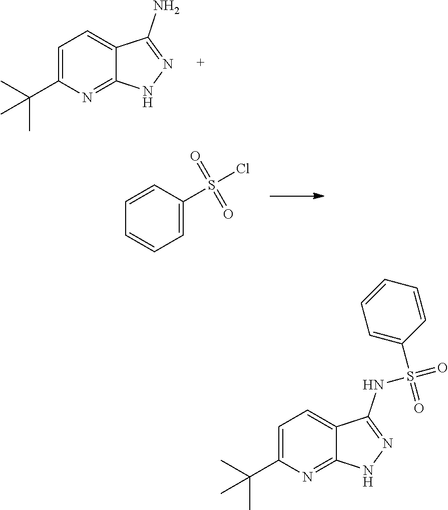 [SMILES: CC(C)(C)c1ccc2c(N)n[nH]c2n1 ; O=S(=O)(Cl)c1ccccc1 ; CC(C)(C)c1ccc2c(NS(=O)(=O)c3ccccc3)n[nH]c2n1]

To a solution of 6-tert-butyl-1H-pyrazolo[3,4-b]pyridin-3-amine (400 mg, 2.103 mmol) in pyridine (3 mL) at 0° C. was added benzenesulfonyl chloride (approximately 371.4 mg, 268.4 μL, 2.103 mmol) very slowly. The mixture was stirred at rt for 1 h. After evaporation, ethyl acetate and water were added. The organic layer was concentrated, and the residue was sonicated with ether:hexanes (1:1). The precipitate was filtered to afford N-(6-tert-butyl-1H-pyrazolo[3,4-b]pyridin-3-yl)benzenesulfonamide (590 mg, 85%) ESI-MS m/z calc. 330.11505, found 331.16 (M+1)⁺; Retention time: 0.59 minutes (LC method D).

### Step 3: N-[6-tert-Butyl-1-(3,5-dimethylphenyl)pyrazolo[3,4-b]pyridin-3-yl]benzenesulfonamide

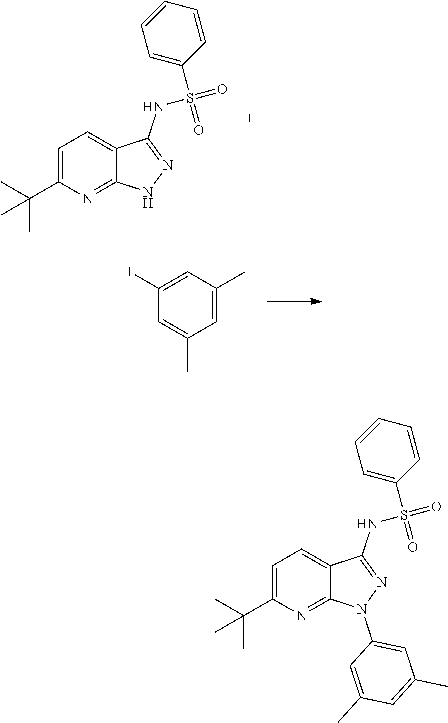

To a mixture of N-(6-tert-butyl-1H-pyrazolo[3,4-b]pyridin-3-yl)benzenesulfonamide (20 mg, 0.06053 mmol), 1-iodo-3,5-dimethyl-benzene (15 mg, 0.06464 mmol), Cs₂CO₃ (50 mg, 0.1535 mmol), quinolin-8-ol (7 mg, 0.04822 mmol), and iodocopper (6 mg, 0.03150 mmol) was added t-BuOH (1.500 mL) and the reaction was flushed with nitrogen thoroughly. It was sealed and was heated at 105° C. overnight. It was filtered using a pad of Celite using methanol/ethyl acetate (1:1). The filtrate was collected, concentrated and the residue was dissolved in DMSO and was purified by reverse phase HPLC using 1-99% CH₃CN in water to afford N-[6-tert-butyl-1-(3,5-dimethylphenyl)pyrazolo[3,4-b]pyridin-3-yl]benzenesulfonamide (11.2 mg). ESI-MS m/z calc. 434.17764, found 435.3 (M+1)⁺; Retention time: 2.38 minutes (LC method E).

### Compound 38

### N-[6-tert-butyl-1-(3-chlorophenyl)pyrazolo[3,4-b]pyridin-3-yl]benzenesulfonamide

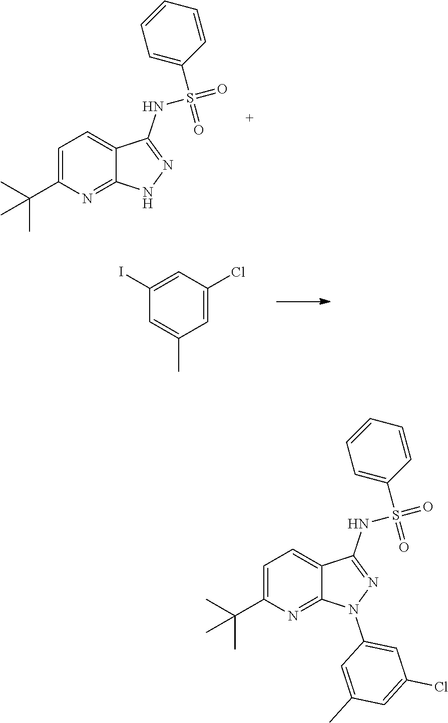

To a mixture of N-(6-tert-butyl-1H-pyrazolo[3,4-b]pyridin-3-yl)benzenesulfonamide (20 mg, 0.06053 mmol), 1-chloro-3-iodo-benzene (14 mg, 0.05871 mmol), Cs₂CO₃ (50 mg, 0.1535 mmol), quinolin-8-ol (approximately 7.000 mg, 0.04822 mmol) and iodocopper (6 mg, 0.03150 mmol) was added t-BuOH (1.500 mL) and reaction was flushed with nitrogen thoroughly. It was sealed and was heated at 105° C. overnight. It was filtered using a pad of Celite using methanol/ethyl acetate (1:1). The filtrate was collected, concentrated and the residue was dissolved in DMSO which was purified by reverse phase HPLC using 1-99% CH₃CN in water to afford N-[6-tert-butyl-1-(3-chlorophenyl)pyrazolo[3,4-b]pyridin-3-yl]benzenesulfonamide (15.1 mg, 56%). ESI-MS m/z calc. 440.10736, found 441.3 (M+1)⁺; Retention time: 2.36 minutes (LC method E). ¹H NMR (400 MHz, DMSO) δ 11.51 (s, 1H), 8.38 (t, J=2.1 Hz, 1H), 8.30 (d, J=8.6 Hz, 1H), 8.15 (ddd, J=8.6, 2.2, 1.0 Hz, 1H), 7.99-7.91 (m, 2H), 7.71-7.47 (m, 5H), 7.31 (ddd, J=8.0, 2.1, 0.9 Hz, 1H), 1.41 (s, 9H).

### Compound 39

### N-(6-tert-butyl-1-isopropyl-pyrazolo[3,4-b]pyridin-3-yl)benzenesulfonamide

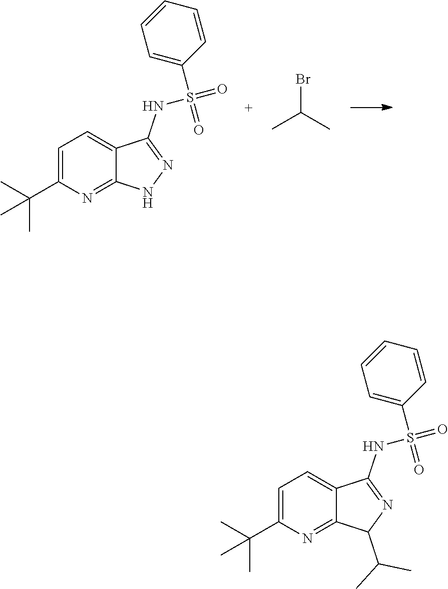

To a solution of N-(6-tert-butyl-1H-pyrazolo[3,4-b]pyridin-3-yl)benzenesulfonamide (20 mg, 0.06053 mmol) in DMF (1 mL) was added K₂CO₃ (9 mg, 0.06512 mmol) and stirred for 10 min at rt. To this solution was added 2-bromopropane (8 mg, 0.06504 mmol) and the reaction mixture was heated to 60° C. for 4 h. The reaction mixture was filtered and subjected to HPLC purification using 1-99% ACN in water (0.05% HCl modifier) over 15 min. The desired fractions were concentrated to produce t N-(6-tert-butyl-1-isopropyl-pyrazolo[3,4-b]pyridin-3-yl)benzenesulfonamide (3.7 mg) as an off white powder. ¹H NMR (400 MHz, DMSO) δ 13.65 (s, 1H), 7.93 (dd, J=8.6, 0.8 Hz, 1H), 7.89-7.82 (m, 2H), 7.74-7.66 (m, 1H), 7.66-7.59 (m, 2H), 7.41 (d, J=8.5 Hz, 1H), 4.40 (p, J=6.6 Hz, 1H), 1.40 (s, 9H), 0.99 (d, J=6.7 Hz, 6H). ESI-MS m/z calc. 372.162, found 373.3 (M+1)⁺; Retention time: 1.86 minutes (LC method E).

### Compounds 40-69

The compounds in the following Tables 9 and 10 were prepared in a manner analogous to that described above using commercially available reagents and intermediates described herein.

TABLE 9
Characterization data for Compounds 40, 46-68

| | | LCMS | | | |
|---|---|---|---|---|---|
| Cmpd | | Rt | Calc. | | LCMS |
| No. | Structure | (min) | mass | M + 1 | Method |
| 40 | 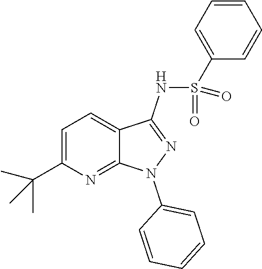 [SMILES: CC(C)(C)c1ccc2c(NS(=O)(=O)c3ccccc3)nn(-c3ccccc3)c2n1] | 2.19 | 406.146 | 407.5 | A |
| 41 | 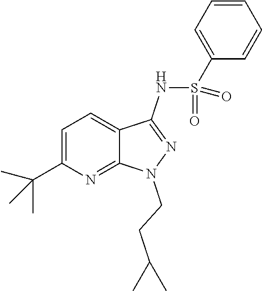 [SMILES: CC(C)CCn1nc(NS(=O)(=O)c2ccccc2)c2ccc(C(C)(C)C)nc21] | 2.15 | 400.193 | 401.3 | A |
| 42 | 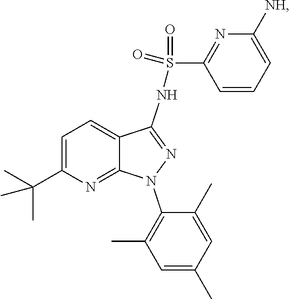 | 2.07 | 464.199 | 465.28 | A |
| 43 | 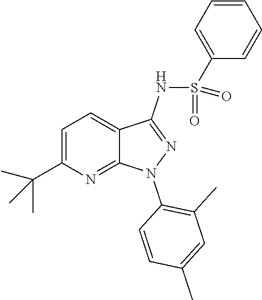 [SMILES: Cc1ccc(-n2nc(NS(=O)(=O)c3ccccc3)c3ccc(C(C)(C)C)nc32)c(C)c1] | 2.25 | 434.178 | 435.3 | D |
| 44 | 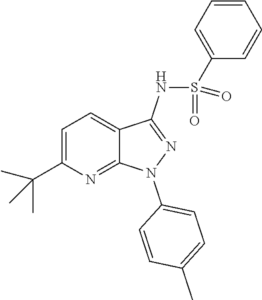 [SMILES: Cc1ccc(-n2nc(NS(=O)(=O)c3ccccc3)c3ccc(C(C)(C)C)nc32)cc1] | 2.34 | 420.162 | 421.3 | A |
| 45 | 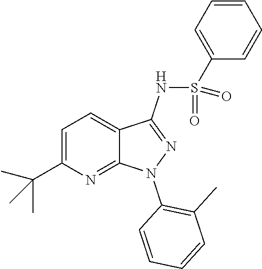 [SMILES: Cc1ccccc1-n1nc(NS(=O)(=O)c2ccccc2)c2ccc(C(C)(C)C)nc21] | 2.16 | 420.162 | 421.3 | A |
| 46 | 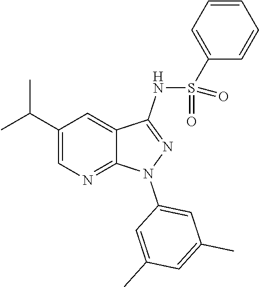 [SMILES: Cc1cc(C)cc(-n2nc(NS(=O)(=O)c3ccccc3)c3cc(C(C)C)cnc32)c1] | 2.2 | 420.162 | 421.4 | A |
| 47 | 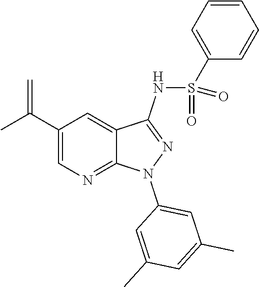 [SMILES: C=C(C)c1cnc2c(c1)c(NS(=O)(=O)c1ccccc1)nn2-c1cc(C)cc(C)c1] | 2.16 | 418.146 | 419.3 | A |
| 48 | 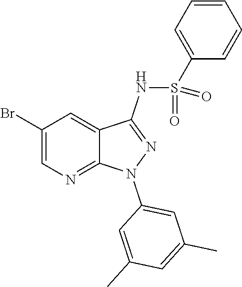 [SMILES: Cc1cc(C)cc(-n2nc(NS(=O)(=O)c3ccccc3)c3cc(Br)cnc32)c1] | 21.6 | 456.026 | 457.2 | A |
| 49 | 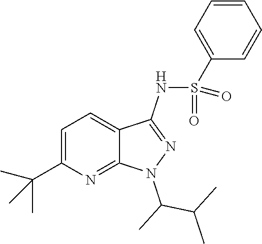 [SMILES: CC(C)C(C)n1nc(NS(=O)(=O)c2ccccc2)c2ccc(C(C)(C)C)nc21] | 1.64 | 400.193 | 401.4 | A |
| 50 | 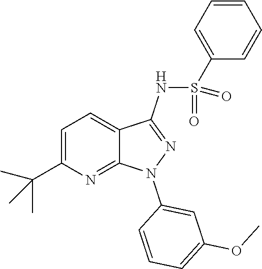 [SMILES: COc1cccc(-n2nc(NS(=O)(=O)c3ccccc3)c3ccc(C(C)(C)C)nc32)c1] | 2.17 | 436.157 | 437.3 | A |
| 51 | 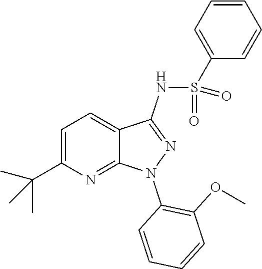 [SMILES: COc1ccccc1-n1nc(NS(=O)(=O)c2ccccc2)c2ccc(C(C)(C)C)nc21] | 1.99 | 436.157 | 437.3 | A |
| 52 | 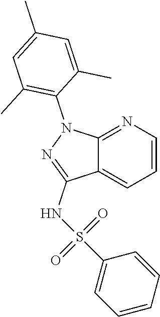 [SMILES: Cc1cc(C)c(-n2nc(NS(=O)(=O)c3ccccc3)c3cccnc32)c(C)c1] | 1.7 | 392.131 | 393 | A |
| 53 | 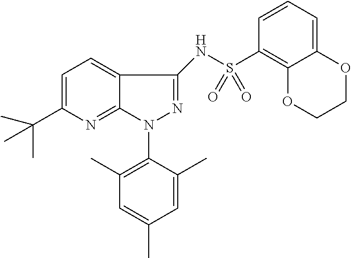 [SMILES: Cc1cc(C)c(-n2nc(NS(=O)(=O)c3cccc4c3OCCO4)c3ccc(C(C)(C)C)nc32)c(C)c1] | 2.25 | 506.199 | 507 | A |
| 54 | 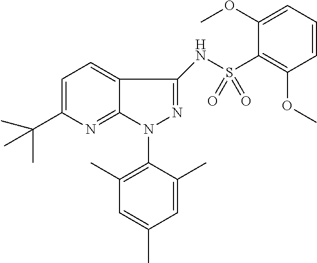 [SMILES: COc1cccc(OC)c1S(=O)(=O)Nc1nn(-c2c(C)cc(C)cc2C)c2nc(C(C)(C)C)ccc12] | 2.17 | 508.214 | 509.6 | A |
| 55 | 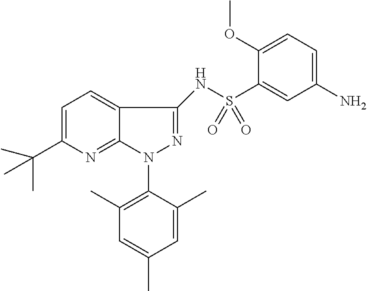 [SMILES: COc1ccc(N)cc1S(=O)(=O)Nc1nn(-c2c(C)cc(C)cc2C)c2nc(C(C)(C)C)ccc12] | 1.71 | 493.215 | 494 | A |
| 56 | 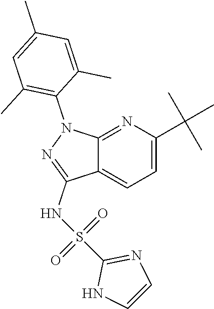 [SMILES: Cc1cc(C)c(-n2nc(NS(=O)(=O)c3ncc[nH]3)c3ccc(C(C)(C)C)nc32)c(C)c1] | 1.92 | 438.184 | 439 | A |
| 57 | 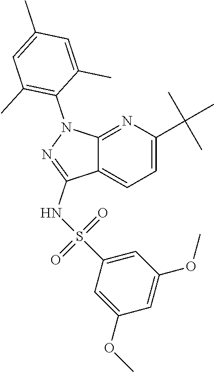 [SMILES: COc1cc(OC)cc(S(=O)(=O)Nc2nn(-c3c(C)cc(C)cc3C)c3nc(C(C)(C)C)ccc23)c1] | 2.32 | 508.214 | 509 | A |
| 58 | 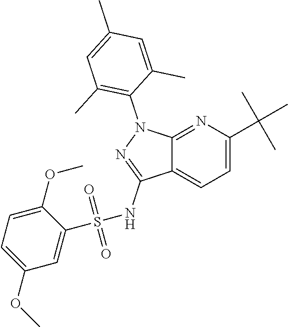 [SMILES: COc1ccc(OC)c(S(=O)(=O)Nc2nn(-c3c(C)cc(C)cc3C)c3nc(C(C)(C)C)ccc23)c1] | 2.29 | 508.214 | 509 | A |
| 59 | 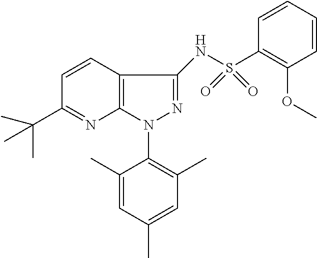 [SMILES: COc1ccccc1S(=O)(=O)Nc1nn(-c2c(C)cc(C)cc2C)c2nc(C(C)(C)C)ccc12] | 2.1 | 478.204 | 479 | A |
| 60 | 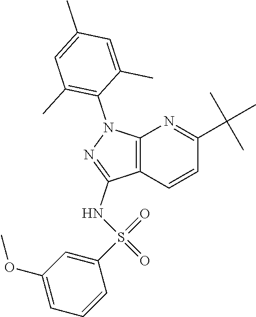 [SMILES: COc1cccc(S(=O)(=O)Nc2nn(-c3c(C)cc(C)cc3C)c3nc(C(C)(C)C)ccc23)c1] | 2.13 | 478.204 | 479 | A |
| 61 | 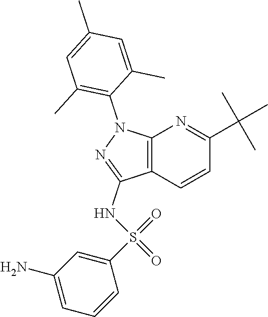 [SMILES: Cc1cc(C)c(-n2nc(NS(=O)(=O)c3cccc(N)c3)c3ccc(C(C)(C)C)nc32)c(C)c1] | 1.93 | 463.204 | 464 | A |
| 62 | 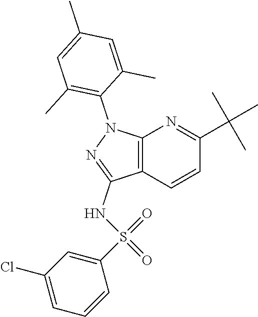 [SMILES: Cc1cc(C)c(-n2nc(NS(=O)(=O)c3cccc(Cl)c3)c3ccc(C(C)(C)C)nc32)c(C)c1] | 2.23 | 482.154 | 483 | A |
| 63 | 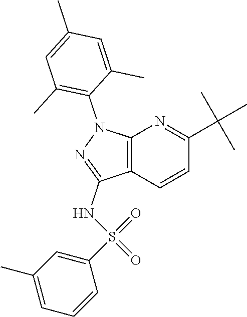 [SMILES: Cc1cccc(S(=O)(=O)Nc2nn(-c3c(C)cc(C)cc3C)c3nc(C(C)(C)C)ccc23)c1] | 2.18 | 462.209 | 463 | A |
| 64 | 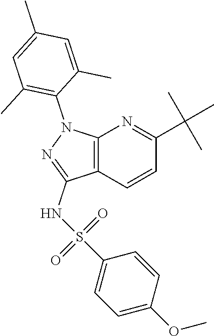 [SMILES: COc1ccc(S(=O)(=O)Nc2nn(-c3c(C)cc(C)cc3C)c3nc(C(C)(C)C)ccc23)cc1] | 2.11 | 478.204 | 479 | A |
| 65 | 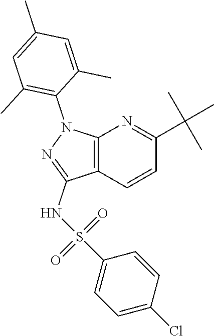 [SMILES: Cc1cc(C)c(-n2nc(NS(=O)(=O)c3ccc(Cl)cc3)c3ccc(C(C)(C)C)nc32)c(C)c1] | 2.23 | 482.154 | 483 | A |
| 66 | 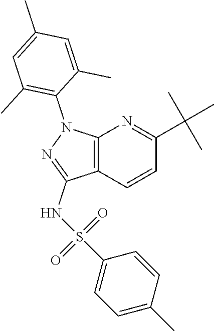 [SMILES: Cc1ccc(S(=O)(=O)Nc2nn(-c3c(C)cc(C)cc3C)c3nc(C(C)(C)C)ccc23)cc1] | 2.17 | 462.209 | 463 | A |
| 67 | 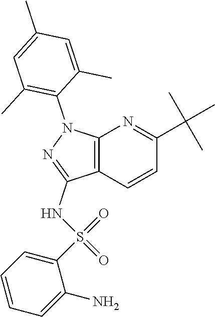 [SMILES: Cc1cc(C)c(-n2nc(NS(=O)(=O)c3ccccc3N)c3ccc(C(C)(C)C)nc32)c(C)c1] | 2.07 | 463.204 | 464 | A |
| 68 | 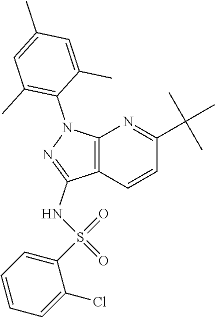 [SMILES: Cc1cc(C)c(-n2nc(NS(=O)(=O)c3ccccc3Cl)c3ccc(C(C)(C)C)nc32)c(C)c1] | 2.16 | 482.154 | 484 | A |
| 69 | 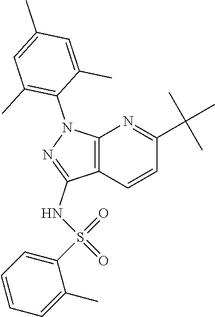 [SMILES: Cc1cc(C)c(-n2nc(NS(=O)(=O)c3ccccc3C)c3ccc(C(C)(C)C)nc32)c(C)c1] | 2.18 | 462.209 | 463 | A |

TABLE 10
NMR data for Compounds 40-69

| Cmpd No. | NMR |
|---|---|
| 44 | ¹H NMR (400 MHZ, DMSO-d6) δ 11.28 (s, 1H), 8.23 (d, J = 8.6 Hz, |
| | 1H), 8.02 (d, J = 8.2 Hz, 2H), 7.90 (d, J = 7.6 Hz, 2H), 7.60 (dt, J = |
| | 15.0, 7.3 Hz, 3H), 7.45 (d, J = 8.6 Hz, 1H), 7.32 (d, J = 8.2 Hz, 2H), |
| | 2.34 (s, 3H), 1.39 (s, 9H). |
| 46 | ¹H NMR (400 MHZ, DMSO) δ 11.23 (s, 1H), 8.60 (d, J = 2.2 Hz, 1H), |
| | 8.02 (d, J = 2.2 Hz, 1H), 7.96-7.85 (m, 2H), 7.72-7.54 (m, 5H), |
| | 6.92 (s, 1H), 3.09 (hept, J = 6.9 Hz, 1H), 2.34 (s, 6H), 1.26 (d, J = 6.9 |
| | Hz, 6H). |
| 47 | ¹H NMR (400 MHZ, DMSO) δ 11.38 (s, 1H), 8.91 (d, J = 2.2 Hz, 1H), |
| | 8.28 (d, J = 2.2 Hz, 1H), 7.98-7.87 (m, 2H), 7.72-7.64 (m, 3H), |
| | 7.64-7.58 (m, 2H), 6.93 (s, 1H), 5.57 (s, 1H), 5.27-5.19 (m, 1H), |
| | 2.35 (s, 7H), 2.18 (s, 3H). |
| 48 | ¹H NMR (400 MHZ, DMSO) δ 11.41 (s, 1H), 8.74 (d, J = 2.3 Hz, 1H), |
| | 8.46 (d, J = 2.3 Hz, 1H), 7.97-7.83 (m, 2H), 7.71-7.64 (m, 1H), |
| | 7.64-7.57 (m, 4H), 6.96 (s, 1H), 2.34 (s, 6H). |
| 49 | ¹H NMR (400 MHZ, DMSO) δ 13.62 (s, 1H), 7.89 (d, J = 8.5 Hz, 1H), |
| | 7.77-7.73 (m, 2H), 7.72-7.65 (m, 1H), 7.63-7.56 (m, 2H), 7.40 |
| | (d, J = 8.6 Hz, 1H), 3.89 (dq, J = 9.4, 6.7 Hz, 1H), 1.39 (s, 9H), 1.34- |
| | 1.25 (m, 1H), 1.04 (d, J = 6.7 Hz, 3H), 0.94 (d, J = 6.5 Hz, 3H), |
| | 0.74 (d, J = 6.7 Hz, 3H). |
| 50 | ¹H NMR (400 MHZ, DMSO) δ 11.38 (s, 1H), 8.27 (d, J = 8.6 Hz, 1H), |
| | 7.97 (t, J = 2.2 Hz, 1H), 7.95-7.92 (m, 1H), 7.91 (d, J = 1.7 Hz, 1H), |
| | 7.74-7.55 (m, 4H), 7.48 (d, J = 8.6 Hz, 1H), 7.41 (t, J = 8.2 Hz, 1H), |
| | 6.82 (dd, J = 8.4, 2.5 Hz, 1H), 3.83 (s, 3H), 1.40 (s, 9H). |
| 51 | ¹H NMR (400 MHZ, DMSO) δ 11.03 (s, 1H), 8.16 (d, J = 8.6 Hz, 1H), |
| | 7.85-7.77 (m, 2H), 7.69-7.52 (m, 3H), 7.50-7.34 (m, 2H), 7.24 |
| | (td, J = 7.3, 6.7, 1.5 Hz, 2H), 7.07 (td, J = 7.6, 1.2 Hz, 1H), 3.67 (s, |
| | 3H), 1.28 (s, 9H) |
| 52 | ¹H NMR (400 MHZ, DMSO) δ 11.09 (s, 1H), 8.47 (d, J = 6.0 Hz, 1H), |
| | 8.32 (d, J = 8.1 Hz, 1H), 7.73 (d, J =7.4 Hz, 2H), 7.60 (t, J = 7.4 Hz, |
| | 1H), 7.51 (t, J = 7.6 Hz, 2H), 7.27 (dd, J = 8.1, 4.5 Hz, 1H), 7.00 (s, |
| | 2H), 2.31 (s, 3H), 2.07 (s, 1H), 1.60 (s, 6H). |
| 53 | ¹H NMR (400 MHZ, DMSO) δ 10.70 (s, 1H), 8.25 (d, J = 8.6 Hz, 1H), |
| | 7.37 (d, J = 8.6 Hz, 1H), 7.16 (d, J = 7.9 Hz, 1H), 7.05 (d, J = 8.1 Hz, |
| | 1H), 6.98 (s, 2H), 6.81 (t, J = 8.0 Hz, 1H), 4.26 (d, J = 19.4 Hz, 4H), |
| | 2.30 (s, 3H), 1.59 (s, 6H), 1.24 (s, 9H). |
| 54 | ¹H NMR (400 MHZ, DMSO) δ 10.38 (s, 1H), 8.30 (d, J = 8.6 Hz, 1H), |
| | 7.41 (t, J = 8.5 Hz, 1H), 7.34 (d, J = 8.6 Hz, 1H), 6.97 (s, 2H), 6.68 |
| | (d, J = 8.5 Hz, 2H), 3.72 (s, 6H), 2.30 (s, 3H), 1.61 (s,6H), 1.24(s, |
| | 9H). |
| 55 | ¹H NMR (400 MHZ, DMSO) δ 10.62 (s, 1H), 8.26 (d, J = 8.6 Hz, 1H), |
| | 7.37 (d, J = 8.6 Hz, 1H), 7.29 (s, 1H), 7.07 (s, 2H), 6.97 (s, 2H), 3.77 |
| | (s, 3H), 2.30 (s, 3H), 1.60 (s, 6H), 1.25 (s, 9H). |
| 56 | ¹H NMR (400 MHZ, DMSO) δ 11.35 (s, 1H), 8.20 (d, J = 8.6 Hz, 1H), |
| | 7.38 (d, J = 8.6 Hz, 1H), 7.17 (s, 2H), 7.00 (s, 2H), 2.31 (s, 3H), 1.70 |
| | (s, 6H), 1.26 (s, 9H). |
| 57 | ¹H NMR (400 MHZ, DMSO) δ 10.94 (s, 1H), 8.30-8.16 (m, 1H), |
| | 7.40 (d, J = 8.3 Hz, 1H), 7.01 (s, 2H), 6.81 (s, 2H), 6.71 (s, 1H), 3.69 |
| | (s, 6H), 2.31 (s, 3H), 1.67 (s, 6H), 1.26 (s, 9H). |
| 58 | ¹H NMR (400 MHZ, DMSO) δ 10.70 (s, 1H), 8.26 (d, J = 8.5 Hz, 1H), |
| | 7.37 (d, J = 8.6 Hz, 1H), 7.13 (d, J = 19.3 Hz, 3H), 6.98 (s, 2H), 3.78 |
| | (s, 3H), 3.63 (s, 3H), 2.30 (s, 3H), 1.59 (s, 6H), 1.24 (s, 9H). |
| 59 | ¹H NMR (400 MHZ, DMSO) δ 10.64 (s, 1H), 8.26 (d, J = 8.5 Hz, 1H), |
| | 7.64 (d, J = 9.1 Hz, 1H), 7.54 (t, J = 7.2 Hz, 1H), 7.36 (d, J = 8.6 Hz, |
| | 1H), 7.16 (d, J = 8.4 Hz, 1H), 7.00-6.92 (m, 3H), 3.84 (s, 3H), 2.29 |
| | (s, 3H), 2.07 (s, 1H), 1.56 (s, 6H), 1.24 (s, 9H). |
| 60 | ¹H NMR (400 MHZ, DMSO) δ 10.97 (s, 1H), 8.21 (d, J = 8.6 Hz, 1H), |
| | 7.45-7.37 (m, 2H), 7.30 (d, J = 6.9 Hz, 1H), 7.22 (s, 1H), 7.17 (d, J = |
| | 10.6 Hz, 1H), 7.00 (s, 2H), 3.70 (s, 3H), 2.31 (s, 3H), 1.65 (s, 6H), |
| | 1.26 (s, 10H). |
| 61 | ¹H NMR (400 MHz, DMSO) δ 10.75 (s, 1H), 8.23 (d, J = 8.5 Hz, 1H), |
| | 7.38 (d, J = 8.5 Hz, 1H), 7.08 (s, 1H), 7.00 (s, 2H), 6.93 (s, 1H), 6.80 |
| | (d, J = 7.6 Hz, 1H), 6.70 (d, J = 8.0 Hz, 1H), 5.54 (s, 1H), 2.31 (s, |
| | 3H), 1.69 (s, 6H), 1.26 (s, 9H). |
| 62 | ¹H NMR (400 MHZ, DMSO) δ 11.17 (s, 1H), 8.22 (d, J = 8.5 Hz, 1H), |
| | 7.71 (d, J = 6.3 Hz, 3H), 7.57 (s, 1H), 7.41 (d, J = 8.5 Hz, 1H), 7.01 |
| | (s, 2H), 2.31 (s, 3H), 1.66 (s, 6H), 1.26 (s, 10H). |
| 63 | ¹H NMR (400 MHZ, DMSO) δ 10.92 (s, 1H), 8.21 (d, J = 8.5 Hz, 1H), |
| | 7.52 (s, 2H), 7.38 (d, J = 8.4 Hz, 3H), 7.00 (s, 2H), 2.29 (d, J = 10.0 |
| | Hz, 6H), 1.66 (s, 6H), 1.26 (s, 9H). |
| 64 | ¹H NMR (400 MHZ, DMSO) δ 10.81 (s, 1H), 8.23 (d, J = 8.6 Hz, 1H), |
| | 7.65 (d, J = 8.9 Hz, 2H), 7.38 (d, J = 8.6 Hz, 1H), 7.08-6.99 (m, |
| | 4H), 3.78 (s, 3H), 2.31 (s, 3H), 1.65 (s, 6H), 1.26 (s, 10H). |
| 65 | ¹H NMR (400 MHZ, DMSO) δ 11.11 (s, 1H), 8.23 (d, J = 8.5 Hz, 1H), |
| | 7.74 (d, J = 8.5 Hz, 2H), 7.61 (d, J = 8.5 Hz, 2H), 7.40 (d, J = 8.5 Hz, |
| | 1H), 7.00 (s, 2H), 2.31 (s, 3H), 1.64 (s, 6H), 1.26 (s, 9H). |
| 66 | ¹H NMR (400 MHZ, DMSO) δ 10.89 (s, 1H), 8.23 (d, J = 8.5 Hz, 1H), |
| | 7.61 (d, J = 8.1 Hz, 2H), 7.38 (d, J = 8.5 Hz, 1H), 7.31 (d, J = 8.0 Hz, |
| | 2H), 6.99 (s, 2H), 2.32 (d, J = 8.4 Hz, 6H), 1.64 (s, 6H), 1.26 (s, 9H). |
| 67 | ¹H NMR (400 MHZ, DMSO) δ 10.79 (s, 1H), 8.22 (d, J = 8.6 Hz, 1H), |
| | 7.35 (dd, J = 11.5, 8.3 Hz, 2H), 7.17 (s, 1H), 6.99 (s, 2H), 6.71 (d, J = |
| | 8.3 Hz, 1H), 6.43 (d, J = 7.3 Hz, 1H), 5.97 (s, 1H), 2.31 (s, 3H), |
| | 2.07 (s, 1H), 1.65 (s, 6H), 1.25 (s, 9H). |
| 68 | ¹H NMR (400 MHZ, DMSO) δ 11.33 (s, 1H), 8.27 (d, J = 8.6 Hz, 1H), |
| | 7.95 (d, J = 8.0 Hz, 1H), 7.61 (s, 2H), 7.48-7.37 (m, 2H), 6.97 (s, |
| | 2H), 2.30 (s, 3H), 1.58 (s, 6H), 1.25 (s, 10H). |

### Compound 70

### N-[6-bromo-1-(o-tolyl)indazol-3-yl]benzenesulfonamide

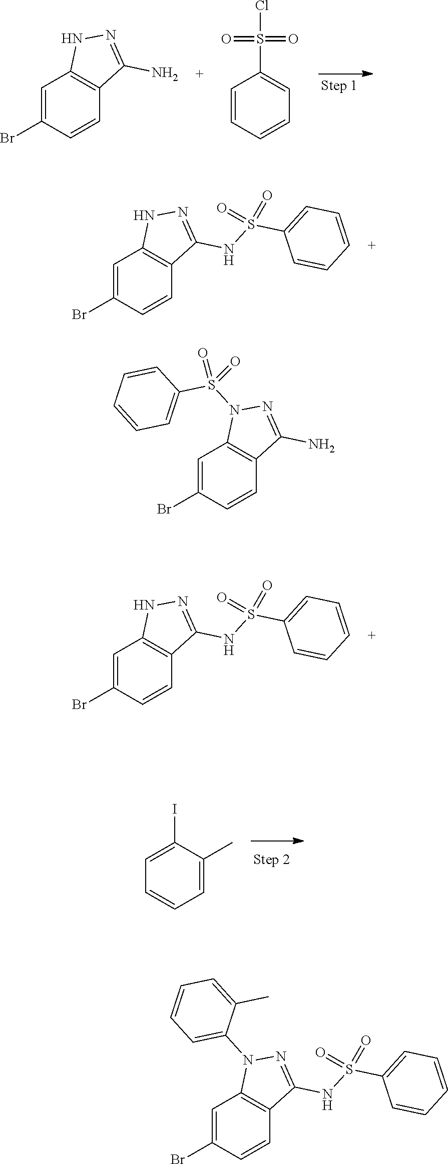

### Step 1: N-(6-bromo-1H-indazol-3-yl)benzenesulfonamide and 1-(benzenesulfonyl)-6-bromo-indazol-3-amine

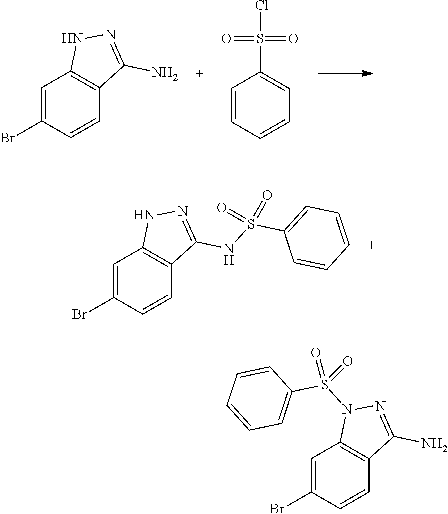 [SMILES: Nc1n[nH]c2cc(Br)ccc12 ; O=S(=O)(Cl)c1ccccc1 ; O=S(=O)(Nc1n[nH]c2cc(Br)ccc12)c1ccccc1 ; Nc1nn(S(=O)(=O)c2ccccc2)c2cc(Br)ccc12]

To a solution of 6-bromo-1H-indazol-3-amine (2.0 g, 9.43 mmol) in pyridine (10 mL) was added benzenesulfonyl chloride (approximately 3.331 g, 2.407 mL, 18.86 mmol) slowly at 0° C. and the reaction was stirred at rt overnight. More benzenesulfonyl chloride (approximately 3.331 g, 2.407 mL, 18.86 mmol) was added and the reaction was stirred overnight at rt again. The reaction mixture was diluted with ethyl acetate, washed with 1 N HCl. The layers were separated, and the organic layer was dried over Na₂SO₄, concentrated, and purified on reverse phase HPLC (HCl modifier, 25-75% ACN-H₂O) to give N-(6-bromo-1H-indazol-3-yl)benzenesulfonamide (1.7 g). ¹H NMR (400 MHz, DMSO) δ 12.73 (s, 1H), 10.73 (s, 1H), 7.76 (d, J=7.7 Hz, 2H), 7.65-7.58 (m, 3H), 7.52 (t, J=7.6 Hz, 2H), 7.19 (d, J=8.5 Hz, 1H). ESI-MS m/z calc. 350.9677, found 353.0 (M+2)⁺; Retention time: 1.36 minutes (LC method A). and 1-(benzenesulfonyl)-6-bromo-indazol-3-amine (325.7 mg). ¹H NMR (400 MHz, DMSO) δ 8.11 (d, J=1.5 Hz, 1H), 7.75 (dd, J=7.9, 1.9 Hz, 3H), 7.67 (t, J=7.5 Hz, 1H), 7.54 (t, J=8.9 Hz, 3H), 6.62 (s, 2H). ESI-MS m/z calc. 350.9677, found 353.0 (M+2)⁺; Retention time: 1.5 minutes (LC method A).

### Step 2: N-[6-bromo-1-(o-tolyl)indazol-3-yl]benzenesulfonamide

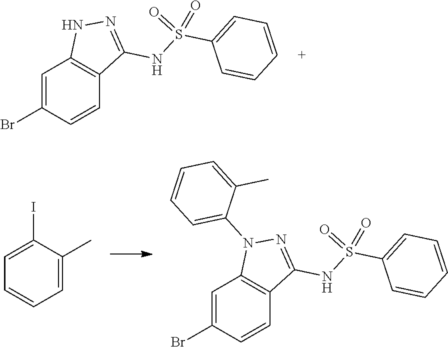

To a mixture of N-(6-bromo-1H-indazol-3-yl)benzenesulfonamide (500 mg, 1.420 mmol), 1-iodo-2-methyl-benzene (200 μL), Cs₂CO₃ (1.142 g, 3.505 mmol), quinolin-8-ol (160 mg, 1.102 mmol), and copper(1+) Iodide (160 mg, 0.8401 mmol) was added t-BuOH (14.68 mL) and the mixture was flushed with nitrogen thoroughly. It was sealed and was heated at 105° C. overnight. The mixture was filtered using a Celite pad using methanol/ethyl acetate (1.1). The filtrate was collected, concentrated and the residue was diluted with DMSO and purified by reverse phase HPLC (HCl modifier, 30-99% ACN-H₂O) to give N-[6-bromo-1-(o-tolyl)indazol-3-yl]benzenesulfonamide (492 mg), ESI-MS m/z calc. 441.01465, found 352.0 (M+1)⁺; Retention time: 1.35 minutes (LC method A).

### Compound 71

The compound in the following Table 11 was prepared in a manner analogous to that described above using commercially available reagents and intermediates described herein.

TABLE 11
Characterization data for Compound 71

| Cmpd | | LCMS | Calc. | | LCMS |
|---|---|---|---|---|---|
| No. | Structure | Rt (min) | mass | M + 1 | Method |
| 71 | 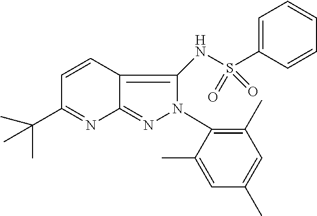 [SMILES: Cc1cc(C)c(-n2nc3nc(C(C)(C)C)ccc3c2NS(=O)(=O)c2ccccc2)c(C)c1] | 1.65 | 448.193 | 449.31 | A |

### Compounds 72 and 73

### N-[6-isopropenyl-1-(o-tolyl)indazol-3-yl]benzenesulfonamide (72) and N-[6-isopropyl-1-(o-tolyl)indazol-3-yl]benzenesulfonamide (73)

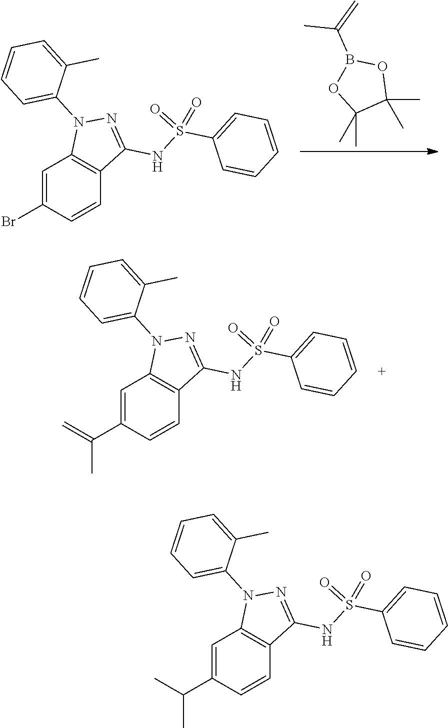 [SMILES: Cc1ccccc1-n1nc(NS(=O)(=O)c2ccccc2)c2ccc(Br)cc21 ; C=C(C)B1OC(C)(C)C(C)(C)O1 ; C=C(C)c1ccc2c(NS(=O)(=O)c3ccccc3)nn(-c3ccccc3C)c2c1 ; Cc1ccccc1-n1nc(NS(=O)(=O)c2ccccc2)c2ccc(C(C)C)cc21]

N-[6-bromo-1-(o-tolyl)indazol-3-yl]benzenesulfonamide (45 mg, 0.1017 mmol), Pd(dppf)Cl₂ (approximately 3.721 mg, 0.005085 mmol), sodium carbonate (200 μL of 2 M, 0.4000 mmol), and 2-isopropenyl-4,4,5,5-tetramethyl-1,3,2-dioxaborolane (approximately 17.09 mg, 0.1017 mmol) in dioxane (1 mL) were added to a microwave vial. The vial was purged with nitrogen, capped and heated at 120° C. for 45 min in the microwave. The crude was purified by HPLC utilizing a gradient of 30-90% acetonitrile in 5 mM aqueous HCl to give N-[6-isopropenyl-1-(o-tolyl)indazol-3-yl]benzenesulfonamide (8.8 mg)¹H NMR (400 MHz, DMSO) δ 10.92 (s, 1H), 7.79 (dd, J=8.2, 5.0 Hz, 3H), 7.62 (t, J=7.0 Hz, 1H), 7.54 (t, J=7.7 Hz, 2H), 7.45-7.40 (m, 3H), 7.39-7.31 (m, 2H), 7.05 (s, 1H), 5.47 (s, 1H), 5.16 (s, 1H), 2.08 (s, 3H), 1.80 (s, 3H). ESI-MS m/z calc. 403.13544, found 403.0 (M+1)⁺; Retention time: 1.99 minutes (LC method A).

The product was dissolved in MeOH (25 mL) and Pd/C (20 mg of 5% w/w, 0.009397 mmol) was added. The mixture was stirred under hydrogen balloon at rt overnight. The reaction mixture was filtered, concentrated, and purified on reverse phase HPLC (HCl modifier, 30-99% ACN-H₂O) to give N-[6-isopropyl-1-(o-tolyl)indazol-3-yl]benzenesulfonamide (4.4 mg), ESI-MS m/z calc. 405.1511, found 406.0 (M+1)⁺; Retention time: 2.04 minutes (LC method A).

### Compounds 74 and 75

The compounds in the following Table 12 were prepared in a manner analogous to that described above using commercially available reagents and intermediates described herein.

TABLE 12
Characterization data for Compounds 74 and 75

| | | LCMS | | | | |
|---|---|---|---|---|---|---|
| Cmpd | | Rt | Calc. | | LCMS | |
| No. | Structure | (min) | mass | M + 1 | Met. | NMR |
| 74 | 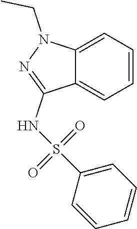 [SMILES: CCn1nc(NS(=O)(=O)c2ccccc2)c2ccccc21] | 1.37 | 301.088 | 302 | A | ¹H NMR (400 MHz, DMSO) δ 10.58 (s, 1H), 7.75 (d, J = 7.8 Hz, 2H), 7.66-7.49 (m, 5H), 7.40-7.31 (m, 1H), 7.14-7.03 (m, 1H), 4.25 (q, J = 7.1 Hz, 2H), 1.23 (t, J = 7.1 Hz, 3H) |
| 75 | 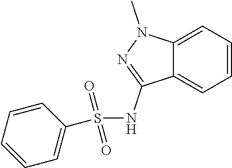 [SMILES: Cn1nc(NS(=O)(=O)c2ccccc2)c2ccccc21] | 1.2 | 287.073 | 288 | A | ¹H NMR (400 MHz, DMSO) δ 10.62 (s, 1H), 7.78 (d, J = 7.6 Hz, 2H), 7.66-7.50 (m, 5H), 7.41-7.32 (m, 1H), 7.16-7.02 (m, 1H), 3.87 (s, 3H) |

### Compound 76

### N-[4-(2,6-Dimethylphenyl)-8-methyl-8-phenyl-6,7-dihydro-5H-quinazolin-2-yl]benzenesulfonamide

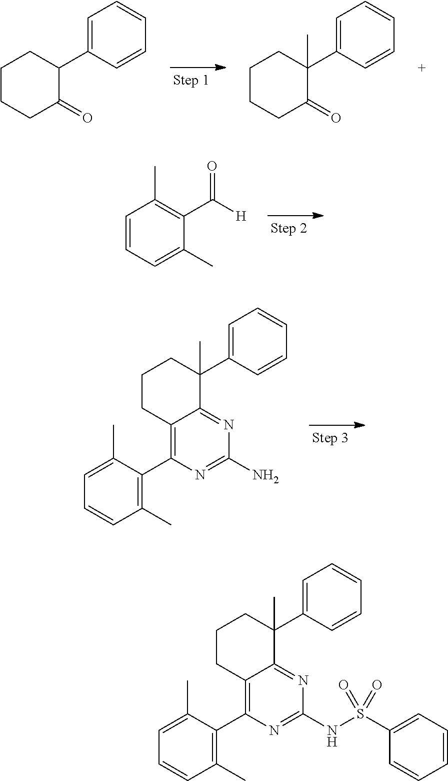

### Step 1: 2-Methyl-2-phenyl-cyclohexanone

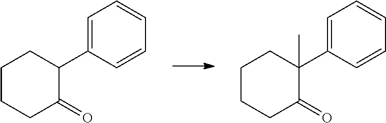

In a 100 mL round-bottomed flask, 2-phenylcyclohexanone (830.5 mg, 4.766 mmol) was dissolved in dimethylformamide (16 mL) and this solution was cooled to 0° C.; 60% NaH (250.9 mg, 6.273 mmol) was added, and this slurry was stirred at 0° C. for 10 min. Then, methyl iodide (400 μL, 6.425 mmol) was added, and the reaction mixture was stirred at 0° C. for 50 min. The reaction mixture was quenched with 1 N HCl (20 mL) and extracted with ethyl acetate (3×40 mL). The combined organic extracts were washed with water (50 mL) and saturated aqueous sodium chloride solution (50 mL), then dried over sodium sulfate, filtered, and evaporated in vacuo. This crude product was purified by silica gel chromatography (80 g of silica, 0 to 10% gradient of ethyl acetate/hexanes) to give a slightly yellow liquid, 2-methyl-2-phenyl-cyclohexanone (631.2 mg, 70%). ESI-MS m/z calc. 188.12012, found 189.2 (M+1)⁺; Retention time: 1.61 minutes; LC method A. ¹H NMR (400 MHz, chloroform-d) δ 7.39-7.31 (m, 2H), 7.28-7.16 (m, 3H), 2.74-2.63 (m, 1H), 2.44-2.26 (m, 2H), 2.00-1.91 (m, 1H), 1.80-1.66 (m, 4H), 1.27 (s, 3H).

### Step 2: 4-(2,6-Dimethylphenyl)-8-methyl-8-phenyl-6,7-dihydro-5H-quinazolin-2-amine

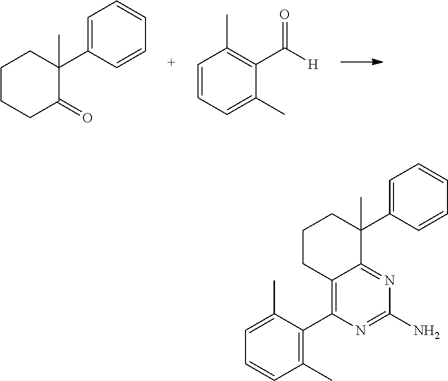

Stage 1: To a 20 mL vial with a pressure-relief cap, 2-methyl-2-phenyl-cyclohexanone (405.1 mg, 2.152 mmol), 2,6-dimethylbenzaldehyde (260.4 mg, 1.941 mmol), potassium carbonate (409.6 mg, 2.964 mmol) and ethanol (6.0 mL) were added, and this slurry was stirred at 70° C. for 14 h then at 90° C. for 48 h. The reaction mixture was then cooled to room temperature, filtered, and purified by reverse phase HPLC (1-99% acetonitrile in water using HCl as a modifier) to give (6E)-6-[(2,6-dimethylphenyl)methylene]-2-methyl-2-phenyl-cyclohexanone (101.3 mg, 17%) ESI-MS m/z calc. 304.1827, found 305.3 (M+1)⁺; Retention time: 2.28 minutes.

Stage 2: In a 20 mL vial, the product from Stage 1, guanidine (Carbonic Acid (0.5)) (175.1 mg, 1.944 mmol) and potassium carbonate (270.3 mg, 1.956 mmol) were mixed together with N-methylpyrrolidinone (4.0 mL), and stirred at 170° C. for 22 h. This mixture was cooled to room temperature, upon which 1,4-cyclohexadiene (406.3 mg, 5.071 mmol) was added. This mixture was stirred at 150° C. for 4 h, then cooled to room temperature, and quenched with 1 N HCl (6 mL). The mixture was extracted with ethyl acetate (3×8 mL). The combined organic extracts was washed with water (15 mL) and saturated aqueous sodium chloride solution (15 mL), then dried over sodium sulfate, filtered, and evaporated in vacuo to give 4-(2,6-dimethylphenyl)-8-methyl-8-phenyl-6,7-dihydro-5H-quinazolin-2-amine (99.8 mg, 15%). ¹H NMR (400 MHz, dimethylsulfoxide-d6) δ 7.38-7.25 (m, 3H), 7.25-7.17 (m, 3H), 7.16-7.08 (m, 2H), 3.85-3.15 (bs, 2H), 2.16-1.98 (m, 3H), 2.09 (s, 3H), 2.06 (s, 3H), 1.93-1.84 (m, 1H), 1.78 (s, 3H), 1.64-1.51 (m, 1H), 1.47-1.31 (m, 1H) ESI-MS m/z calc. 343.20483, found 344.5 (M+1)⁺; Retention time: 1.56 minutes (LC method A).

### Step 3: N-[4-(2,6-Dimethylphenyl)-8-methyl-8-phenyl-6,7-dihydro-5H-quinazolin-2-yl]benzenesulfonamide

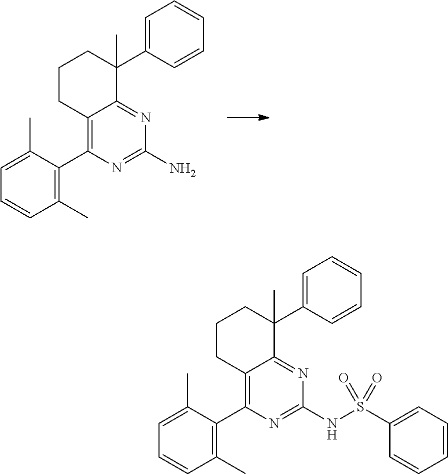 [SMILES: Cc1cccc(C)c1-c1nc(N)nc2c1CCCC2(C)c1ccccc1 ; Cc1cccc(C)c1-c1nc(NS(=O)(=O)c2ccccc2)nc2c1CCCC2(C)c1ccccc1]

In a 3 mL vial, 4-(2,6-dimethylphenyl)-8-methyl-8-phenyl-6,7-dihydro-5H-quinazolin-2-amine (38.5 mg, 0.112 mmol) was dissolved in MeCN (900 μL), to which DABCO (79.8 mg, 0.711 mmol) and PhSO₂Cl (90 μL, 0.71 mmol) were added. This mixture was stirred at 90° C. for 61 h, after which it was cooled to room temperature, filtered, and purified by reverse phase HPLC (1-99% acetonitrile in water using HCl as a modifier) to give 1.6 mg of slightly impure product. This material was further purified by preparative TLC (one full silica plate, 20 cm×20 cm, 250 μm thickness, 60 Å particle size, 10% ethyl acetate/hexanes elution, UV active band) to give N-[4-(2,6-dimethylphenyl)-8-methyl-8-phenyl-6,7-dihydro-5H-quinazolin-2-yl]benzenesulfonamide (1.2 mg, 2%). ESI-MS m/z calc. 483.19806, found 484.4 (M+1)⁺; Retention time: 2.21 minutes; LC method A.

### Compound 77

### N-[1-(3,5-dimethylphenyl)-3-isoquinolyl]benzenesulfonamide

### Step 1: 3-Chloro-1-(3,5-dimethylphenyl)isoquinoline

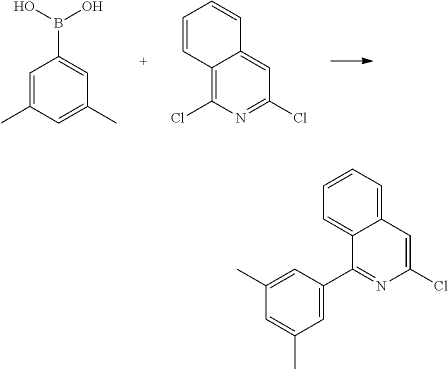

A pressure tube was charged with 1,3-dichloroisoquinoline (200 mg, 1.010 mmol), (3,5-dimethylphenyl)boronic acid (200 mg, 1.333 mmol), Pd(PPh₃)₄ (47 mg, 0.04067 mmol), CsF (338 mg, 2.225 mmol), and 1,2-dimethoxyethane (1.5 mL). The mixture was degassed by flow of nitrogen and heated at 80° C. for 19 h. EtOAc and water were added to the reaction and the two layers were separated. The organic layer was washed with brine (×2), dried over Na₂SO₄, filtered through a plug of Celite and concentrated. The crude product was purified on 24 g of silica gel utilizing a gradient of 0-15% ethyl acetate in hexane to yield 3-chloro-1-(3,5-dimethylphenyl)isoquinoline (230 mg, 85%) as a colorless viscous liquid. ESI-MS m/z calc. 267.08148, found 268.1 (M+1)⁺; Retention time: 2.17 minutes (LC method A).

### Step 2: N-[1-(3,5-dimethylphenyl)-3-isoquinolyl]benzenesulfonamide

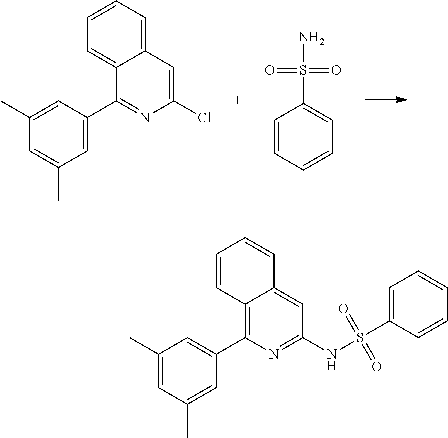 [SMILES: Cc1cc(C)cc(-c2nc(Cl)cc3ccccc23)c1 ; NS(=O)(=O)c1ccccc1 ; Cc1cc(C)cc(-c2nc(NS(=O)(=O)c3ccccc3)cc3ccccc23)c1]

Nitrogen was bubbled through a mixture of 3-chloro-1-(3,5-dimethylphenyl)isoquinoline (50 mg, 0.1867 mmol), benzenesulfonamide (60 mg, 0.3817 mmol), (5-diphenylphosphanyl-9,9-dimethyl-xanthen-4-yl)-diphenyl-phosphane (17 mg, 0.02938 mmol), palladium diacetate (10 mg, 0.04454 mmol) and cesium carbonate (123 mg, 0.3775 mmol) in 1,4-dioxane (1.4 mL) for 5 min at rt. The reaction was stirred at rt for 17 h. The reaction was heated at 130° C. for 5 h. The crude product was filtered and purified using a reverse phase HPLC C₁₈ column and a dual gradient run from 1-99% mobile phase B over 30 minutes [(Mobile phase A=H₂O (5 mM HCl), Mobile phase B=CH₃CN)] to yield N-[1-(3,5-dimethylphenyl)-3-isoquinolyl]benzenesulfonamide (16.6 mg, 23%) as a yellow solid. ¹H NMR (400 MHz, DMSO-d₆) δ 11.10 (s, 1H), 7.97-7.86 (m, 4H), 7.70-7.60 (m, 2H), 7.58-7.52 (m, 2H), 7.50-7.41 (m, 2H), 7.15 (s, 1H), 7.04 (s, 2H), 2.35 (s, 6H). ESI-MS m/z calc. 388.12454, found 389.2 (M+1)⁺; Retention time: 2.03 minutes (LC method A).

### Compound 78

### N-[4-(3,5-Dimethylphenyl)-2-quinolyl]benzenesulfonamide

### Step 1: N-(4-chloro-2-quinolyl)benzenesulfonamide

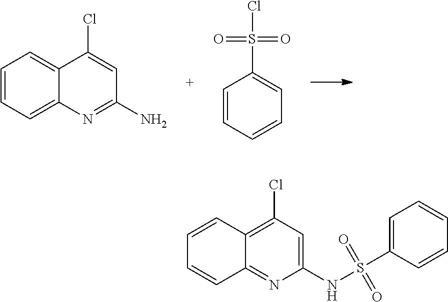 [SMILES: Nc1cc(Cl)c2ccccc2n1 ; O=S(=O)(Cl)c1ccccc1 ; O=S(=O)(Nc1cc(Cl)c2ccccc2n1)c1ccccc1]

To a pressure vessel containing a solution of 4-chloroquinolin-2-amine (200 mg, 1.120 mmol) in pyridine (2.9 mL) was added benzenesulfonyl chloride (145 μL, 1.136 mmol) and the reaction was stirred at 200° C. for 15 min. EtOAc was added to the reaction and washed with water (×3). The organic layer was dried over Na₂SO₄, filtered and concentrated to yield N-(4-chloro-2-quinolyl)benzenesulfonamide (210 mg, 59%) as a brown solid. The product was used in the next step without further purification. ESI-MS m/z calc. 318.02298, found 319.1 (M+1)⁺; Retention time: 0.58 minutes; LC method D.

### Step 2: N-[4-(3,5-Dimethylphenyl)-2-quinolyl]benzenesulfonamide

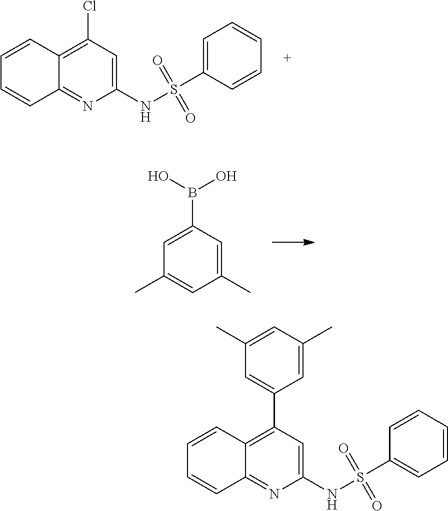

The mixture of N-(4-chloro-2-quinolyl)benzenesulfonamide (100 mg, 0.3137 mmol), (3,5-dimethylphenyl)boronic acid (66 mg, 0.4400 mmol), Pd(dppf)Cl₂ (30 mg, 0.04100 mmol), dioxane (2.000 mL) and potassium carbonate (315 μL of 2 M, 0.6300 mmol) was degassed by flow of nitrogen and stirred at 200° C. for 15 min in a pressure vessel. The cooled mixture was filtered and diluted with EtOAc and washed with water (×1). The aqueous layer was extracted with EtOAc (×3). The combined organic layer was dried over Na₂SO₄, filtered and concentrated in vacuo. The crude product was dissolved in DMSO, filtered and purified using a reverse phase HPLC C₁₈ column and a dual gradient run from 1-99% mobile phase B over 30 minutes [(Mobile phase A=H₂O (5 mM HCl). Mobile phase B=CH₃CN)] to yield N-[4-(3,5-dimethylphenyl)-2-quinolyl]benzenesulfonamide (65.7 mg, 52%). ¹H NMR (400 MHz, DMSO-d6) δ 13.36 (s, 1H), 7.86 (d, J=7.1 Hz, 2H), 7.77-7.67 (m, 1H), 7.67-7.62 (m, 1H), 7.61-7.52 (m, 4H), 7.45-7.31 (m, 2H), 7.21 (s, 1H), 7.00 (s, 2H), 2.37 (s, 6H). ESI-MS m/z calc. 388.12454, found 389.2 (M+1)⁺; Retention time: 1.86 minutes, LC method A.

### Compound 79

### 1-Methyl-N-[1-(o-tolyl)benzofuro[3,2-c]pyridin-3-yl]pyrazole-4-sulfonamide

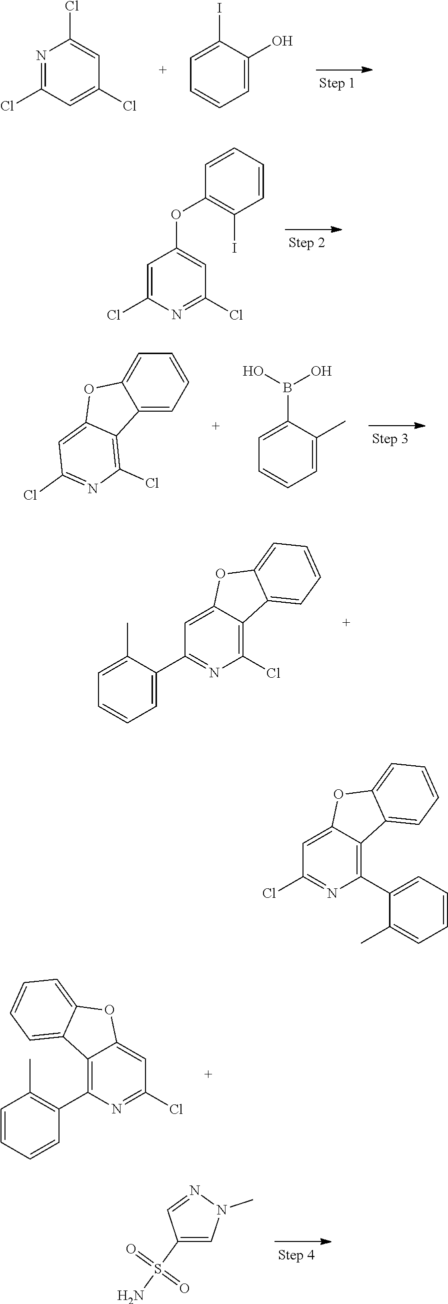

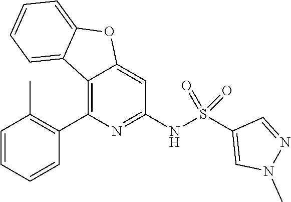 [SMILES: Cc1ccccc1-c1nc(NS(=O)(=O)c2cnn(C)c2)cc2oc3ccccc3c12]

### Step 1: 2,6-Dichloro-4-(2-iodophenoxy)pyridine

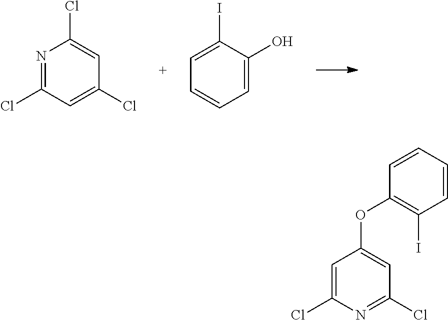 [SMILES: Clc1cc(Cl)nc(Cl)c1 ; Oc1ccccc1I ; Clc1cc(Oc2ccccc2I)cc(Cl)n1]

A heterogeneous solution of 2,4,6-trichloropyridine (1 g, 5.481 mmol), 2-iodophenol (1.2 g, 5.454 mmol), and cesium carbonate (3.6 g, 11.05 mmol) in NMP (11 mL) was stirred at 23° C. for 16 h. The reaction mixture was partitioned between ethyl acetate and water. The organic layer was separated, and the aqueous layer was further extracted with ethyl acetate (2×). The combined organics were washed with brine, dried over magnesium sulfate, filtered, and concentrated in vacuo. The crude residue was separated by flash column chromatography on silica gel (gradient: 1 to 3% ethyl acetate in hexanes) to afford 2,6-dichloro-4-(2-iodophenoxy)pyridine (1.05 g, 52%) as a white crystalline solid. ¹H NMR (400 MHz, Chloroform-d) δ 7.93 (dd, J=7.9, 1.5 Hz, 1H), 7.45 (ddd, J=8.1, 7.4, 1.5 Hz, 1H), 7.16-7.04 (m, 2H), 6.73 (s, 2H). ESI-MS m/z calc. 364.88712, found 365.9 (M+1)⁺; Retention time: 0.77 minutes (LC method D).

### Step 2: 1,3-Dichlorobenzofuro[3,2-c]pyridine

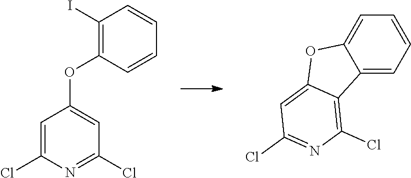 [SMILES: Clc1cc(Oc2ccccc2I)cc(Cl)n1 ; Clc1cc2oc3ccccc3c2c(Cl)n1]

A heterogeneous solution consisting of 2,6-dichloro-4-(2-iodophenoxy)pyridine (1200 mg, 3.279 mmol), diacetoxypalladium (36.8 mg, 0.1639 mmol), 1,3-bis(2,6-diisopropylphenyl)imidazol-1-ium chloride (140 mg, 0.3294 mmol), and potassium carbonate (906 mg, 6.555 mmol) in DME (13 mL) was heated to 130° C. in a sealed vial for 16 h. The reaction mixture was concentrated onto silica gel and separated by flash column chromatography (gradient: 1 to 3% ethyl acetate in hexanes) to afford 1,3-dichlorobenzofuro[3,2-c]pyridine (70 mg, 9%) as a white solid. ¹H NMR (400 MHz, Chloroform-d) δ 8.30-8.23 (m, 1H), 7.65-7.56 (m, 2H), 7.54-7.46 (m, 2H). ESI-MS m/z calc. 236.97482, found 237.94 (M+1)⁺; Retention time: 0.72 minutes, LC method D.

### Step 3: 1-Chloro-3-(o-tolyl)benzofuro[3,2-c]pyridine and 3-chloro-1-(o-tolyl)benzofuro[3,2-c]pyridine

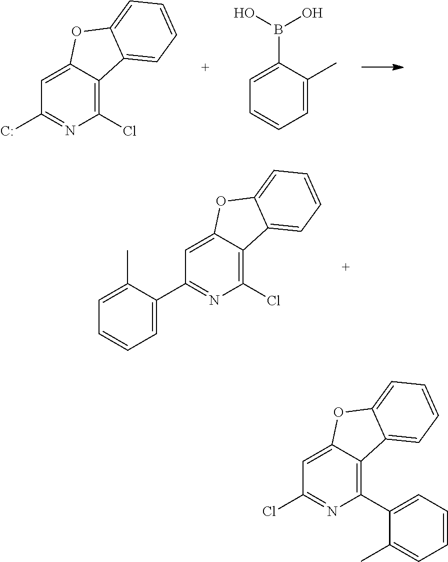

A heterogeneous solution of 1,3-dichlorobenzofuro[3,2-c]pyridine (67.8 mg, 0.2706 mmol), o-tolylboronic acid (36.8 mg, 0.2707 mmol), tetrakis(triphenylphosphine)palladium(0) (62.5 mg, 0.05409 mmol), and potassium carbonate (112 mg, 0.8104 mmol) in dioxane (1.4 mL) was microwaved to 85° C. for 75 min. The organic layer was removed under a steady stream of air from a pipette. The crude residue was redissolved in DCM and concentrated in vacuo onto silica gel. The crude impregnated silica was separated by flash column chromatography (10 to 100% ethyl acetate in hexanes) to afford both 1-chloro-3-(o-tolyl)benzofuro[3,2-c]pyridine (31 mg, 16%) (peak 2) ESI-MS m/z calc. 293.06073, found 294.0 (M+1)⁺; Retention time: 0.83 minutes and 3-chloro-1-(o-tolyl)benzofuro[3,2-c]pyridine (12 mg, 14%) (peak 1) ESI-MS m/z calc. 293.06073, found 293.9 (M+1)⁺; Retention time: 0.87 minutes, LC method D.

### Step 4: 1-Methyl-N-[1-(o-tolyl)benzofuro[3,2-c]pyridin-3-yl]pyrazole-4-sulfonamide

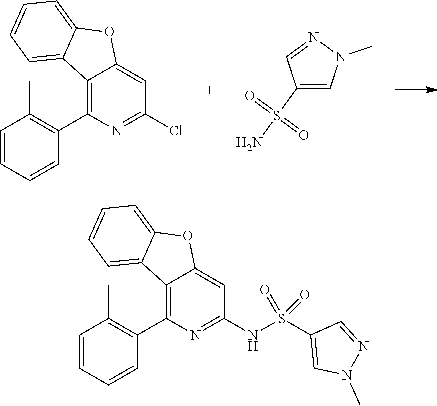

A heterogeneous solution of 3-chloro-1-(o-tolyl)benzofuro[3,2-c]pyridine (9.7 mg, 0.03302 mmol), 1-methylpyrazole-4-sulfonamide (16.5 mg, 0.1024 mmol), potassium carbonate (14 mg, 0.1013 mmol), xantphos (7.8 mg, 0.01348 mmol), and palladium acetate (1.5 mg, 0.006681 mmol) in dioxane (500 μL) was microwaved to 125° C. for 25 min. The reaction mixture was acidified with acetic acid (15 μL, 0.2638 mmol). The sample was purified by reverse phase HPLC (Phenomenex Luna C₁₈ column (75×30 mm, 5 μm particle size), gradient: 1-99% acetonitrile in water (5 mM HCl) over 15.0 minutes) to afford 1-methyl-N-[1-(o-tolyl)benzofuro[3,2-c]pyridin-3-yl]pyrazole-4-sulfonamide (8.8 mg, 64%) as a white solid. ESI-MS m/z calc. 418.10995, found 419.0 (M+1)⁺; Retention time: 1.72 minutes (LC method A). ¹H NMR (400 MHz, Chloroform-d) δ 7.84 (s, 1H), 7.76 (d, J=0.7 Hz, 1H), 7.61-7.52 (m, 2H), 7.48-7.31 (m, 5H), 7.16 (td, J=7.6, 1.0 Hz, 1H), 7.03 (ddd, J=7.8, 1.4, 0.6 Hz, 1H), 3.86 (s, 3H), 2.12 (d, J=0.7 Hz, 3H).

### Compounds 80-82

The compounds in the following Tables 13-15 were prepared in a manner analogous to that described above using commercially available reagents and intermediates described herein.

TABLE 13
Characterization data for Compounds 80-82

| Cmpd | | LCMS | | | LCMS |
|---|---|---|---|---|---|
| No. | Structure | Rt (min) | Calc. mass | M + 1 | Method |
| 80 | 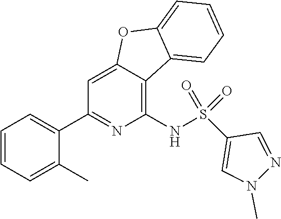 [SMILES: Cc1ccccc1-c1cc2oc3ccccc3c2c(NS(=O)(=O)c2cnn(C)c2)n1] | 1.74 | 418.11 | 419 | A |
| 81 | 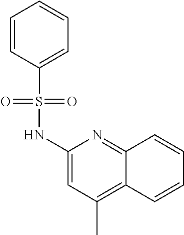 [SMILES: Cc1cc(NS(=O)(=O)c2ccccc2)nc2ccccc12] | 1.25 | 298.078 | 299 | A |
| 82 | 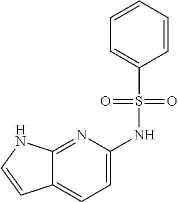 [SMILES: O=S(=O)(Nc1ccc2cc[nH]c2n1)c1ccccc1] | 1.1 | 273.057 | 275 | A |

TABLE 14
NMR data for Compounds 80 and 81

| Cmpd No. | NMR |
|---|---|
| 80 | ¹H NMR (400 MHZ, Chloroform-d) δ 12.16 (s, 1H), |
| | 8.37-8.29 (m, 1H), 7.89-7.79 (m, 2H), 7.60 (dt, |
| | J = 8.2, 0.8 Hz, 1H), 7.54-7.30 (m, 6H), 6.92 (s, 1H), |
| | 3.90 (s, 3H), 2.45 (s, 3H). |
| 81 | ¹H NMR (400 MHz, DMSO) δ 13.09 (s, 1H), 7.90 |
| | (d, J = 7.7 Hz, 3H), 7.71-7.66 (m, 1H), 7.56 |
| | (t, J = 9.6 Hz, 4H), 7.48-7.38 (m, 2H), 2.58 (s, 3H). |

TABLE 15
Characterization data for Compounds 83-95

| Cmpd | | LCMS | Calc. | | LCMS |
|---|---|---|---|---|---|
| No. | Molecule | Rt (min) | Mass | M + 1 | Method |
| 83 | 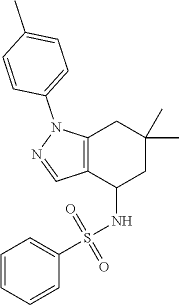 [SMILES: Cc1ccc(-n2ncc3c2CC(C)(C)CC3NS(=O)(=O)c2ccccc2)cc1] | 1.84 | 395.167 | 396 | A |
| 84 | 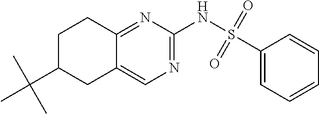 [SMILES: CC(C)(C)C1CCc2nc(NS(=O)(=O)c3ccccc3)ncc2C1] | 1.75 | 345.151 | 346 | A |
| 85 | 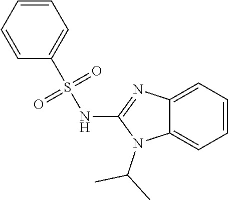 [SMILES: CC(C)n1c(NS(=O)(=O)c2ccccc2)nc2ccccc21] | 1.46 | 315.104 | 316 | A |
| 86 | 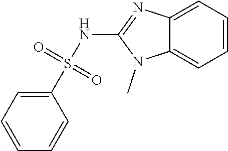 [SMILES: Cn1c(NS(=O)(=O)c2ccccc2)nc2ccccc21] | 1.19 | 287.073 | 288 | A |
| 87 | 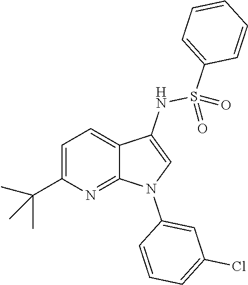 [SMILES: CC(C)(C)c1ccc2c(NS(=O)(=O)c3ccccc3)cn(-c3cccc(Cl)c3)c2n1] | 2.36 | 439.112 | 440.31 | A |
| 88 | 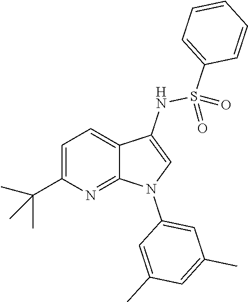 [SMILES: Cc1cc(C)cc(-n2cc(NS(=O)(=O)c3ccccc3)c3ccc(C(C)(C)C)nc32)c1] | 2.4 | 433.182 | 434.36 | A |
| 89 | 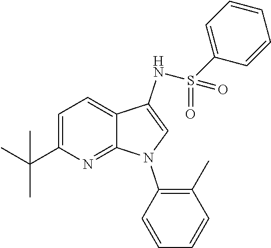 [SMILES: Cc1ccccc1-n1cc(NS(=O)(=O)c2ccccc2)c2ccc(C(C)(C)C)nc21] | 2.19 | 419.167 | 420.39 | A |
| 90 | 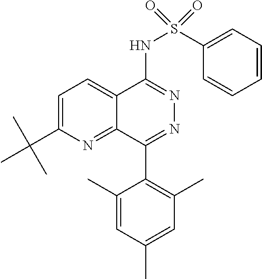 [SMILES: Cc1cc(C)c(-c2nnc(NS(=O)(=O)c3ccccc3)c3ccc(C(C)(C)C)nc23)c(C)c1] | 2.33 | 460.193 | 461.5 | A |
| 91 | 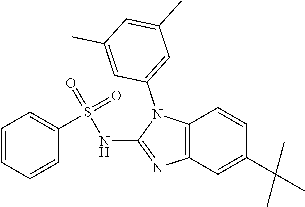 [SMILES: Cc1cc(C)cc(-n2c(NS(=O)(=O)c3ccccc3)nc3cc(C(C)(C)C)ccc32)c1] | 3.15 | 433.182 | 434.4 | I |
| 92 | 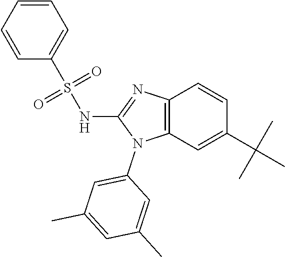 [SMILES: Cc1cc(C)cc(-n2c(NS(=O)(=O)c3ccccc3)nc3ccc(C(C)(C)C)cc32)c1] | 3.21 | 433.182 | 434.4 | I |
| 93 | 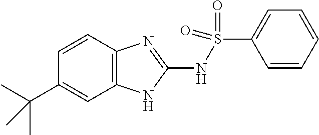 [SMILES: CC(C)(C)c1ccc2nc(NS(=O)(=O)c3ccccc3)[nH]c2c1] | 1.57 | 329.12 | 330.2 | A |
| 94 | 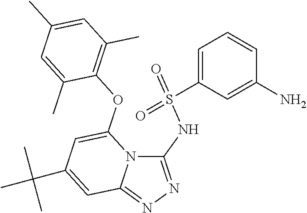 [SMILES: Cc1cc(C)c(Oc2cc(C(C)(C)C)cc3nnc(NS(=O)(=O)c4cccc(N)c4)n23)c(C)c1] | 1.72 | 479.199 | 480 | A |
| 95 | 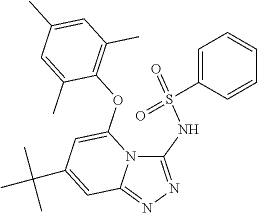 [SMILES: Cc1cc(C)c(Oc2cc(C(C)(C)C)cc3nnc(NS(=O)(=O)c4ccccc4)n23)c(C)c1] | 2.04 | 464.188 | 465 | A |

TABLE 16
NMR data for Compounds 83, 90-94

| Cmpd No. | NMR |
|---|---|
| 83 | ¹H NMR (400 MHZ, DMSO) δ 8.03 (d, J = 8.3 Hz, 1H), 7.92 (d, J = |
| | 7.0 Hz, 2H), 7.74-7.60 (m, 3H), 7.33 (d, J = 8.4 Hz, 2H), 7.27 (d, J = |
| | 8.4 Hz, 2H), 6.86 (s, 1H), 4.24 (s, 1H), 2.60 (d, J = 16.2 Hz, 1H), 2.35- |
| | 2.25 (m, 4H), 1.53-1.35 (m, 2H), 0.99 (s, 3H), 0.76 (s, 3H). |
| 86 | ¹H NMR (400 MHZ, DMSO) δ 10.95 (s, 1H), 9.38 (s, 1H), 7.90 (d, J = |
| | 7.3 Hz, 2H), 7.84 (d, J = 8.6 Hz, 2H), 7.58 (t, J = 7.9 Hz, 1H), 7.54- |
| | 7.45 (m, 5H), 6.47 (t, J = 7.5 Hz, 2H). |
| 90 | ¹H NMR (400 MHZ, DMSO-d6) δ 13.26 (s, 1H), 8.68 (d, J = 8.6 Hz, |
| | 1H), 8.07 (d, J = 7.1 Hz, 2H), 7.99 (d, J = 8.6 Hz, 1H), 7.70-7.54 (m, |
| | 3H), 6.98 (s, 2H), 2.32 (s, 3H), 1.92 (s, 6H), 1.19 (s, 9H). |
| 91 | ¹H NMR (400 MHZ, DMSO) δ 11.98 (s, 1H), 7.88-7.81 (m, 2H), 7.59- |
| | 7.48 (m, 3H), 7.43 (d, J = 8.4 Hz, 1H), 7.31 (dd, J = 8.5, 1.7 Hz, 1H), |
| | 7.17 (s, 1H), 7.03-6.97 (m, 2H), 6.85 (d, J = 1.7 Hz, 1H), 2.33 (s, 6H), |
| | 1.22 (s, 9H). |
| 92 | ¹H NMR (400 MHZ, DMSO) δ 11.93 (s, 1H), 7.88-7.81 (m, 2H), 7.58- |
| | 7.50 (m, 4H), 7.22 (dd, J = 8.5, 1.8 Hz, 1H), 7.15 (s, 1H), 7.02-6.98 |
| | (m, 2H), 6.90 (d, J = 8.5 Hz, 1H), 2.32 (s, 6H), 1.30 (s, 9H). |
| 93 | ¹H NMR (400 MHZ, DMSO) δ 11.82 (s, 2H), 7.91-7.85 (m, 2H), 7.54- |
| | 7.47 (m, 3H), 7.28-7.26 (m, 1H), 7.19 (d, J = 1.2 Hz, 2H), 1.28 (s, |
| | 9H). |

### V. Bioactivity Assays

### A. 3T3 assay

1. Membrane Potential Optical Methods for Assaying F508del Modulation Properties of Compounds

The assay utilizes fluorescent voltage sensing dyes to measure changes in membrane potential using a fluorescent plate reader (e.g., FLIPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.

2. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NIH 3T3 cells. The F508del NIH 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM Ivacaftor.

3. Solutions

Bath Solution #1: (in mM) NaCl 160, KCl 4.5, CaCl₂) 2, MgCl₂ 1, HEPES 10, pH 7.4 with NaOH.

Chloride-free bath solution: Chloride salts in Bath Solution #1 (above) are substituted with gluconate salts.

4. Cell Culture

NIH3T3 mouse fibroblasts stably expressing F508del are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, b-ME, 1×pen/strep, and 25 mM HEPES in 175 cm² culture flasks. For all optical assays, the cells were seeded at ˜20,000/well in 384-well Matrigel-coated plates. For the correction assays, the cells are cultured at 37° C. with and without compounds for 16-24 hours.

### B. Enteroid Assay

1. Solutions

Base medium (ADF+++) consisted of Advanced DMEM/Ham's F12, 2 mM Glutamax, 10 mM HEPES, 1 μg/mL penicillin/streptomycin.

Intestinal enteroid maintenance medium (IEMM) consisted of ADF+++, 1×B27 supplement, 1×N2 supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath1 Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45).

3. Enteroid Cell Harvesting and Seeding

Cells were recovered in cell recovery solution, collected by centrifugation at 650 rpm for 5 min at 4° C., resuspended in TrypLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM (S)—N-((6-aminopyridin-2-yl)sulfonyl)-6-(3-fluoro-5-isobutoxyphenyl)-2-(2,2,4-trimethylpyrrolidin-1-yl)nicotinamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

Table 17 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” 0% Activity: +++ is >60%; ++ is 30-60%; + is <300%).

TABLE 17
CTFR Modulating Activity of Compounds

| | | | 3T3 | | Ent. |
|---|---|---|---|---|---|
| | | 3T3 | Max | Ent. | Max |
| | | EC50 | Activity | EC50 | Activity |
| Cmpd No. | Structure | (μM) | (%) | (μM) | (%) |
| 1 | 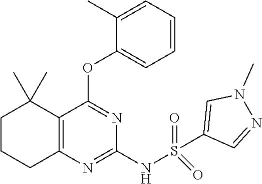 [SMILES: Cc1ccccc1Oc1nc(NS(=O)(=O)c2cnn(C)c2)nc2c1C(C)(C)CCC2] | ++ | +++ | | |
| 2 | 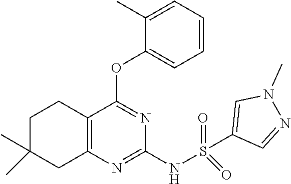 [SMILES: Cc1ccccc1Oc1nc(NS(=O)(=O)c2cnn(C)c2)nc2c1CCC(C)(C)C2] | ++ | +++ | | |
| 3 | 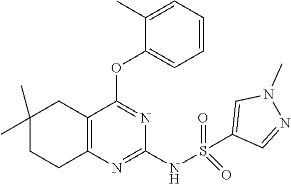 [SMILES: Cc1ccccc1Oc1nc(NS(=O)(=O)c2cnn(C)c2)nc2c1CC(C)(C)CC2] | ++ | +++ | | |
| 4 | 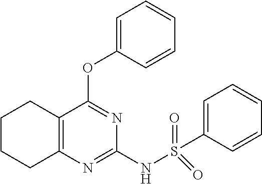 [SMILES: O=S(=O)(Nc1nc2c(c(Oc3ccccc3)n1)CCCC2)c1ccccc1] | + | +++ | | |
| 5 | 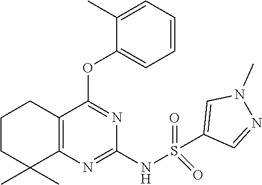 [SMILES: Cc1ccccc1Oc1nc(NS(=O)(=O)c2cnn(C)c2)nc2c1CCCC2(C)C] | + | ++ | | |
| 6 | 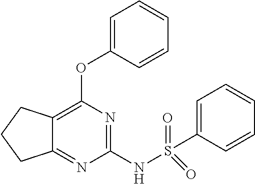 [SMILES: O=S(=O)(Nc1nc2c(c(Oc3ccccc3)n1)CCC2)c1ccccc1] | + | +++ | | |
| 7 | 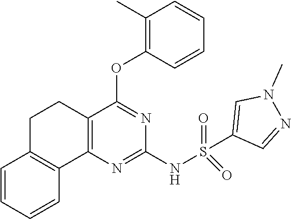 [SMILES: Cc1ccccc1Oc1nc(NS(=O)(=O)c2cnn(C)c2)nc2c1CCc1ccccc1-2] | +++ | +++ | | |
| 8 | 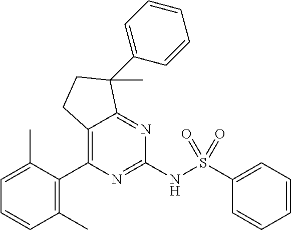 [SMILES: Cc1cccc(C)c1-c1nc(NS(=O)(=O)c2ccccc2)nc2c1CCC2(C)c1ccccc1] | | | | |
| 9 | 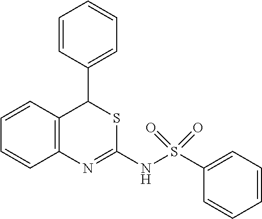 [SMILES: O=S(=O)(NC1=Nc2ccccc2C(c2ccccc2)S1)c1ccccc1] | + | ++ | | |
| 10 | 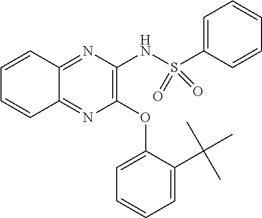 [SMILES: CC(C)(C)c1ccccc1Oc1nc2ccccc2nc1NS(=O)(=O)c1ccccc1] | +++ | ++ | | |
| 11 | 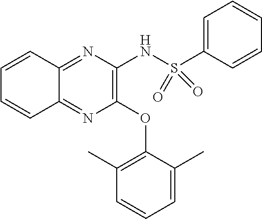 [SMILES: Cc1cccc(C)c1Oc1nc2ccccc2nc1NS(=O)(=O)c1ccccc1] | + | ++ | | |
| 12 | 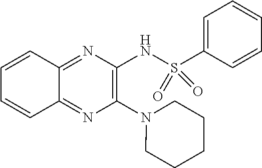 [SMILES: O=S(=O)(Nc1nc2ccccc2nc1N1CCCCC1)c1ccccc1] | + | ++ | | |
| 13 | 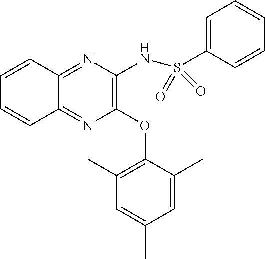 [SMILES: Cc1cc(C)c(Oc2nc3ccccc3nc2NS(=O)(=O)c2ccccc2)c(C)c1] | ++ | ++ | | |
| 14 | 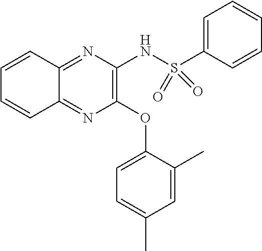 [SMILES: Cc1ccc(Oc2nc3ccccc3nc2NS(=O)(=O)c2ccccc2)c(C)c1] | ++ | ++ | | |
| 15 | 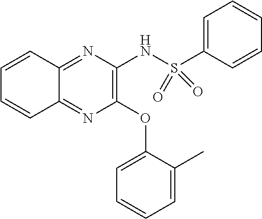 [SMILES: Cc1ccccc1Oc1nc2ccccc2nc1NS(=O)(=O)c1ccccc1] | + | ++ | | |
| 16 | 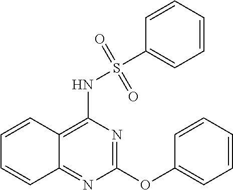 [SMILES: O=S(=O)(Nc1nc(Oc2ccccc2)nc2ccccc12)c1ccccc1] | ND | + | | |
| 17 | 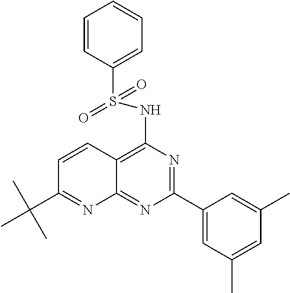 [SMILES: Cc1cc(C)cc(-c2nc(NS(=O)(=O)c3ccccc3)c3ccc(C(C)(C)C)nc3n2)c1] | ND | + | | |
| 18 | 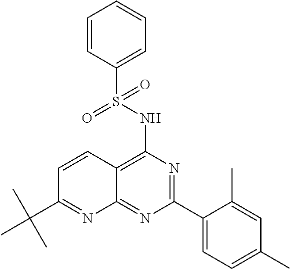 [SMILES: Cc1ccc(-c2nc(NS(=O)(=O)c3ccccc3)c3ccc(C(C)(C)C)nc3n2)c(C)c1] | ND | + | | |
| 19 | 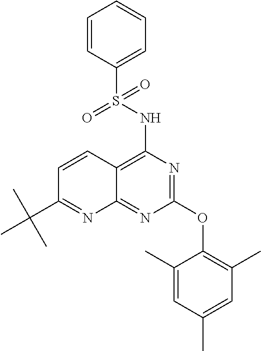 [SMILES: Cc1cc(C)c(Oc2nc(NS(=O)(=O)c3ccccc3)c3ccc(C(C)(C)C)nc3n2)c(C)c1] | ND | + | | |
| 20 | 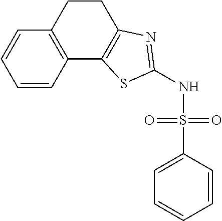 [SMILES: O=S(=O)(Nc1nc2c(s1)-c1ccccc1CC2)c1ccccc1] | + | +++ | | |
| 21 | 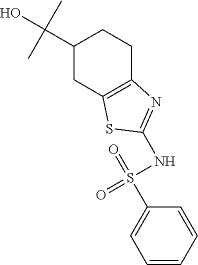 [SMILES: CC(C)(O)C1CCc2nc(NS(=O)(=O)c3ccccc3)sc2C1] | ND | + | | |
| 22 | 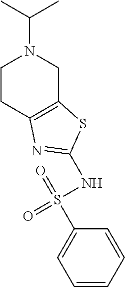 [SMILES: CC(C)N1CCc2nc(NS(=O)(=O)c3ccccc3)sc2C1] | ND | + | | |
| 23 | 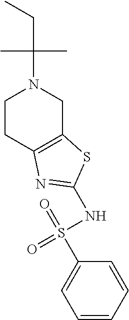 [SMILES: CCC(C)(C)N1CCc2nc(NS(=O)(=O)c3ccccc3)sc2C1] | ++ | ++ | ND | ND |
| 24 | 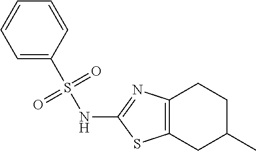 [SMILES: CC1CCc2nc(NS(=O)(=O)c3ccccc3)sc2C1] | ND | + | | |
| 25 | 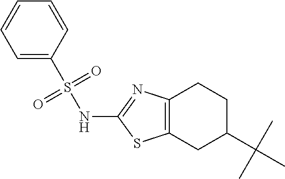 [SMILES: CC(C)(C)C1CCc2nc(NS(=O)(=O)c3ccccc3)sc2C1] | ++ | ++ | | |
| 26 | 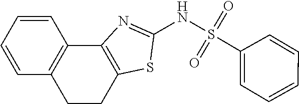 [SMILES: O=S(=O)(Nc1nc2c(s1)CCc1ccccc1-2)c1ccccc1] | ++ | ++ | | |
| 27 | 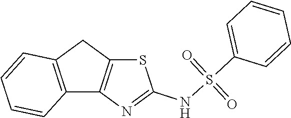 [SMILES: O=S(=O)(Nc1nc2c(s1)Cc1ccccc1-2)c1ccccc1] | + | ++ | | |
| 28 | 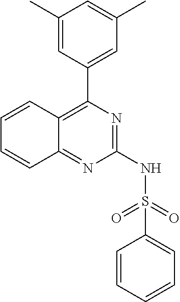 [SMILES: Cc1cc(C)cc(-c2nc(NS(=O)(=O)c3ccccc3)nc3ccccc23)c1] | ++ | +++ | | |
| 29 | 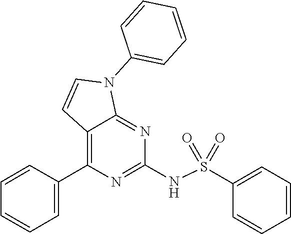 [SMILES: O=S(=O)(Nc1nc(-c2ccccc2)c2ccn(-c3ccccc3)c2n1)c1ccccc1] | +++ | +++ | | |
| 30 | 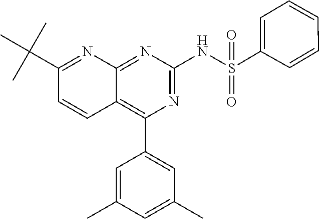 [SMILES: Cc1cc(C)cc(-c2nc(NS(=O)(=O)c3ccccc3)nc3nc(C(C)(C)C)ccc23)c1] | +++ | +++ | | |
| 31 | 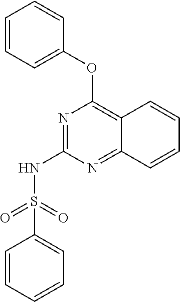 [SMILES: O=S(=O)(Nc1nc(Oc2ccccc2)c2ccccc2n1)c1ccccc1] | + | +++ | | |
| 32 | 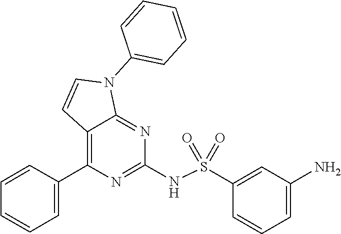 [SMILES: Nc1cccc(S(=O)(=O)Nc2nc(-c3ccccc3)c3ccn(-c4ccccc4)c3n2)c1] | +++ | +++ | | |
| 33 | 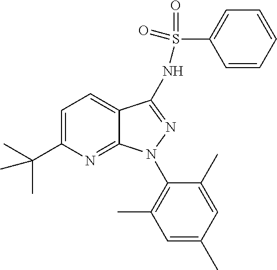 [SMILES: Cc1cc(C)c(-n2nc(NS(=O)(=O)c3ccccc3)c3ccc(C(C)(C)C)nc32)c(C)c1] | +++ | ++ | ND | + |
| 34 | 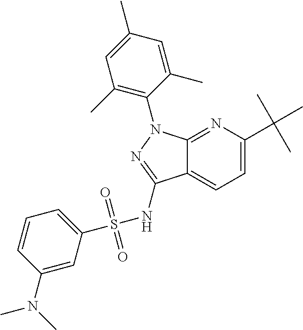 [SMILES: Cc1cc(C)c(-n2nc(NS(=O)(=O)c3cccc(N(C)C)c3)c3ccc(C(C)(C)C)nc32)c(C)c1] | +++ | +++ | | |
| 35 | 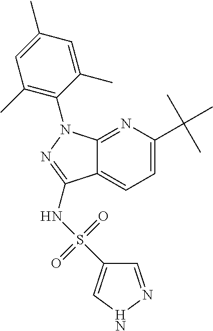 [SMILES: Cc1cc(C)c(-n2nc(NS(=O)(=O)c3cn[nH]c3)c3ccc(C(C)(C)C)nc32)c(C)c1] | ++ | +++ | | |
| 36 | 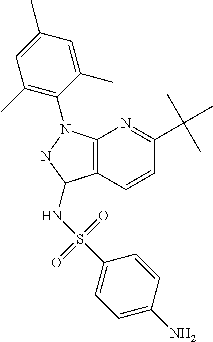 [SMILES: Cc1cc(C)c(N2NC(NS(=O)(=O)c3ccc(N)cc3)c3ccc(C(C)(C)C)nc32)c(C)c1] | ++ | ++ | | |
| 37 | 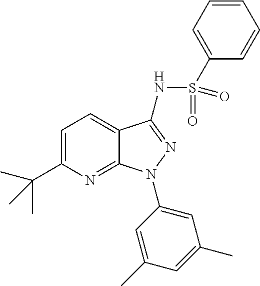 [SMILES: Cc1cc(C)cc(-n2nc(NS(=O)(=O)c3ccccc3)c3ccc(C(C)(C)C)nc32)c1] | +++ | ++ | | |
| 38 | 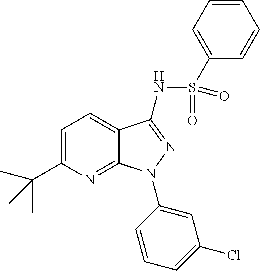 [SMILES: CC(C)(C)c1ccc2c(NS(=O)(=O)c3ccccc3)nn(-c3cccc(Cl)c3)c2n1] | +++ | ++ | | |
| 39 | 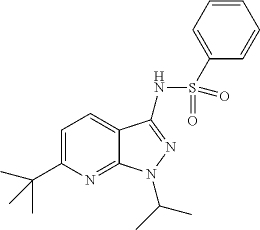 [SMILES: CC(C)n1nc(NS(=O)(=O)c2ccccc2)c2ccc(C(C)(C)C)nc21] | ++ | ++ | | |
| 40 | 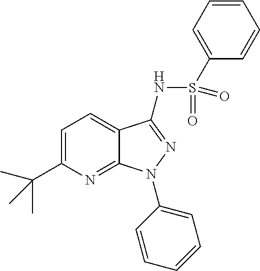 [SMILES: CC(C)(C)c1ccc2c(NS(=O)(=O)c3ccccc3)nn(-c3ccccc3)c2n1] | +++ | ++ | | |
| 41 | 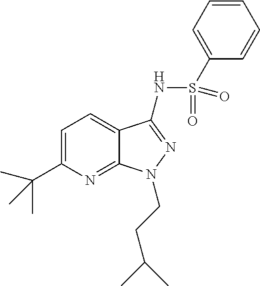 [SMILES: CC(C)CCn1nc(NS(=O)(=O)c2ccccc2)c2ccc(C(C)(C)C)nc21] | ND | + | | |
| 42 | 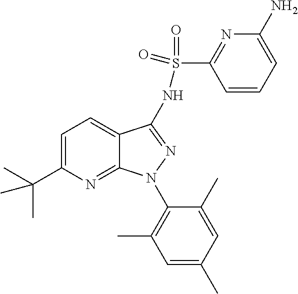 [SMILES: Cc1cc(C)c(-n2nc(NS(=O)(=O)c3cccc(N)n3)c3ccc(C(C)(C)C)nc32)c(C)c1] | ++ | ++ | | |
| 43 | 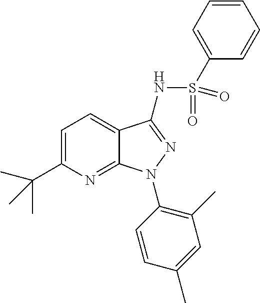 [SMILES: Cc1ccc(-n2nc(NS(=O)(=O)c3ccccc3)c3ccc(C(C)(C)C)nc32)c(C)c1] | +++ | ++ | | |
| 44 | 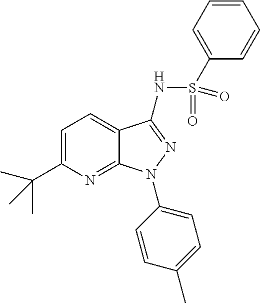 [SMILES: Cc1ccc(-n2nc(NS(=O)(=O)c3ccccc3)c3ccc(C(C)(C)C)nc32)cc1] | +++ | ++ | | |
| 45 | 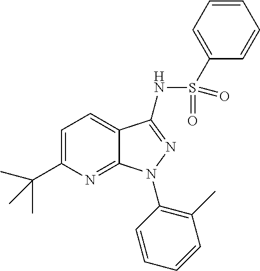 [SMILES: Cc1ccccc1-n1nc(NS(=O)(=O)c2ccccc2)c2ccc(C(C)(C)C)nc21] | +++ | ++ | | |
| 46 | 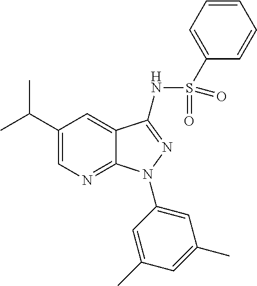 [SMILES: Cc1cc(C)cc(-n2nc(NS(=O)(=O)c3ccccc3)c3cc(C(C)C)cnc32)c1] | +++ | ++ | | |
| 47 | 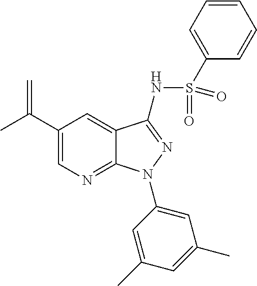 [SMILES: C=C(C)c1cnc2c(c1)c(NS(=O)(=O)c1ccccc1)nn2-c1cc(C)cc(C)c1] | +++ | ++ | | |
| 48 | 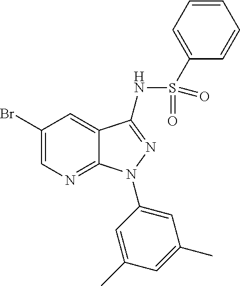 [SMILES: Cc1cc(C)cc(-n2nc(NS(=O)(=O)c3ccccc3)c3cc(Br)cnc32)c1] | +++ | ++ | | |
| 49 | 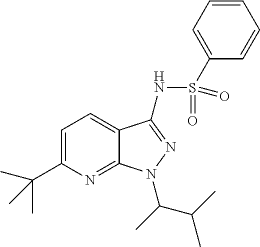 [SMILES: CC(C)C(C)n1nc(NS(=O)(=O)c2ccccc2)c2ccc(C(C)(C)C)nc21] | ND | + | | |
| 50 | 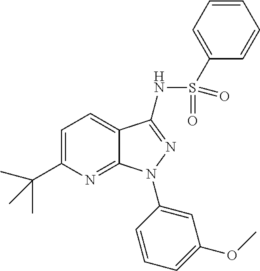 [SMILES: COc1cccc(-n2nc(NS(=O)(=O)c3ccccc3)c3ccc(C(C)(C)C)nc32)c1] | +++ | ++ | | |
| 51 | 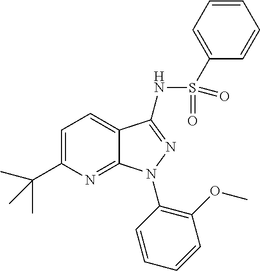 [SMILES: COc1ccccc1-n1nc(NS(=O)(=O)c2ccccc2)c2ccc(C(C)(C)C)nc21] | ++ | ++ | | |
| 52 | 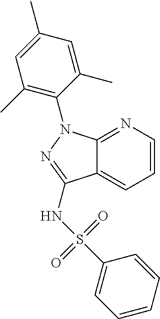 [SMILES: Cc1cc(C)c(-n2nc(NS(=O)(=O)c3ccccc3)c3cccnc32)c(C)c1] | ND | ++ | | |
| 53 | 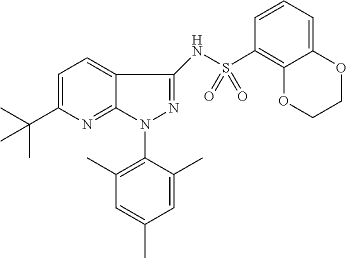 [SMILES: Cc1cc(C)c(-n2nc(NS(=O)(=O)c3cccc4c3OCCO4)c3ccc(C(C)(C)C)nc32)c(C)c1] | +++ | ++ | | |
| 54 | 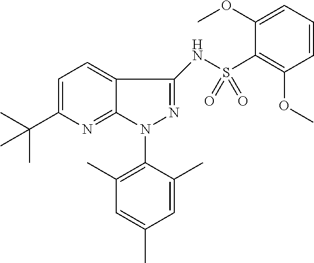 [SMILES: COc1cccc(OC)c1S(=O)(=O)Nc1nn(-c2c(C)cc(C)cc2C)c2nc(C(C)(C)C)ccc12] | ND | + | | |
| 55 | 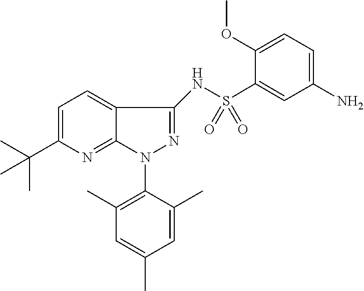 [SMILES: COc1ccc(N)cc1S(=O)(=O)Nc1nn(-c2c(C)cc(C)cc2C)c2nc(C(C)(C)C)ccc12] | + | ++ | | |
| 56 | 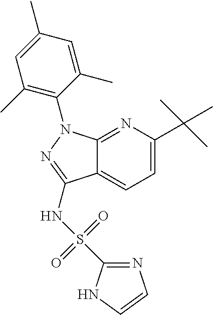 [SMILES: Cc1cc(C)c(-n2nc(NS(=O)(=O)c3ncc[nH]3)c3ccc(C(C)(C)C)nc32)c(C)c1] | ++ | ++ | | |
| 57 | 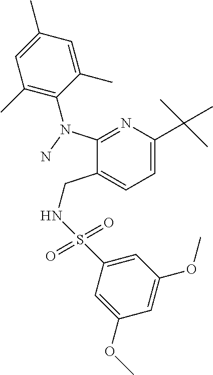 [SMILES: COc1cc(OC)cc(S(=O)(=O)NCc2ccc(C(C)(C)C)nc2N(N)c2c(C)cc(C)cc2C)c1] | ND | + | | |
| 58 | 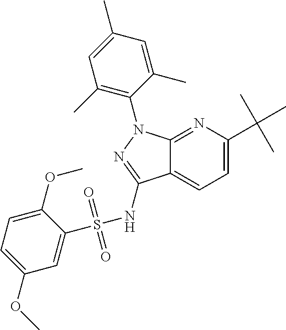 [SMILES: COc1ccc(OC)c(S(=O)(=O)Nc2nn(-c3c(C)cc(C)cc3C)c3nc(C(C)(C)C)ccc23)c1] | +++ | ++ | | |
| 59 | 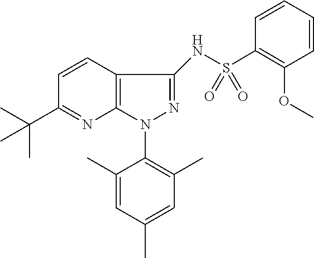 [SMILES: COc1ccccc1S(=O)(=O)Nc1nn(-c2c(C)cc(C)cc2C)c2nc(C(C)(C)C)ccc12] | ++ | ++ | | |
| 60 | 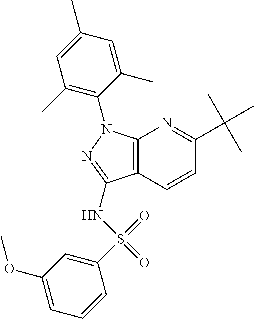 [SMILES: COc1cccc(S(=O)(=O)Nc2nn(-c3c(C)cc(C)cc3C)c3nc(C(C)(C)C)ccc23)c1] | +++ | ++ | | |
| 61 | 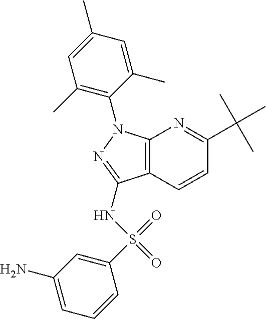 [SMILES: Cc1cc(C)c(-n2nc(NS(=O)(=O)c3cccc(N)c3)c3ccc(C(C)(C)C)nc32)c(C)c1] | +++ | ++ | | |
| 62 | 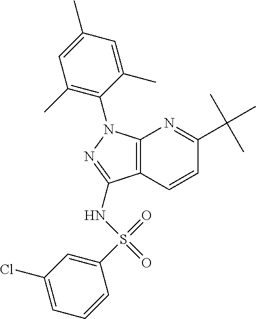 [SMILES: Cc1cc(C)c(-n2nc(NS(=O)(=O)c3cccc(Cl)c3)c3ccc(C(C)(C)C)nc32)c(C)c1] | ND | + | | |
| 63 | 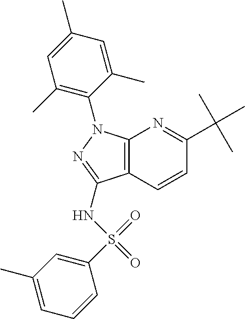 [SMILES: Cc1cccc(S(=O)(=O)Nc2nn(-c3c(C)cc(C)cc3C)c3nc(C(C)(C)C)ccc23)c1] | ND | + | | |
| 64 | 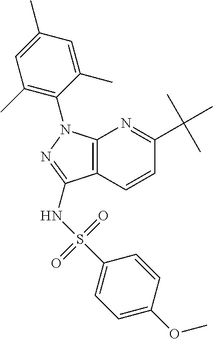 [SMILES: COc1ccc(S(=O)(=O)Nc2nn(-c3c(C)cc(C)cc3C)c3nc(C(C)(C)C)ccc23)cc1] | ND | + | | |
| 65 | 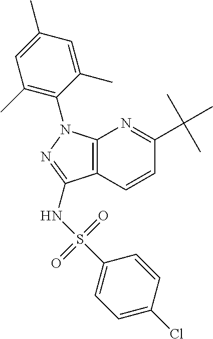 [SMILES: Cc1cc(C)c(-n2nc(NS(=O)(=O)c3ccc(Cl)cc3)c3ccc(C(C)(C)C)nc32)c(C)c1] | ND | + | | |
| 66 | 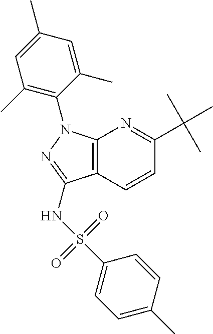 [SMILES: Cc1ccc(S(=O)(=O)Nc2nn(-c3c(C)cc(C)cc3C)c3nc(C(C)(C)C)ccc23)cc1] | ND | + | | |
| 67 | 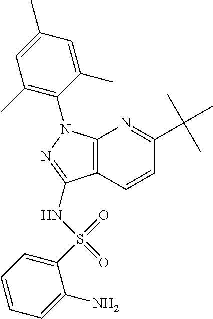 [SMILES: Cc1cc(C)c(-n2nc(NS(=O)(=O)c3ccccc3N)c3ccc(C(C)(C)C)nc32)c(C)c1] | ND | + | | |
| 68 | 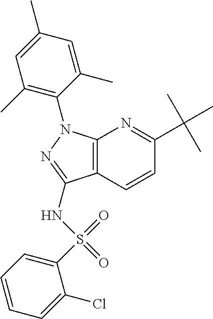 [SMILES: Cc1cc(C)c(-n2nc(NS(=O)(=O)c3ccccc3Cl)c3ccc(C(C)(C)C)nc32)c(C)c1] | ND | + | | |
| 69 | 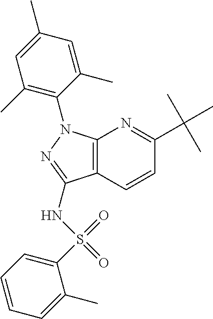 [SMILES: Cc1cc(C)c(-n2nc(NS(=O)(=O)c3ccccc3C)c3ccc(C(C)(C)C)nc32)c(C)c1] | ND | + | | |
| 70 | 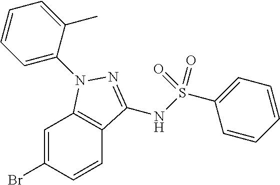 [SMILES: Cc1ccccc1-n1nc(NS(=O)(=O)c2ccccc2)c2ccc(Br)cc21] | ++ | ++ | | |
| 71 | 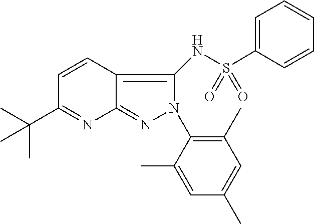 [SMILES: Cc1cc(C)c(-n2nc3nc(C(C)(C)C)ccc3c2NS(=O)(=O)c2ccccc2)c(C)c1] | ND | + | | |
| 72 | 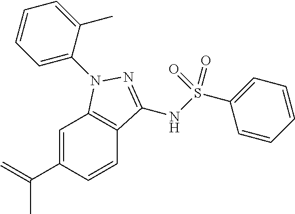 [SMILES: C=C(C)c1ccc2c(NS(=O)(=O)c3ccccc3)nn(-c3ccccc3C)c2c1] | ++ | +++ | | |
| 73 | 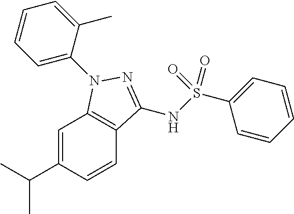 [SMILES: Cc1ccccc1-n1nc(NS(=O)(=O)c2ccccc2)c2ccc(C(C)C)cc21] | ++ | ++ | | |
| 74 | 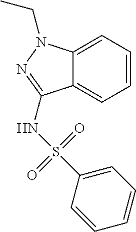 [SMILES: CCn1nc(NS(=O)(=O)c2ccccc2)c2ccccc21] | ND | + | | |
| 75 | 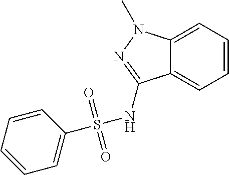 [SMILES: Cn1nc(NS(=O)(=O)c2ccccc2)c2ccccc21] | ND | ND | | |
| 76 | 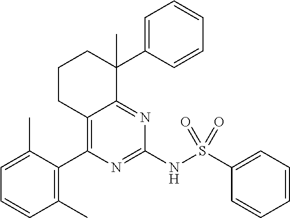 [SMILES: Cc1cccc(C)c1-c1nc(NS(=O)(=O)c2ccccc2)nc2c1CCCC2(C)c1ccccc1] | +++ | +++ | | |
| 77 | 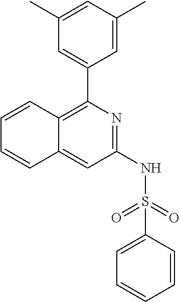 [SMILES: Cc1cc(C)cc(-c2nc(NS(=O)(=O)c3ccccc3)cc3ccccc23)c1] | +++ | +++ | | |
| 78 | 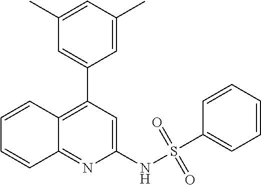 [SMILES: Cc1cc(C)cc(-c2cc(NS(=O)(=O)c3ccccc3)nc3ccccc23)c1] | ND | + | | |
| 79 | 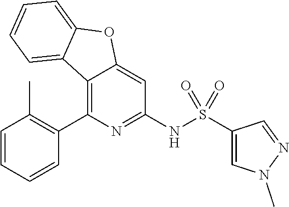 [SMILES: Cc1ccccc1-c1nc(NS(=O)(=O)c2cnn(C)c2)cc2oc3ccccc3c12] | | | + | ++ |
| 80 | 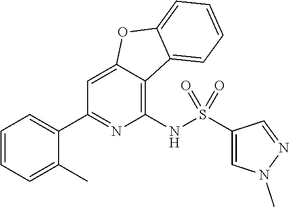 [SMILES: Cc1ccccc1-c1cc2oc3ccccc3c2c(NS(=O)(=O)c2cnn(C)c2)n1] | | | ND | + |
| 81 | 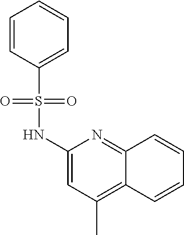 [SMILES: Cc1cc(NS(=O)(=O)c2ccccc2)nc2ccccc12] | ND | + | | |
| 82 | 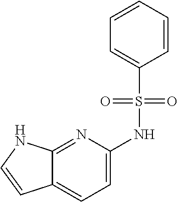 [SMILES: O=S(=O)(Nc1ccc2cc[nH]c2n1)c1ccccc1] | ND | + | | |
| 83 | 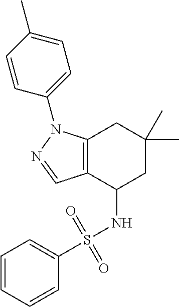 [SMILES: Cc1ccc(-n2ncc3c2CC(C)(C)CC3NS(=O)(=O)c2ccccc2)cc1] | ND | + | | |
| 84 | 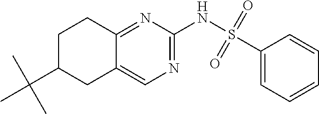 [SMILES: CC(C)(C)C1CCc2nc(NS(=O)(=O)c3ccccc3)ncc2C1] | ND | + | | |
| 85 | 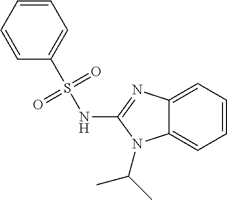 [SMILES: CC(C)n1c(NS(=O)(=O)c2ccccc2)nc2ccccc21] | ND | + | | |
| 86 | 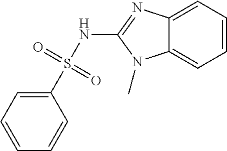 [SMILES: Cn1c(NS(=O)(=O)c2ccccc2)nc2ccccc21] | ND | + | | |
| 87 | 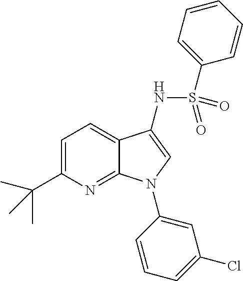 [SMILES: CC(C)(C)c1ccc2c(NS(=O)(=O)c3ccccc3)cn(-c3cccc(Cl)c3)c2n1] | ND | + | | |
| 88 | 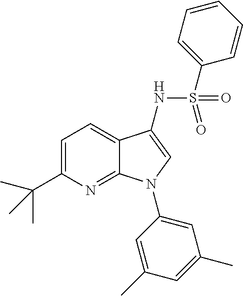 [SMILES: Cc1cc(C)cc(-n2cc(NS(=O)(=O)c3ccccc3)c3ccc(C(C)(C)C)nc32)c1] | ND | + | | |
| 89 | 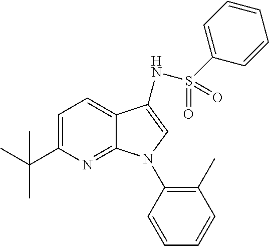 [SMILES: Cc1ccccc1-n1cc(NS(=O)(=O)c2ccccc2)c2ccc(C(C)(C)C)nc21] | ND | + | | |
| 90 | 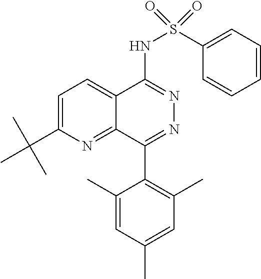 [SMILES: Cc1cc(C)c(-c2nnc(NS(=O)(=O)c3ccccc3)c3ccc(C(C)(C)C)nc23)c(C)c1] | ND | + | | |
| 91 | 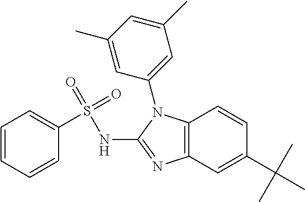 [SMILES: Cc1cc(C)cc(-n2c(NS(=O)(=O)c3ccccc3)nc3cc(C(C)(C)C)ccc32)c1] | ND | + | | |
| 92 | 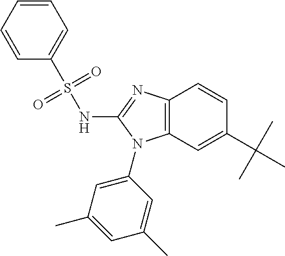 [SMILES: Cc1cc(C)cc(-n2c(NS(=O)(=O)c3ccccc3)nc3ccc(C(C)(C)C)cc32)c1] | ND | + | | |
| 93 | 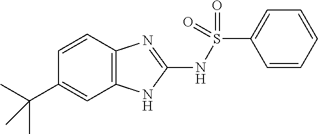 [SMILES: CC(C)(C)c1ccc2nc(NS(=O)(=O)c3ccccc3)[nH]c2c1] | ND | + | | |
| 94 | 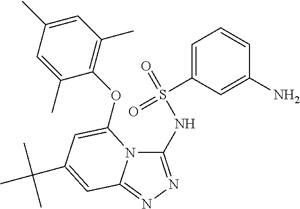 [SMILES: Cc1cc(C)c(Oc2cc(C(C)(C)C)cc3nnc(NS(=O)(=O)c4cccc(N)c4)n23)c(C)c1] | ND | + | | |
| 95 | 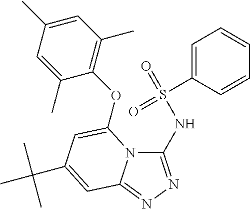 [SMILES: Cc1cc(C)c(Oc2cc(C(C)(C)C)cc3nnc(NS(=O)(=O)c4ccccc4)n23)c(C)c1] | ND | + | | |

### VI. Synthesis of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before fourrier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO2 rotors and spun under Magic Angle Spinning (MAS) condition with spinning speed typically set to 12.5 kHz. The proton relaxation time was measured using 1H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)+5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hour then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. ¹⁹F NMR (282 MHz, CDCl₃) δ−63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 μM. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ −63.9 (s, 3F) ppm. ESI-MS m/z calc. 498.06134, Retention time: 2.34 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2.000 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et₂O (270 mL) at −78° C. was added bromo(but-3-enyl)magnesium in THE (190 mL of 0.817 M, 155.23 mmol) dropwise over a period of 1.5 h (inner temperature −72° C. to −76° C.). The mixture was stirred at −78° C. for 20 min. The dry ice-acetone bath was removed. The mixture was slowly warm to 5° C. during 1 h, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃(50 mL) and brine (20 mL), dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ −78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 min. Benzyl bromide (24.13 g, 141.08 mmol) and TBAI (8.68 g, 23.500 mmol) were added. The mixture was stirred at room temperature overnight. NH₄Cl (3 g, 0.6 eq) was added. The mixture was stirred for 10 min. 30 mL of EtOAc was added, then ice-water was added (400 g). The mixture was extracted with CH₂Cl₂ and the combined organic layers were concentrated. Purification by silica gel chromatography (0-20% CH₂Cl₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. ¹⁹F NMR (282 MHz, CDCl₃) δ−70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ−70.29 (s, 3F) ppm. ESI-MS m/z calc. 288.09732, found 287.1 (M-1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ −71.63 (br s, 3F) ppm. ESI-MS m/z calc. 288.0973, found 287.0 (M-1); Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over MgSO₄, filtered and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 μm particle) made by Waters (pn: 186002350) and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with IL of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T₃P (622 g of 50% w/w, 977.4 mmol) using an ice-water bath to keep the temperature <35° C. (temperature rose to 34° C.) and the reaction mixture was stirred at ambient temperature for 18 h. Added additional DIEA (100 mL, 574.1 mmol) and T₃P (95 g, 298.6 mmol) and stirred at ambient temperature for 2 days. Starting material was still observed and an additional T₃P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotovap bath until dissolved, then evaporated out 325 mL of solvent by rotary evaporation. The rotovap bath temp was allowed to drop to room temperature and product started crystallizing out during the evaporation. Then put the flask in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed water (3×3 L) and with brine (1×2.5 L). The organic phase was dried with MgSO₄, filtered and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotovap at 100 mbar pressure at room temperature for 1.5 h. The solid mass was stirred mechanically for 2 h at room temperature, resultant thick fine suspension was filtered, washed with dry ice cold heptane and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a course porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached ˜75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-Amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5]nonadeca-1(18),2,4,14,16-pentaen-17-yl]carbamate an off-white solid. 1H NMR and MS confirmed the expected product. CFTR modulating activity was confirmed using a standard Ussing Chamber Assay for CFTR potentiator activity.

### Other Embodiments

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound represented by the following structural formula: 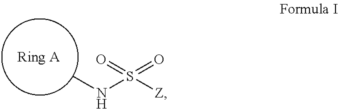
a tautomer thereof, a deuterated derivative of that compound or tautomer, or a pharmaceutically acceptable salt of any of the foregoing, wherein:
Ring A is a bicyclic or tricyclic ring system selected from: 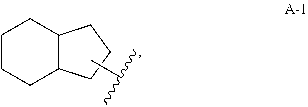
wherein Ring A-1 contains one or more unsaturated bonds and 2 to 3 ring carbon atoms that are independently replaced by nitrogen or sulfur; 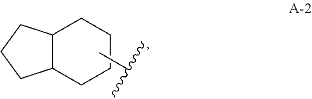
wherein Ring A-2 contains one or more unsaturated bonds and 1 to 3 ring carbon atoms that are replaced by nitrogen; 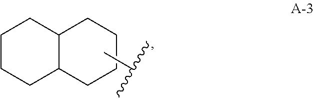
wherein Ring A-3 contains one or more unsaturated bonds and 1 to 3 ring carbon atoms that are independently replaced by nitrogen or sulfur; 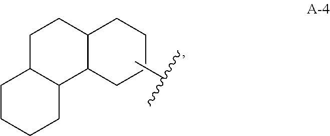
wherein Ring A-4 contains one or more unsaturated bonds and 1 to 3 ring carbon atoms that are replaced by nitrogen; 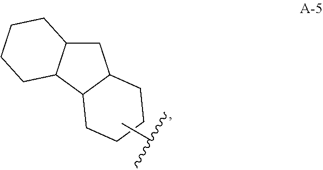
wherein Ring A-5 contains one or more unsaturated bonds and 1 to 3 ring carbon atoms that are independently replaced by nitrogen or oxygen; 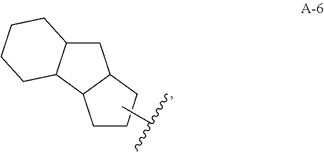
wherein Ring A-6 contains one or more unsaturated bonds and 2 to 3 ring carbon atoms that are independently replaced by nitrogen or sulfur; and 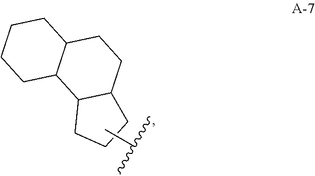
wherein Ring A-7 contains one or more unsaturated bonds and 2 to 3 ring carbon atoms that are independently replaced by nitrogen or sulfur;
Ring A is optionally substituted with 1 to 3 R¹ groups; wherein each R¹ is independently selected from:
halogen,
—C₁-C₆ alkyl optionally substituted with 1 to 3 groups selected from halogen, —OH and —C₁-C₄ alkoxy;
phenyl optionally substituted with 1 to 3 groups selected from halogen, —CN, —C₁-C₆ alkyl (which is optionally further substituted with 1 to 3 groups selected from halogen and —OH), and —C₁-C₆ alkoxy;
—O-phenyl optionally substituted with 1 to 3 groups selected from halogen, —CN, and —C₁-C₆ alkyl (which is optionally further substituted with 1 to 3 groups selected from halogen and —OH); and
piperidinyl; and
Z is selected from:
phenyl optionally substituted with 1 or 2 groups independently selected from —NH₂ (optionally substituted with 1-2 groups selected from —C₁-C₃ alkyl), —C₁-C₃ alkyl, and —C₁-C₃ alkoxy;
pyrazolyl optionally substituted with 1 or 2 groups independently selected from halogen, —C₁-C₃ alkyl, and —C₁-C₃ alkoxy;
imidazolyl;
1,4 benzodioxanyl; and
pyridinyl optionally substituted with NH₂;
provided that the compound is not one of the following:
4-methyl-N-(1-methylisoquinolin-3-yl)benzenesulfonamide;
N-(4,5,6,7-tetrahydro-6-methyl-2-benzothiazolyl)benzenesulfonamide; and
N-[4,5,6,7-tetrahydro-1-(2-pyridinyl)-1H-indazol-4-yl]benzenesulfonamide.

2. The compound, tautomer, deuterated derivative, or pharmaceutically acceptable salt according to claim 1, wherein Ring A is selected from: 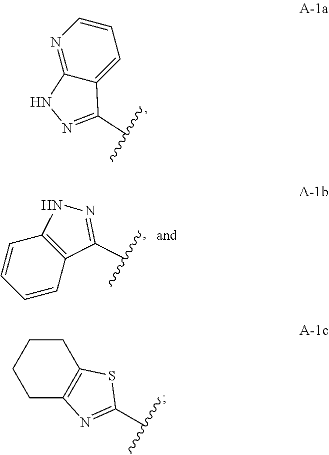
wherein each of A-1a through A-1c is optionally substituted with 1 to 3 R¹ groups as defined in claim 1,
and wherein when Ring A is A-1b or A-1c, and Z is phenyl, Z is unsubstituted.

3. The compound, tautomer, deuterated derivative, or pharmaceutically acceptable salt according to claim 2, wherein substituted Ring A-1a is selected from:

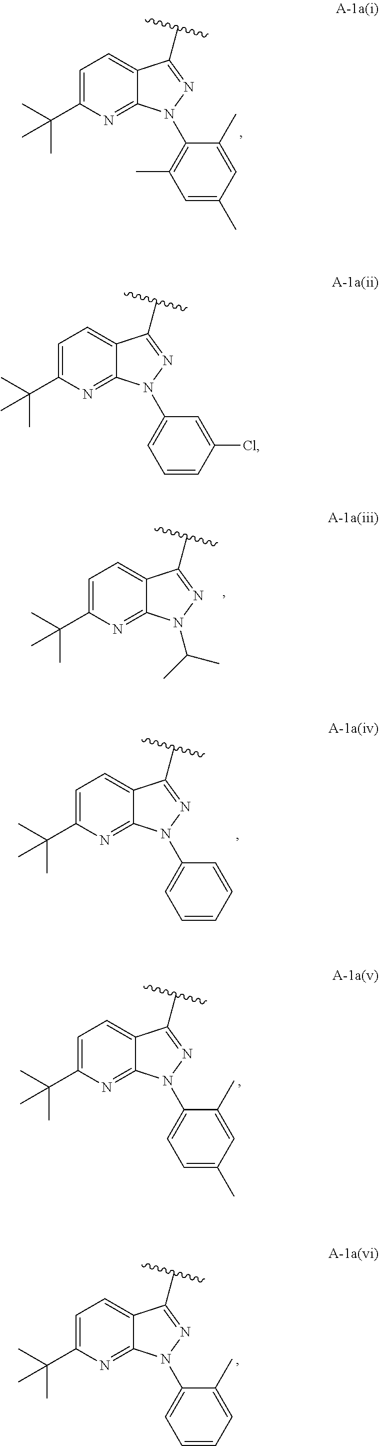

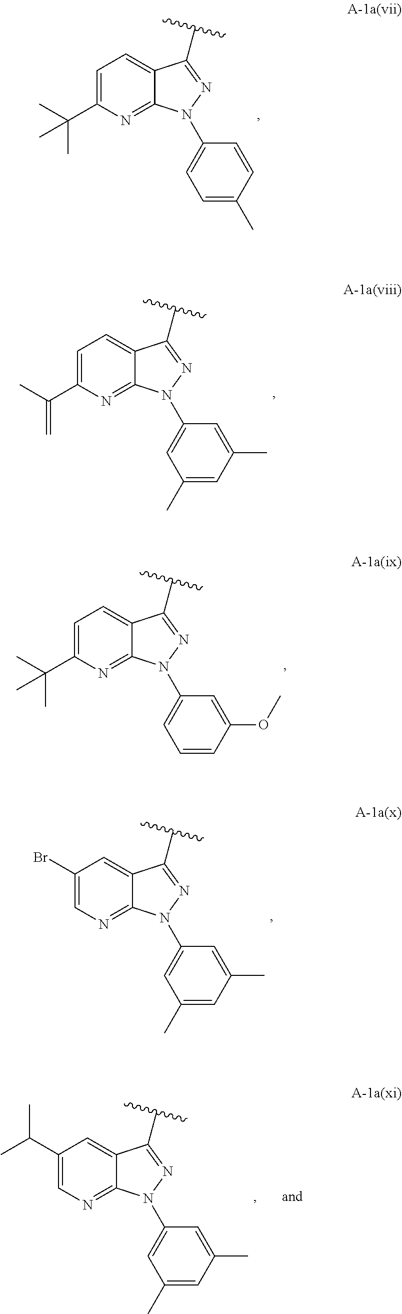

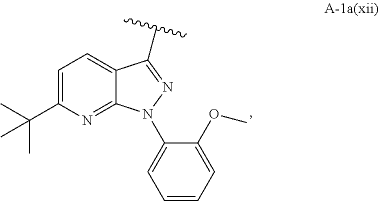 [SMILES: *c1nn(-c2ccccc2OC)c2nc(C(C)(C)C)ccc12]

4. The compound, tautomer, deuterated derivative, or pharmaceutically acceptable salt according to claim 2, wherein substituted Ring A-1b is selected from:

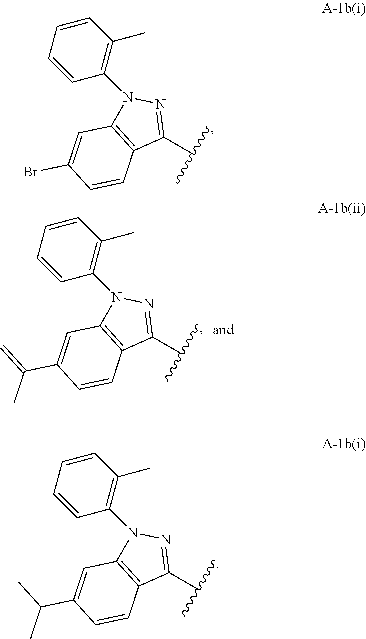

5. The compound, tautomer, deuterated derivative, or pharmaceutically acceptable salt according to claim 2, wherein substituted Ring A-1c is selected from:

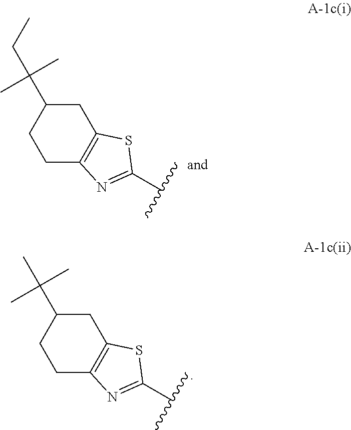 [SMILES: *c1nc2c(s1)CC(C(C)(C)CC)CC2 ; *c1nc2c(s1)CC(C(C)(C)C)CC2]

6. The compound, tautomer, deuterated derivative, or pharmaceutically acceptable salt according to claim 1, wherein Ring A is selected from: 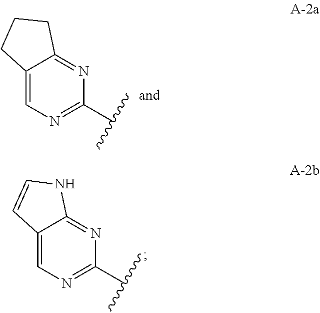 [SMILES: *c1ncc2c(n1)CCC2 ; *c1ncc2cc[nH]c2n1]
wherein each of A-2a and A-2b is optionally substituted with 1 to 3 R¹ groups as defined in claim 1,
and wherein when Ring A is A-2a and Z is phenyl, Z is unsubstituted.

7. The compound, tautomer, deuterated derivative, or pharmaceutically acceptable salt according to claim 6, wherein substituted Ring A-2a is selected from:

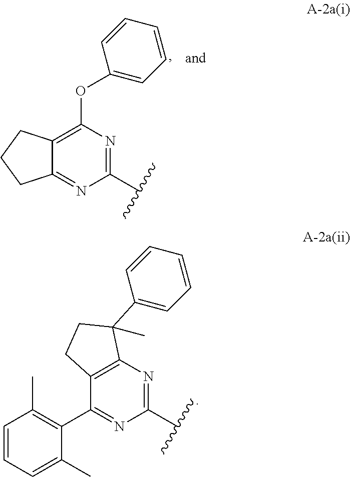 [SMILES: *c1nc2c(c(Oc3ccccc3)n1)CCC2 ; *c1nc(-c2c(C)cccc2C)c2c(n1)C(C)(c1ccccc1)CC2]

8. The compound, tautomer, deuterated derivative, or pharmaceutically acceptable salt according to claim 6, wherein substituted Ring A-2b is:

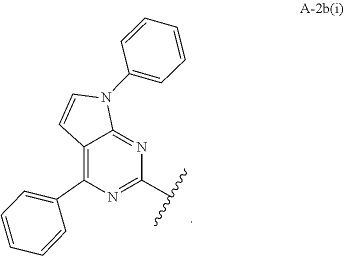 [SMILES: *c1nc(-c2ccccc2)c2ccn(-c3ccccc3)c2n1]

9. The compound, tautomer, deuterated derivative, or pharmaceutically acceptable salt according to claim 1, wherein Ring A is selected from: 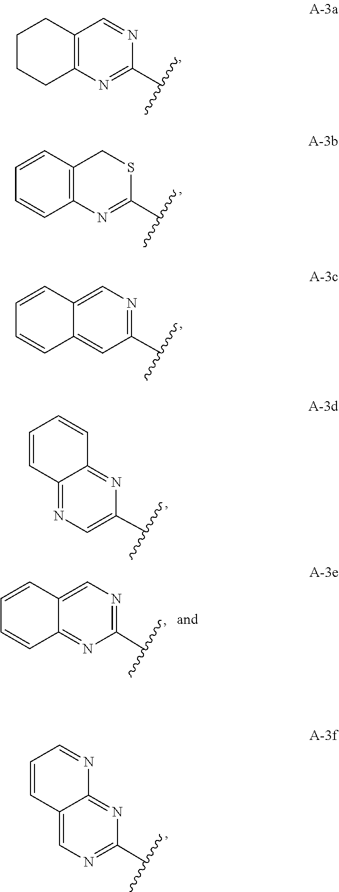 [SMILES: *c1ncc2c(n1)CCCC2 ; *C1=Nc2ccccc2CS1 ; *c1cc2ccccc2cn1 ; *c1cnc2ccccc2n1 ; *c1ncc2ccccc2n1 ; *c1ncc2cccnc2n1]
wherein each of A-3a through A-3f is optionally substituted with 1 to 3 R¹ groups as defined in claim 1,
and wherein when Ring A is A-3d or A-3e and Z is phenyl, Z is unsubstituted.

10. The compound, tautomer, deuterated derivative, or pharmaceutically acceptable salt according to claim 9, wherein substituted Ring A-3a is selected from:

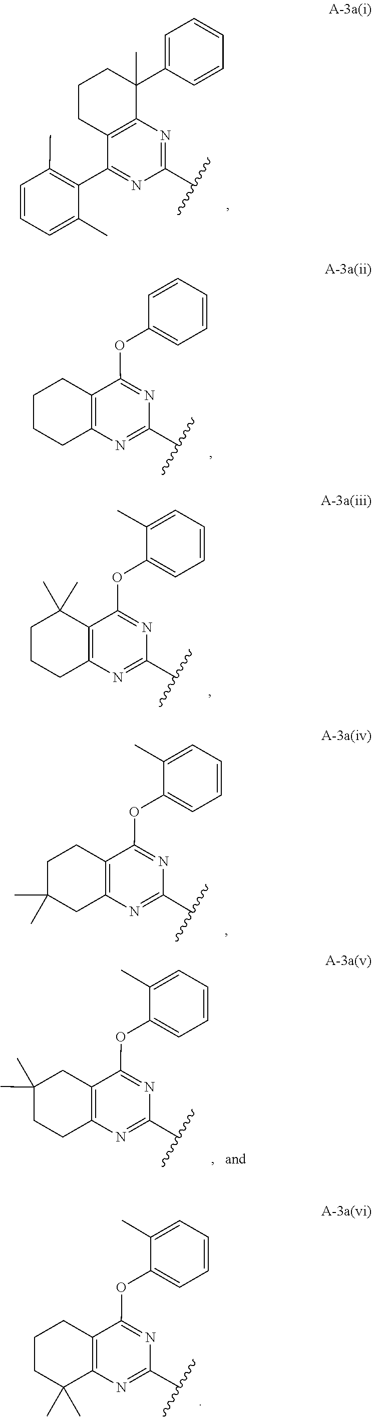 [SMILES: *c1nc(-c2c(C)cccc2C)c2c(n1)C(C)(c1ccccc1)CCC2 ; *c1nc2c(c(Oc3ccccc3)n1)CCCC2 ; *c1nc2c(c(Oc3ccccc3C)n1)C(C)(C)CCC2 ; *c1nc2c(c(Oc3ccccc3C)n1)CCC(C)(C)C2 ; *c1nc2c(c(Oc3ccccc3C)n1)CC(C)(C)CC2 ; *c1nc(Oc2ccccc2C)c2c(n1)C(C)(C)CCC2]

11. The compound, tautomer, deuterated derivative, or pharmaceutically acceptable salt according to claim 9, wherein substituted Ring A-3b is:

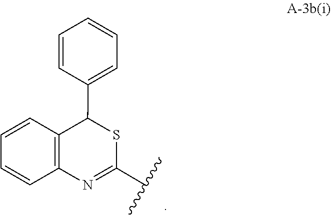 [SMILES: *C1=Nc2ccccc2C(c2ccccc2)S1]

12. The compound, tautomer, deuterated derivative, or pharmaceutically acceptable salt according to claim 9, wherein substituted Ring A-3c is:

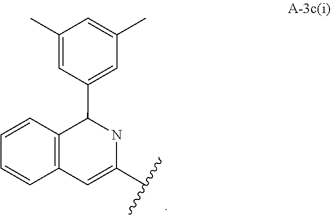 [SMILES: *C1=Cc2ccccc2C(c2cc(C)cc(C)c2)N1]

13. The compound, tautomer, deuterated derivative, or pharmaceutically acceptable salt according to claim 9, wherein substituted Ring A-3d is selected from:

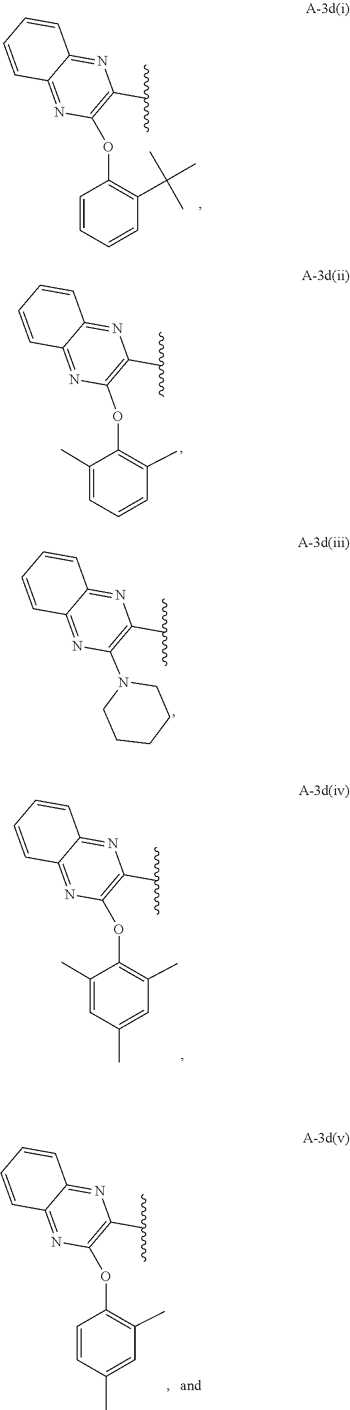 [SMILES: *c1nc2ccccc2nc1Oc1ccccc1C(C)(C)C ; *c1nc2ccccc2nc1Oc1c(C)cccc1C ; *c1nc2ccccc2nc1N1CCCCC1 ; *c1nc2ccccc2nc1Oc1c(C)cc(C)cc1C ; *c1nc2ccccc2nc1Oc1ccc(C)cc1C]

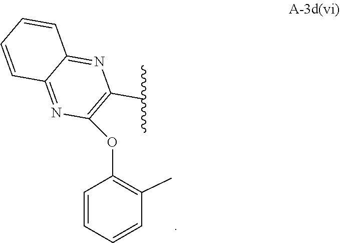 [SMILES: *c1nc2ccccc2nc1Oc1ccccc1C]

14. The compound, tautomer, deuterated derivative, or pharmaceutically acceptable salt according to claim 9, wherein substituted Ring A-3e is selected from:

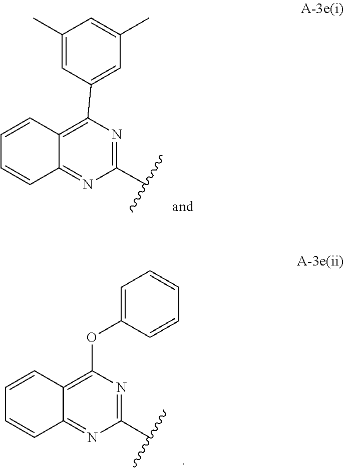 [SMILES: *c1nc(-c2cc(C)cc(C)c2)c2ccccc2n1 ; *c1nc(Oc2ccccc2)c2ccccc2n1]

15. The compound, tautomer, deuterated derivative, or pharmaceutically acceptable salt according to claim 9, wherein substituted Ring A-3f is:

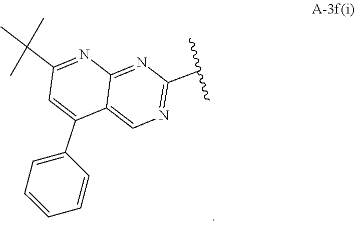 [SMILES: *c1ncc2c(-c3ccccc3)cc(C(C)(C)C)nc2n1]

16. The compound, tautomer, deuterated derivative, or pharmaceutically acceptable salt according to claim 1, wherein Ring A is: 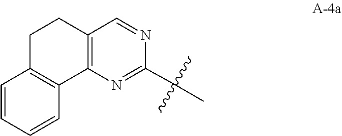 [SMILES: *c1ncc2c(n1)-c1ccccc1CC2]
optionally substituted with 1 to 3 R¹ groups as defined in claim 1.

17. The compound, tautomer, deuterated derivative, or pharmaceutically acceptable salt according to claim 15, wherein Ring A-4a is:

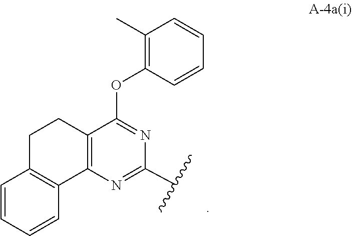 [SMILES: *c1nc(Oc2ccccc2C)c2c(n1)-c1ccccc1CC2]

18. The compound, tautomer, deuterated derivative, or pharmaceutically acceptable salt according to claim 1, wherein Ring A is selected from: 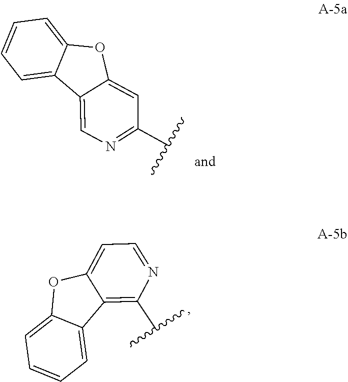 [SMILES: *c1cc2oc3ccccc3c2cn1 ; *c1nccc2oc3ccccc3c12]
wherein each of A-5a and A-5b is optionally substituted with 1 to 3 R¹ groups as defined in claim 1.

19. The compound, tautomer, deuterated derivative, or pharmaceutically acceptable salt according to claim 18, wherein Ring A-5a is:

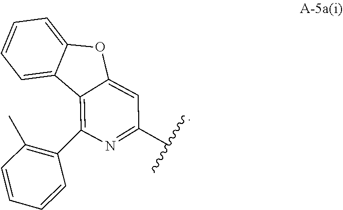 [SMILES: *c1cc2oc3ccccc3c2c(-c2ccccc2C)n1]

20. The compound, tautomer, deuterated derivative, or pharmaceutically acceptable salt according to claim 18, wherein Ring A-5b is:

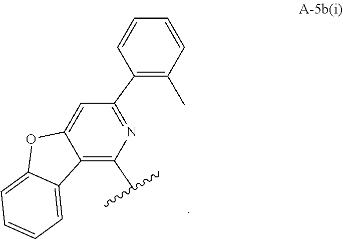 [SMILES: *c1nc(-c2ccccc2C)cc2oc3ccccc3c12]

21. The compound, tautomer, deuterated derivative, or pharmaceutically acceptable salt according to claim 1, wherein Ring A is: 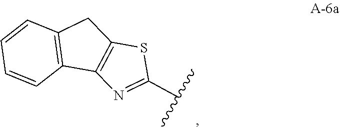 [SMILES: *c1nc2c(s1)Cc1ccccc1-2]
optionally substituted with 1 to 3 R¹ groups.

22. The compound, tautomer, deuterated derivative, or pharmaceutically acceptable salt according to claim 1, wherein Ring A is: 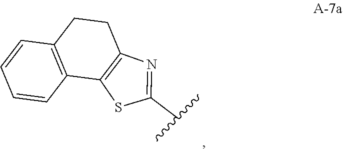 [SMILES: *c1nc2c(s1)-c1ccccc1CC2]
optionally substituted with 1 to 3 R¹ groups.

23. A compound selected from:

| Cmpd No. | Structure |
|---|---|
| 1 | 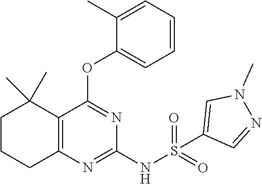 [SMILES: Cc1ccccc1Oc1nc(NS(=O)(=O)c2cnn(C)c2)nc2c1C(C)(C)CCC2] |
| 2 | 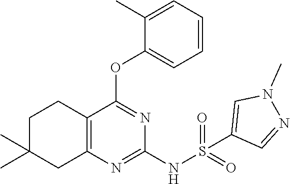 [SMILES: Cc1ccccc1Oc1nc(NS(=O)(=O)c2cnn(C)c2)nc2c1CCC(C)(C)C2] |
| 3 | 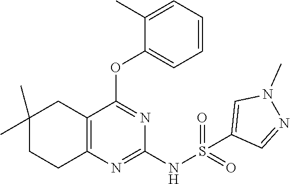 [SMILES: Cc1ccccc1Oc1nc(NS(=O)(=O)c2cnn(C)c2)nc2c1CC(C)(C)CC2] |
| 4 | 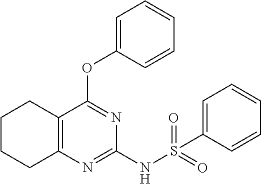 [SMILES: O=S(=O)(Nc1nc2c(c(Oc3ccccc3)n1)CCCC2)c1ccccc1] |
| 5 | 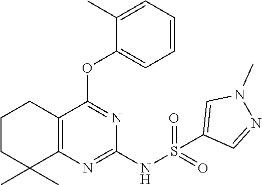 [SMILES: Cc1ccccc1Oc1nc(NS(=O)(=O)c2cnn(C)c2)nc2c1CCCC2(C)C] |
| 6 | 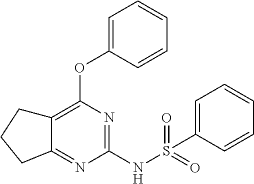 [SMILES: O=S(=O)(Nc1nc2c(c(Oc3ccccc3)n1)CCC2)c1ccccc1] |
| 7 | 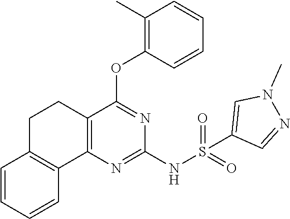 [SMILES: Cc1ccccc1Oc1nc(NS(=O)(=O)c2cnn(C)c2)nc2c1CCc1ccccc1-2] |
| 8 | 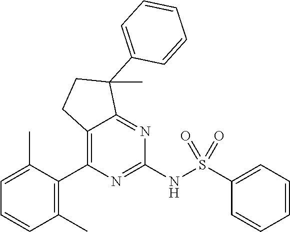 [SMILES: Cc1cccc(C)c1-c1nc(NS(=O)(=O)c2ccccc2)nc2c1CCC2(C)c1ccccc1] |
| 9 | 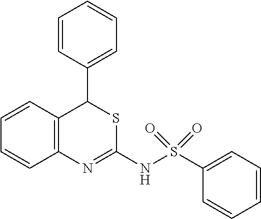 [SMILES: O=S(=O)(NC1=Nc2ccccc2C(c2ccccc2)S1)c1ccccc1] |
| 10 | 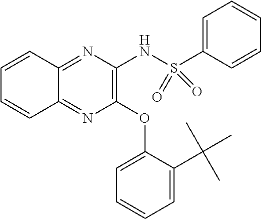 [SMILES: CC(C)(C)c1ccccc1Oc1nc2ccccc2nc1NS(=O)(=O)c1ccccc1] |
| 11 | 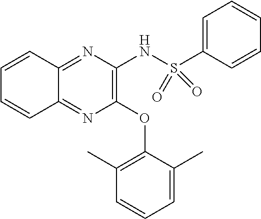 [SMILES: Cc1cccc(C)c1Oc1nc2ccccc2nc1NS(=O)(=O)c1ccccc1] |
| 12 | 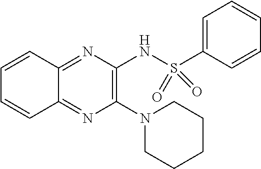 [SMILES: O=S(=O)(Nc1nc2ccccc2nc1N1CCCCC1)c1ccccc1] |
| 13 | 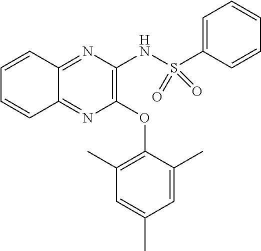 [SMILES: Cc1cc(C)c(Oc2nc3ccccc3nc2NS(=O)(=O)c2ccccc2)c(C)c1] |
| 14 | 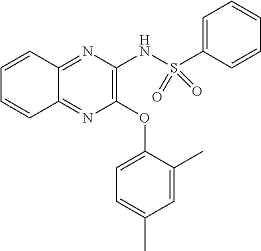 [SMILES: Cc1ccc(Oc2nc3ccccc3nc2NS(=O)(=O)c2ccccc2)c(C)c1] |
| 15 | 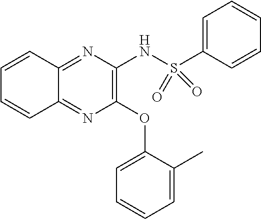 [SMILES: Cc1ccccc1Oc1nc2ccccc2nc1NS(=O)(=O)c1ccccc1] |
| 16 | 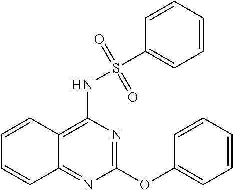 [SMILES: O=S(=O)(Nc1nc(Oc2ccccc2)nc2ccccc12)c1ccccc1] |
| 17 | 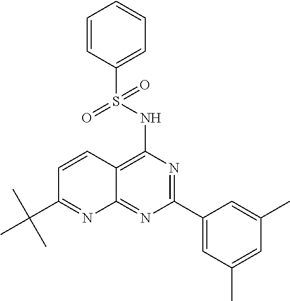 [SMILES: Cc1cc(C)cc(-c2nc(NS(=O)(=O)c3ccccc3)c3ccc(C(C)(C)C)nc3n2)c1] |
| 18 | 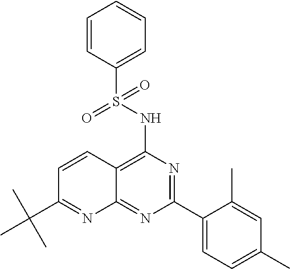 [SMILES: Cc1ccc(-c2nc(NS(=O)(=O)c3ccccc3)c3ccc(C(C)(C)C)nc3n2)c(C)c1] |
| 19 | 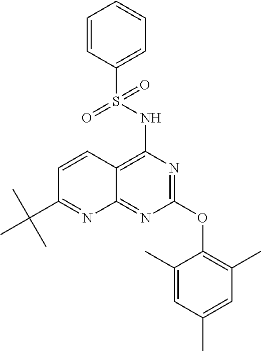 [SMILES: Cc1cc(C)c(Oc2nc(NS(=O)(=O)c3ccccc3)c3ccc(C(C)(C)C)nc3n2)c(C)c1] |
| 20 | 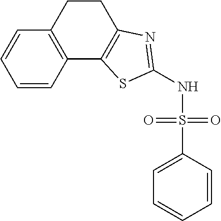 [SMILES: O=S(=O)(Nc1nc2c(s1)-c1ccccc1CC2)c1ccccc1] |
| 21 | 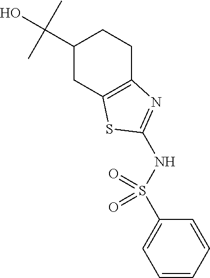 [SMILES: CC(C)(O)C1CCc2nc(NS(=O)(=O)c3ccccc3)sc2C1] |
| 22 | 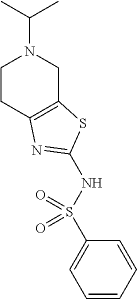 [SMILES: CC(C)N1CCc2nc(NS(=O)(=O)c3ccccc3)sc2C1] |
| 23 | 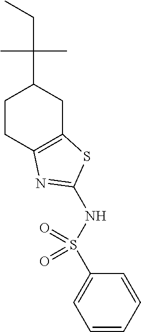 [SMILES: CCC(C)(C)C1CCc2nc(NS(=O)(=O)c3ccccc3)sc2C1] |
| 24 | 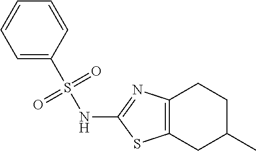 [SMILES: CC1CCc2nc(NS(=O)(=O)c3ccccc3)sc2C1] |
| 25 | 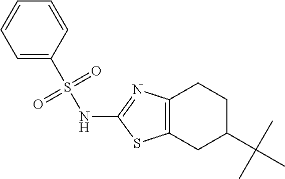 [SMILES: CC(C)(C)C1CCc2nc(NS(=O)(=O)c3ccccc3)sc2C1] |
| 26 | 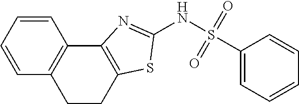 [SMILES: O=S(=O)(Nc1nc2c(s1)CCc1ccccc1-2)c1ccccc1] |
| 27 | 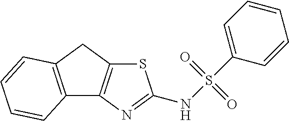 [SMILES: O=S(=O)(Nc1nc2c(s1)Cc1ccccc1-2)c1ccccc1] |
| 28 | 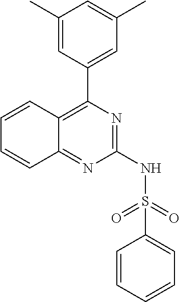 [SMILES: Cc1cc(C)cc(-c2nc(NS(=O)(=O)c3ccccc3)nc3ccccc23)c1] |
| 29 | 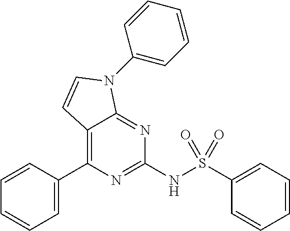 [SMILES: O=S(=O)(Nc1nc(-c2ccccc2)c2ccn(-c3ccccc3)c2n1)c1ccccc1] |
| 30 | 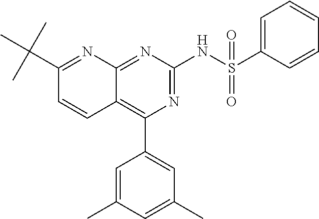 [SMILES: Cc1cc(C)cc(-c2nc(NS(=O)(=O)c3ccccc3)nc3nc(C(C)(C)C)ccc23)c1] |
| 31 | 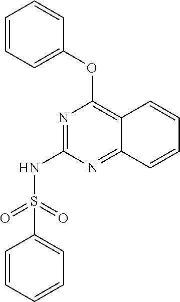 [SMILES: O=S(=O)(Nc1nc(Oc2ccccc2)c2ccccc2n1)c1ccccc1] |
| 32 | 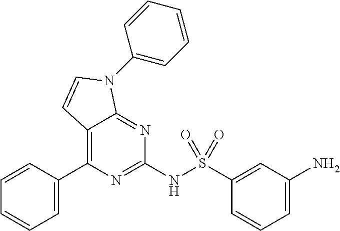 [SMILES: Nc1cccc(S(=O)(=O)Nc2nc(-c3ccccc3)c3ccn(-c4ccccc4)c3n2)c1] |
| 33 | 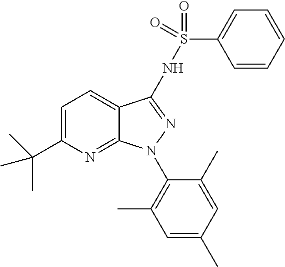 [SMILES: Cc1cc(C)c(-n2nc(NS(=O)(=O)c3ccccc3)c3ccc(C(C)(C)C)nc32)c(C)c1] |
| 34 | 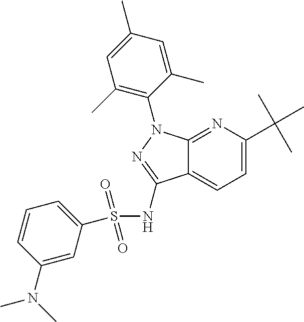 [SMILES: Cc1cc(C)c(-n2nc(NS(=O)(=O)c3cccc(N(C)C)c3)c3ccc(C(C)(C)C)nc32)c(C)c1] |
| 35 | 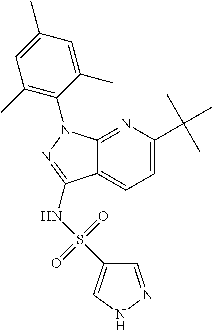 [SMILES: Cc1cc(C)c(-n2nc(NS(=O)(=O)c3cn[nH]c3)c3ccc(C(C)(C)C)nc32)c(C)c1] |
| 36 | 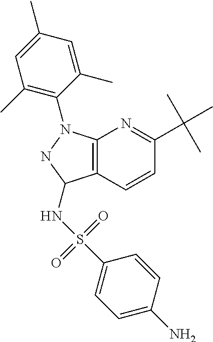 [SMILES: Cc1cc(C)c(N2NC(NS(=O)(=O)c3ccc(N)cc3)c3ccc(C(C)(C)C)nc32)c(C)c1] |
| 37 | 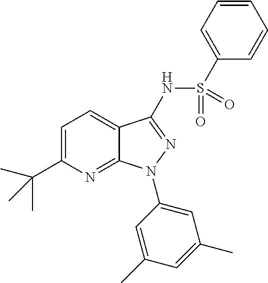 [SMILES: Cc1cc(C)cc(-n2nc(NS(=O)(=O)c3ccccc3)c3ccc(C(C)(C)C)nc32)c1] |
| 38 | 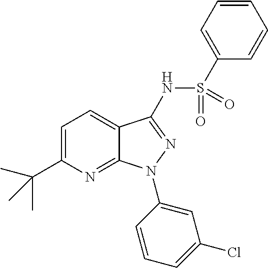 [SMILES: CC(C)(C)c1ccc2c(NS(=O)(=O)c3ccccc3)nn(-c3cccc(Cl)c3)c2n1] |
| 39 | 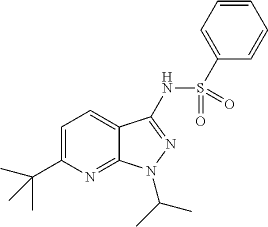 [SMILES: CC(C)n1nc(NS(=O)(=O)c2ccccc2)c2ccc(C(C)(C)C)nc21] |
| 40 | 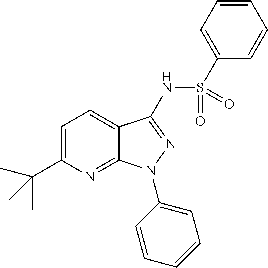 [SMILES: CC(C)(C)c1ccc2c(NS(=O)(=O)c3ccccc3)nn(-c3ccccc3)c2n1] |
| 41 | 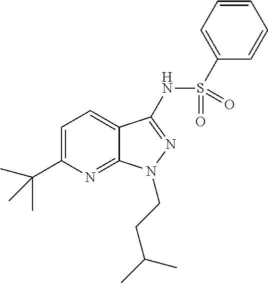 [SMILES: CC(C)CCn1nc(NS(=O)(=O)c2ccccc2)c2ccc(C(C)(C)C)nc21] |
| 42 | 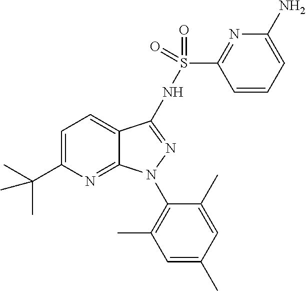 [SMILES: Cc1cc(C)c(-n2nc(NS(=O)(=O)c3cccc(N)n3)c3ccc(C(C)(C)C)nc32)c(C)c1] |
| 43 | 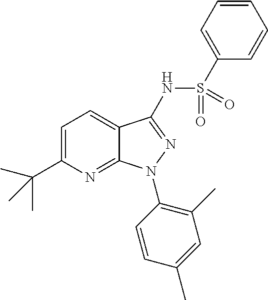 [SMILES: Cc1ccc(-n2nc(NS(=O)(=O)c3ccccc3)c3ccc(C(C)(C)C)nc32)c(C)c1] |
| 44 | 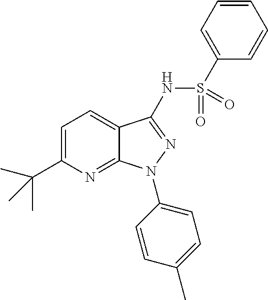 [SMILES: Cc1ccc(-n2nc(NS(=O)(=O)c3ccccc3)c3ccc(C(C)(C)C)nc32)cc1] |
| 45 | 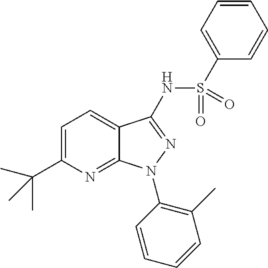 [SMILES: Cc1ccccc1-n1nc(NS(=O)(=O)c2ccccc2)c2ccc(C(C)(C)C)nc21] |
| 46 | 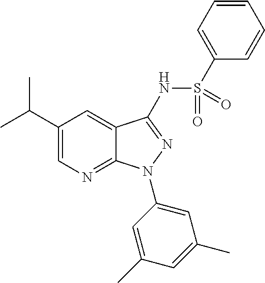 [SMILES: Cc1cc(C)cc(-n2nc(NS(=O)(=O)c3ccccc3)c3cc(C(C)C)cnc32)c1] |
| 47 | 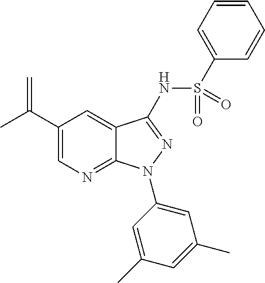 [SMILES: C=C(C)c1cnc2c(c1)c(NS(=O)(=O)c1ccccc1)nn2-c1cc(C)cc(C)c1] |
| 48 | 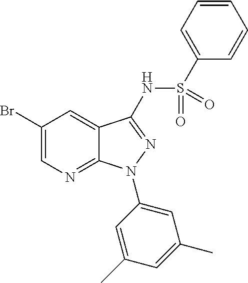 [SMILES: Cc1cc(C)cc(-n2nc(NS(=O)(=O)c3ccccc3)c3cc(Br)cnc32)c1] |
| 49 | 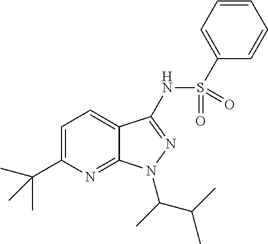 [SMILES: CC(C)C(C)n1nc(NS(=O)(=O)c2ccccc2)c2ccc(C(C)(C)C)nc21] |
| 50 | 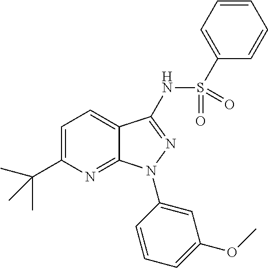 [SMILES: COc1cccc(-n2nc(NS(=O)(=O)c3ccccc3)c3ccc(C(C)(C)C)nc32)c1] |
| 51 | 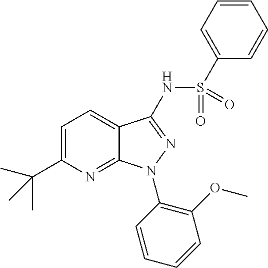 [SMILES: COc1ccccc1-n1nc(NS(=O)(=O)c2ccccc2)c2ccc(C(C)(C)C)nc21] |
| 52 | 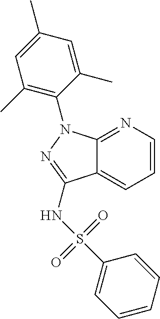 [SMILES: Cc1cc(C)c(-n2nc(NS(=O)(=O)c3ccccc3)c3cccnc32)c(C)c1] |
| 53 | 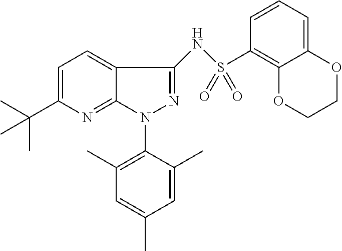 [SMILES: Cc1cc(C)c(-n2nc(NS(=O)(=O)c3cccc4c3OCCO4)c3ccc(C(C)(C)C)nc32)c(C)c1] |
| 54 | 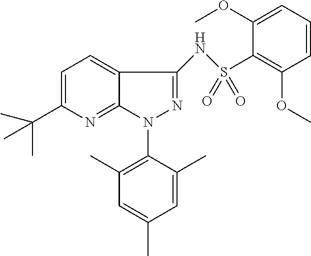 [SMILES: COc1cccc(OC)c1S(=O)(=O)Nc1nn(-c2c(C)cc(C)cc2C)c2nc(C(C)(C)C)ccc12] |
| 55 | 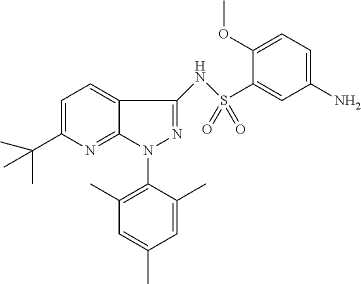 [SMILES: COc1ccc(N)cc1S(=O)(=O)Nc1nn(-c2c(C)cc(C)cc2C)c2nc(C(C)(C)C)ccc12] |
| 56 | 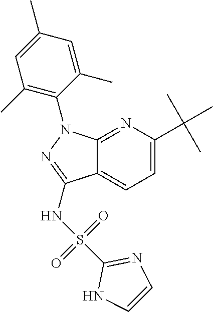 [SMILES: Cc1cc(C)c(-n2nc(NS(=O)(=O)c3ncc[nH]3)c3ccc(C(C)(C)C)nc32)c(C)c1] |
| 57 | 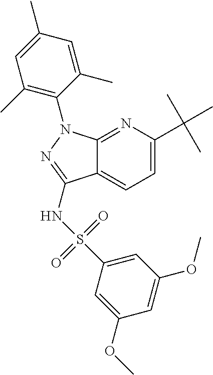 [SMILES: COc1cc(OC)cc(S(=O)(=O)Nc2nn(-c3c(C)cc(C)cc3C)c3nc(C(C)(C)C)ccc23)c1] |
| 58 | 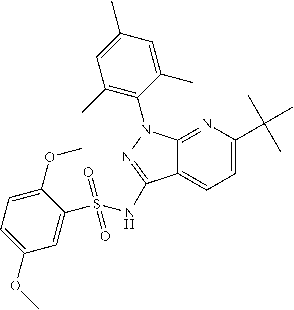 [SMILES: COc1ccc(OC)c(S(=O)(=O)Nc2nn(-c3c(C)cc(C)cc3C)c3nc(C(C)(C)C)ccc23)c1] |
| 59 | 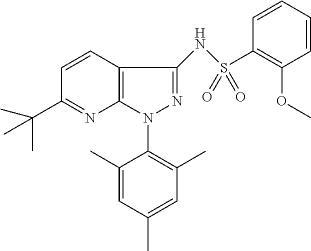 [SMILES: COc1ccccc1S(=O)(=O)Nc1nn(-c2c(C)cc(C)cc2C)c2nc(C(C)(C)C)ccc12] |
| 60 | 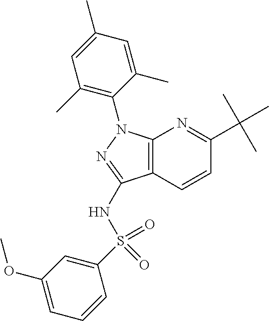 [SMILES: COc1cccc(S(=O)(=O)Nc2nn(-c3c(C)cc(C)cc3C)c3nc(C(C)(C)C)ccc23)c1] |
| 61 | 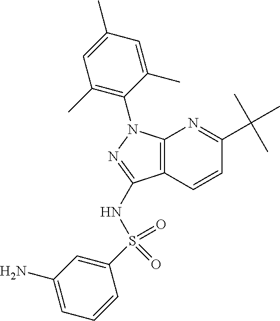 [SMILES: Cc1cc(C)c(-n2nc(NS(=O)(=O)c3cccc(N)c3)c3ccc(C(C)(C)C)nc32)c(C)c1] |
| 62 | 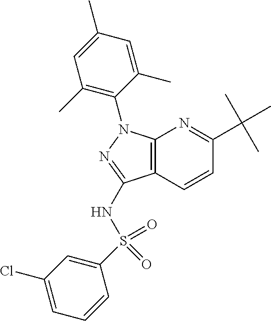 [SMILES: Cc1cc(C)c(-n2nc(NS(=O)(=O)c3cccc(Cl)c3)c3ccc(C(C)(C)C)nc32)c(C)c1] |
| 63 | 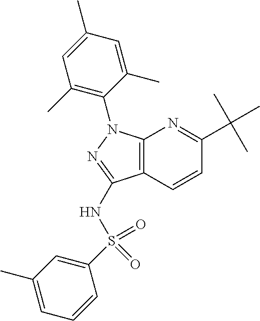 [SMILES: Cc1cccc(S(=O)(=O)Nc2nn(-c3c(C)cc(C)cc3C)c3nc(C(C)(C)C)ccc23)c1] |
| 64 | 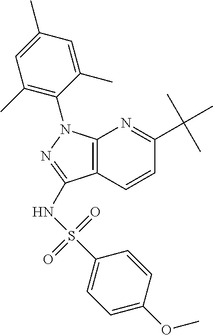 [SMILES: COc1ccc(S(=O)(=O)Nc2nn(-c3c(C)cc(C)cc3C)c3nc(C(C)(C)C)ccc23)cc1] |
| 65 | 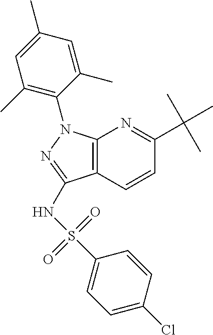 [SMILES: Cc1cc(C)c(-n2nc(NS(=O)(=O)c3ccc(Cl)cc3)c3ccc(C(C)(C)C)nc32)c(C)c1] |
| 66 | 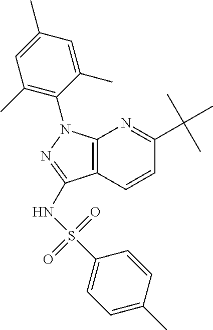 [SMILES: Cc1ccc(S(=O)(=O)Nc2nn(-c3c(C)cc(C)cc3C)c3nc(C(C)(C)C)ccc23)cc1] |
| 67 | 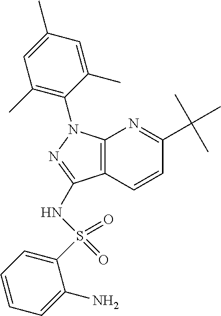 [SMILES: Cc1cc(C)c(-n2nc(NS(=O)(=O)c3ccccc3N)c3ccc(C(C)(C)C)nc32)c(C)c1] |
| 68 | 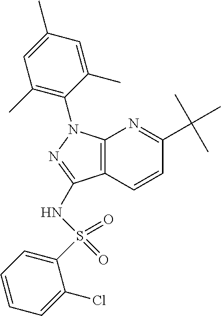 [SMILES: Cc1cc(C)c(-n2nc(NS(=O)(=O)c3ccccc3Cl)c3ccc(C(C)(C)C)nc32)c(C)c1] |
| 69 | 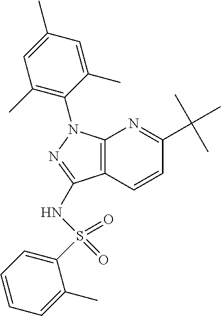 [SMILES: Cc1cc(C)c(-n2nc(NS(=O)(=O)c3ccccc3C)c3ccc(C(C)(C)C)nc32)c(C)c1] |
| 70 | 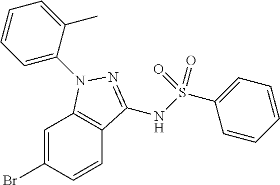 [SMILES: Cc1ccccc1-n1nc(NS(=O)(=O)c2ccccc2)c2ccc(Br)cc21] |
| 71 | 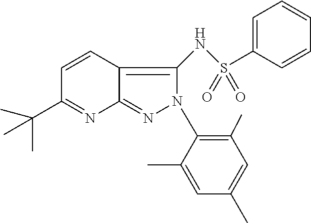 [SMILES: Cc1cc(C)c(-n2nc3nc(C(C)(C)C)ccc3c2NS(=O)(=O)c2ccccc2)c(C)c1] |
| 72 | 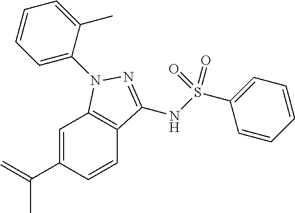 [SMILES: C=C(C)c1ccc2c(NS(=O)(=O)c3ccccc3)nn(-c3ccccc3C)c2c1] |
| 73 | 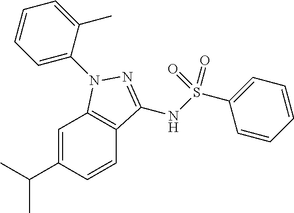 [SMILES: Cc1ccccc1-n1nc(NS(=O)(=O)c2ccccc2)c2ccc(C(C)C)cc21] |
| 74 | 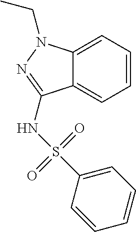 [SMILES: CCn1nc(NS(=O)(=O)c2ccccc2)c2ccccc21] |
| 75 | 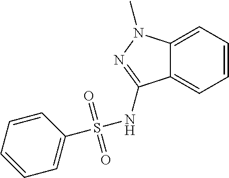 [SMILES: Cn1nc(NS(=O)(=O)c2ccccc2)c2ccccc21] |
| 76 | 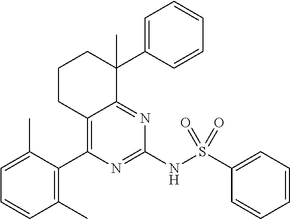 [SMILES: Cc1cccc(C)c1-c1nc(NS(=O)(=O)c2ccccc2)nc2c1CCCC2(C)c1ccccc1] |
| 77 | 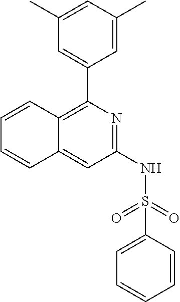 [SMILES: Cc1cc(C)cc(-c2nc(NS(=O)(=O)c3ccccc3)cc3ccccc23)c1] |
| 78 | 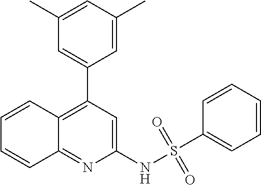 [SMILES: Cc1cc(C)cc(-c2cc(NS(=O)(=O)c3ccccc3)nc3ccccc23)c1] |
| 79 | 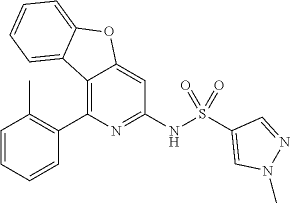 [SMILES: Cc1ccccc1-c1nc(NS(=O)(=O)c2cnn(C)c2)cc2oc3ccccc3c12] |
| 80 | 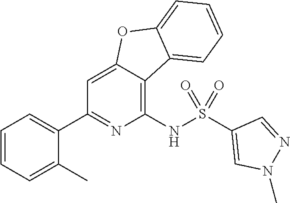 [SMILES: Cc1ccccc1-c1cc2oc3ccccc3c2c(NS(=O)(=O)c2cnn(C)c2)n1] |
| 81 | 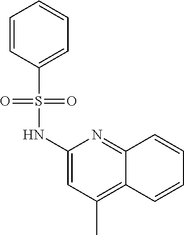 [SMILES: Cc1cc(NS(=O)(=O)c2ccccc2)nc2ccccc12] |
| 82 | 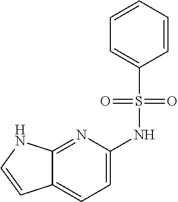 [SMILES: O=S(=O)(Nc1ccc2cc[nH]c2n1)c1ccccc1] |
| 83 | 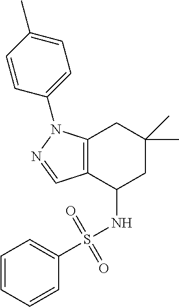 [SMILES: Cc1ccc(-n2ncc3c2CC(C)(C)CC3NS(=O)(=O)c2ccccc2)cc1] |
| 84 | 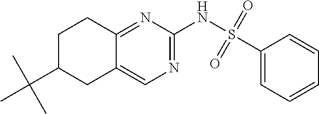 [SMILES: CC(C)(C)C1CCc2nc(NS(=O)(=O)c3ccccc3)ncc2C1] |
| 85 | 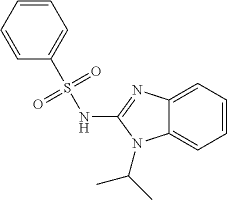 [SMILES: CC(C)n1c(NS(=O)(=O)c2ccccc2)nc2ccccc21] |
| 86 | 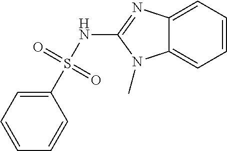 [SMILES: Cn1c(NS(=O)(=O)c2ccccc2)nc2ccccc21] |
| 87 | 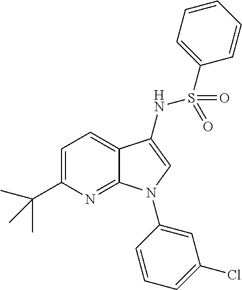 [SMILES: CC(C)(C)c1ccc2c(NS(=O)(=O)c3ccccc3)cn(-c3cccc(Cl)c3)c2n1] |
| 88 | 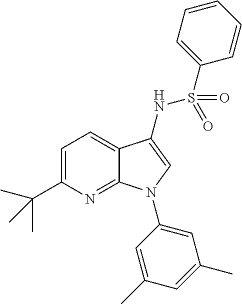 [SMILES: Cc1cc(C)cc(-n2cc(NS(=O)(=O)c3ccccc3)c3ccc(C(C)(C)C)nc32)c1] |
| 89 | 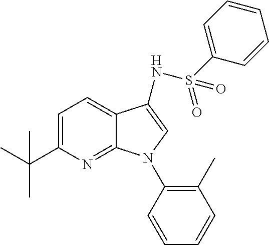 [SMILES: Cc1ccccc1-n1cc(NS(=O)(=O)c2ccccc2)c2ccc(C(C)(C)C)nc21] |
| 90 | 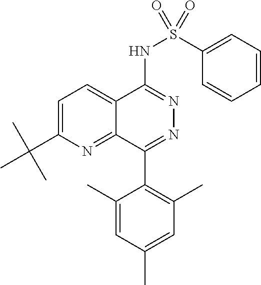 [SMILES: Cc1cc(C)c(-c2nnc(NS(=O)(=O)c3ccccc3)c3ccc(C(C)(C)C)nc23)c(C)c1] |
| 91 | 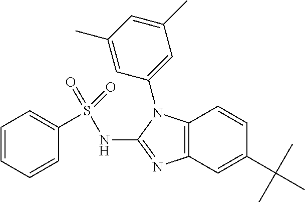 [SMILES: Cc1cc(C)cc(-n2c(NS(=O)(=O)c3ccccc3)nc3cc(C(C)(C)C)ccc32)c1] |
| 92 | 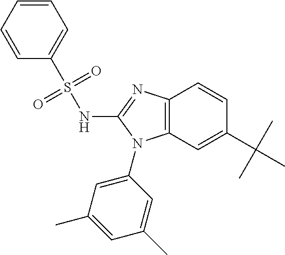 [SMILES: Cc1cc(C)cc(-n2c(NS(=O)(=O)c3ccccc3)nc3ccc(C(C)(C)C)cc32)c1] |
| 93 | 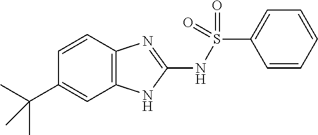 [SMILES: CC(C)(C)c1ccc2nc(NS(=O)(=O)c3ccccc3)[nH]c2c1] |
| 94 | 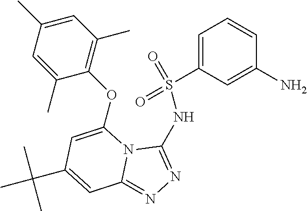 [SMILES: Cc1cc(C)c(Oc2cc(C(C)(C)C)cc3nnc(NS(=O)(=O)c4cccc(N)c4)n23)c(C)c1] |
| 95 | 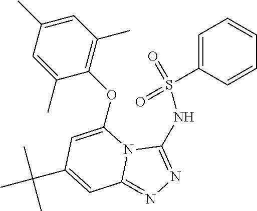 [SMILES: Cc1cc(C)c(Oc2cc(C(C)(C)C)cc3nnc(NS(=O)(=O)c4ccccc4)n23)c(C)c1] |

or a tautomer, deuterated derivative, or pharmaceutically acceptable salt thereof.

24. A pharmaceutical composition comprising a compound, tautomer, deuterated derivative, or pharmaceutically acceptable salt of any one of claims 1-23 and a pharmaceutically acceptable carrier.

25. The pharmaceutical composition of claim 24, further comprising one or more additional therapeutic agent(s).

26. The pharmaceutical composition of claim 25, wherein the one or more additional therapeutic agent(s) comprise(s) a compound selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and pharmaceutically acceptable salts of any of the foregoing.

27. A method of treating cystic fibrosis comprising administering to a patient in need thereof a compound, tautomer, deuterated derivative, or pharmaceutically acceptable salt of any one of claims 1-23 or a pharmaceutical composition according to any one of claims 24-26.

28. The method of claim 27, further comprising administering to the patient one or more additional therapeutic agent(s) prior to, concurrent with, or subsequent to the compound or the pharmaceutical composition.

29. The method of claim 28, wherein the one or more additional therapeutic agent(s) comprise(s) a compound selected from tezacaftor, ivacaftor, deutivacaftor, lumacaftor, (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol, deuterated derivatives of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.

30. The compound, tautomer, deuterated derivative, or pharmaceutically acceptable salt of any one of claims 1-23 or the pharmaceutical composition according to any one of claims 24-26 for use in the treatment of cystic fibrosis.

31. The compound, tautomer, deuterated derivative, or pharmaceutically acceptable salt of any one of claims 1-23 or the pharmaceutical composition according to any one of claims 24-26 for use in the manufacture of a medicament for the treatment of cystic fibrosis.

32. A compound selected from Compounds 1-95, tautomers thereof, deuterated derivatives of those compounds and tautomers, and pharmaceutically acceptable salts of any of the foregoing.

33. A deuterated derivative of a compound selected from Compounds 1-95.

34. A pharmaceutically acceptable salt of a compound selected from Compounds 1-95.

35. A compound selected from Compounds 1-95.

36. A pharmaceutical composition comprising a compound selected from Compounds 1-95, tautomers thereof, deuterated derivatives of those compounds and tautomers, and pharmaceutically acceptable salts of any of the foregoing and a pharmaceutically acceptable carrier.

37. A pharmaceutical composition comprising a deuterated derivative of a compound selected from Compounds 1-95 and a pharmaceutically acceptable carrier.

38. A pharmaceutical composition comprising a pharmaceutically acceptable salt of a compound selected from Compounds 1-95 and a pharmaceutically acceptable carrier.

39. A pharmaceutical composition comprising a compound selected from Compounds 1-95 and a pharmaceutically acceptable carrier.

40. A pharmaceutical composition comprising (a) a compound selected from Compounds 1-95, tautomers thereof, deuterated derivatives of those compounds and tautomers, and pharmaceutically acceptable salts of any of the foregoing; (b) a CFTR potentiator; and (c) a pharmaceutically acceptable carrier.

41. A pharmaceutical composition composition comprising (a) a deuterated derivative of a compound selected from Compounds 1-95; (b) a CFTR potentiator; and (c) a pharmaceutically acceptable carrier.

42. A pharmaceutical comprising (a) a pharmaceutically acceptable salt of a compound selected from Compounds 1-95; (b) a CFTR potentiator; and (c) a pharmaceutically acceptable carrier.

43. A pharmaceutical composition comprising (a) a compound selected from Compounds 1-95; (b) a CFTR potentiator; and (c) a pharmaceutically acceptable carrier.

44. A pharmaceutical composition comprising (a) a compound selected from Compounds 1-95, tautomers thereof, deuterated derivatives of those compounds and tautomers, and pharmaceutically acceptable salts of any of the foregoing; (b) an additional CFTR corrector; and (c) a pharmaceutically acceptable carrier.

45. A pharmaceutical composition comprising (a) a deuterated derivative of a compound selected from Compounds 1-95; (b) an additional CFTR corrector; and (c) a pharmaceutically acceptable carrier.

46. A pharmaceutical composition comprising (a) a pharmaceutically acceptable salt of a compound selected from Compounds 1-95; (b) an additional CFTR corrector; and (c) a pharmaceutically acceptable carrier.

47. A pharmaceutical composition comprising (a) a compound selected from Compounds 1-95; (b) an additional CFTR corrector; and (c) a pharmaceutically acceptable carrier.

48. A pharmaceutical composition comprising (a) a compound selected from Compounds 1-95, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.

49. A pharmaceutical composition comprising (a) a deuterated derivative of a compound selected from Compounds 1-95; (b) an additional CFTR corrector; (c) a CFTR potentiator; and (d) a pharmaceutically acceptable carrier.

50. A pharmaceutical composition comprising (a) a pharmaceutically acceptable salt of a compound selected from Compounds 1-95; (b) an additional CFTR corrector; (c) a CFTR potentiator; and (d) a pharmaceutically acceptable carrier.

51. A pharmaceutical composition comprising (a) a compound selected from Compounds 1-95; (b) an additional CFTR corrector; (c) a CFTR potentiator; and (d) a pharmaceutically acceptable carrier.

52. A compound selected from Compounds 1-95, tautomers thereof, deuterated derivatives of those compounds and tautomers, and pharmaceutically acceptable salts of any of the foregoing for use in a method of treating cystic fibrosis.

53. A deuterated derivative of a compound selected from Compounds 1-95 for use in a method of treating cystic fibrosis.

54. A pharmaceutically acceptable salt of a compound selected from Compounds 1-95 for use in a method of treating cystic fibrosis.

55. A compound selected from Compounds 1-95 for use in a method of treating cystic fibrosis.

56. A pharmaceutical composition comprising a compound selected from Compounds 1-95, tautomers thereof, deuterated derivatives of those compounds and tautomers, and pharmaceutically acceptable salts of any of the foregoing and a pharmaceutically acceptable carrier for use in a method of treating cystic fibrosis.

57. A pharmaceutical composition comprising a deuterated derivative of a compound selected from Compounds 1-95 and a pharmaceutically acceptable carrier for use in a method of treating cystic fibrosis.

58. A pharmaceutical composition comprising a pharmaceutically acceptable salt of a compound selected from Compounds 1-95 and a pharmaceutically acceptable carrier for use in a method of treating cystic fibrosis.

59. A pharmaceutical composition comprising a compound selected from Compounds 1-95 and a pharmaceutically acceptable carrier for use in a method of treating cystic fibrosis.

60. A pharmaceutical composition comprising (a) a compound selected from Compounds 1-95, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.

61. A pharmaceutical comprising (a) a deuterated derivative of a compound selected from Compounds 1-95; (b) a CFTR potentiator; and (c) a pharmaceutically acceptable carrier for use in a method of treating cystic fibrosis.

62. A pharmaceutical composition comprising (a) a pharmaceutically acceptable salt of a compound selected from Compounds 1-95; (b) a CFTR potentiator; and (c) a pharmaceutically acceptable carrier for use in a method of treating cystic fibrosis.

63. A pharmaceutical composition comprising (a) a compound selected from Compounds 1-95; (b) a CFTR potentiator; and (c) a pharmaceutically acceptable carrier.

64. A pharmaceutical composition comprising (a) a compound selected from Compounds 1-95, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.

65. A pharmaceutical composition comprising (a) a deuterated derivative of a compound selected from Compounds 1-95; (b) an additional CFTR corrector; and (c) a pharmaceutically acceptable carrier for use in a method of treating cystic fibrosis.

66. A pharmaceutical composition comprising (a) a pharmaceutically acceptable salt of a compound selected from Compounds 1-95; (b) an additional CFTR corrector; and (c) a pharmaceutically acceptable carrier for use in a method of treating cystic fibrosis.

67. A pharmaceutical composition comprising (a) a compound selected from Compounds 1-95; (b) an additional CFTR corrector; and (c) a pharmaceutically acceptable carrier for use in a method of treating cystic fibrosis.

68. A pharmaceutical composition comprising (a) a compound selected from Compounds 1-95, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.

69. A pharmaceutical composition comprising (a) a deuterated derivative of a compound selected from Compounds 1-95; (b) an additional CFTR corrector; (c) a CFTR potentiator; and (d) a pharmaceutically acceptable carrier for use in a method of treating cystic fibrosis.

70. A pharmaceutical composition comprising (a) a pharmaceutically acceptable salt of a compound selected from Compounds 1-95; (b) an additional CFTR corrector; (c) a CFTR potentiator; and (d) a pharmaceutically acceptable carrier for use in a method of treating cystic fibrosis.

71. A pharmaceutical composition comprising (a) a compound selected from Compounds 1-95; (b) an additional CFTR corrector; (c) a CFTR potentiator; and (d) a pharmaceutically acceptable carrier for use in a method of treating cystic fibrosis.
